(19) United States
(12) Patent Application Publication
(10) Pub. No.: US 2018/0221306 A1
(43) Pub. Date: Aug. 9, 2018

(54) PHARMACEUTICAL COMPOSITIONS OF 5-HYDROXY-2-METHYLNAPHTHALENE-1,4-DIONE

(71) Applicant: PellFiCure Pharmaceuticals, Inc., La Jolla, CA (US)

(72) Inventors: Per Borgström, La Jolla, CA (US); Adrian Chrastina, Chula Vista, CA (US); Veronique Baron, Carlsbad, CA (US)

(21) Appl. No.: 15/922,405

(22) Filed: Mar. 15, 2018

Related U.S. Application Data
(63) Continuation of application No. PCT/US2017/057710, filed on Oct. 20, 2017.
(60) Provisional application No. 62/412,131, filed on Oct. 24, 2016.

(51) Int. Cl. A61K 31/122; A61K 47/14; A61K 9/00; A61K 45/06

(52) U.S. Cl. CPC A61K 31/122; A61K 45/06; A61K 9/0053; A61K 47/14

(57) ABSTRACT

Disclosed herein are pharmaceutical compositions of 5-hydroxy-2-methylnaphthalene-1,4-dione. Also disclosed are methods of treating diseases and/or conditions associated with a cancer, such as prostate cancer with such pharmaceutical compositions of 5-hydroxy-2-methylnaphthalene-1,4-dione. The disclosed pharmaceutical compositions may provide improved dosage for oral administration to patients in the clinic. The disclosed pharmaceutical compositions may provide improved stability and/or shelf life.

### CROSS-REFERENCE TO RELATED APPLICATIONS

This application claims the benefit of priority to U.S. Provisional Application No. 62/412,131 filed Oct. 24, 2016, the disclosure of which is hereby expressly incorporated by reference in its entirety.

### FIELD

The present disclosure relates in general to 5-hydroxy-2-methylnaphthalene-1,4-dione. Specifically, the present disclosure relates to pharmaceutical compositions containing 5-hydroxy-2-methylnaphthalene-1,4-dione. Further provided are methods of using such pharmaceutical compositions for treating and/or ameliorating diseases and/or conditions associated with a cancer, such as prostate cancer.

### BACKGROUND

Prostate cancer is the second leading cause of cancer-related death in American men. Current therapeutic treatments for prostate cancer may prolong life in patients, but the survival benefit is limited. Unfortunately most patients treated with conventional hormone therapy eventually relapse, and most patients treated with hormone therapy eventually develop castration-resistant prostate cancer (CRPC). Current treatments for CRPC are palliative only. New treatments are needed to inhibit, delay, and prevent the onset of CRPC.

5-Hydroxy-2-methylnaphthalene-1,4-dione can be administered to animals by intra-peritoneal injection or oral gavage after dissolution in various organic chemistry solvents or in polyethylene glycol (PEG). These methods of administration are undesirable for human usage in a clinical setting. There is a need for new oral formulations of 5-hydroxy-2-methylnaphthalene-1,4-dione that are acceptable for uses by human patients in a clinical setting.

### SUMMARY

It is therefore an aspect of this disclosure to provide improved pharmaceutical compositions of 5-hydroxy-2-methylnaphthalene-1,4-dione. It is a related aspect to provide pharmaceutical compositions of 5-hydroxy-2-methylnaphthalene-1,4-dione capable of eliciting and sustaining desirable responses in a patient in need thereof. It is another aspect of this disclosure to provide methods for treating and/or ameliorating diseases and/or conditions associated with a cancer, such as prostate cancer, using such compositions.

Some embodiments disclosed herein relate to methods for increasing survival of a subject or treating a subject suffering from prostate cancer that can include identifying a subject at risk for reduced prostate cancer survival or a subject having prostate cancer, and administering to the subject a pharmaceutical composition comprising Compound I and at least one pharmaceutically acceptable carrier, where Compound I is 5-hydroxy-2-methylnaphthalene-1,4-dione. In some embodiments, the at least one pharmaceutically acceptable carrier can include a mixture of triacylglycerols. In some embodiments, the mixture of triacylglycerols can include glyceryl esters of one or more fatty acids selected from the group consisting of caproic acid, caprylic acid, capric acid, lauric acid, myristic acid, palmitic acid, stearic acid, oleic acid, linolenic acid, linoleic acid, arachidic acid, ricinoleic acid, dihydroxystearic acid, behenic acid, ligoceric acid, erucic acid, and gondoic acid. In some embodiments, the at least one pharmaceutically acceptable carrier can be a liquid at 25° C. In some embodiments, Compound I can be dissolved in the at least one pharmaceutically acceptable carrier at a concentration between 0.05 to 100 mg of Compound I per mL of pharmaceutically acceptable carrier. In some embodiments, the administration of the pharmaceutical composition may result in survival of a subject for more than 1 month. In some embodiments, when Compound I is dissolved in the at least one pharmaceutically acceptable carrier at 25° C., the resulting mixture is a liquid having a viscosity between 20-250 milliPascal seconds (mPa·s). In some embodiments, when Compound I is dissolved in the at least one pharmaceutically acceptable carrier at 37° C., the resulting mixture is a liquid having a viscosity between 20-250 milliPascal seconds (mPa·s).

Some embodiments disclosed herein that relate to methods for increasing survival in a subject suffering from prostate cancer include a subject at risk for reduced prostate cancer survival that has a ruptured or broken prostate capsule, or metastatic prostate cancer. Some embodiments disclosed herein that relate to methods for increasing survival in a subject suffering from prostate cancer include a subject at risk for reduced prostate cancer survival that has previously undergone surgical castration. Some embodiments disclosed herein that relate to methods for increasing survival in a subject suffering from prostate cancer include administering to the subject androgen deprivation therapy that reduces the amount of androgen in the subject. In some embodiments, the androgen deprivation therapy can be selected from the group consisting of abiraterone, dutasteride, degarelix, and leuprolide.

Some embodiments disclosed herein that relate to methods for increasing survival in a subject suffering from prostate cancer can include identifying a subject at risk for reduced prostate cancer survival, and administering to the subject a pharmaceutical composition comprising Compound I and at least one pharmaceutically acceptable carrier, where the at least one pharmaceutically acceptable carrier can include a mixture of triacylglycerols. In some embodiments, the mixture of triacylglycerols can include glyceryl esters of oleic acid, linoleic acid, palmitic acid, stearic acid, and arachidic acid. In some embodiments, the mixture of triacylglycerols can include glyceryl esters of ricinoleic acid, oleic acid, linoleic acid, palmitic acid, stearic acid, and dihydroxystearic acid. In some embodiments, the mixture of triacylglycerols can include glyceryl esters of lauric acid, myristic acid, palmitic acid, oleic acid, caprylic acid, stearic acid, capric acid, caproic acid, linoleic acid, and arachidic acid. In some embodiments, the mixture of triacylglycerols can include glyceryl esters of linoleic acid, oleic acid, palmitic acid, capric acid, caprylic acid, stearic acid, and myristic acid. In some embodiments, the mixture of triacylglycerols can include glyceryl esters of linoleic acid, oleic acid, palmitic acid, stearic acid, and myristic acid. In some embodiments, the mixture of triacylglycerols can include glyceryl esters of linoleic acid, oleic acid, palmitic acid, stearic acid, alpha-linolenic acid, and palmitoleic acid. In some embodiments, the mixture of triacylglycerols can include glyceryl esters of oleic acid, palmitic acid, linoleic acid, stearic acid, myristic acid, and arachidic acid. In some embodiments, the mixture of triacylglycerols can include glyceryl esters of oleic acid, linoleic acid, palmitic acid, stearic acid, behenic acid, arachidic acid, and lignoceric acid. In some embodiments, the mixture of triacylglycerols can include glyceryl esters of erucic acid, oleic acid, gondonic acid, linoleic acid, alpha-linolenic acid, palmitic acid, and stearic acid. In some embodiments, the mixture of triacylglycerols can include glyceryl esters of oleic acid, linoleic acid, alpha-linolenic acid, palmitic acid, gondonic acid, and stearic acid. In some embodiments, the mixture of triacylglycerols can include glyceryl esters of linoleic acid, oleic acid, palmitic acid, and stearic acid. In some embodiments, the mixture of triacylglycerols can include glyceryl esters of linoleic acid, oleic acid, palmitic acid, linolenic acid, and stearic acid. In some embodiments, the mixture of triacylglycerols can include glyceryl esters of linoleic acid, oleic acid, palmitic acid, stearic acid, arachidic acid, and behenic acid.

Some embodiments disclosed herein that relate to methods for increasing survival in a subject suffering from prostate cancer can include identifying a subject at risk for reduced prostate cancer survival, and administering to the subject a pharmaceutical composition comprising Compound I and at least one pharmaceutically acceptable carrier, where the at least one pharmaceutically acceptable carrier can include a mixture of triacylglycerols. In some embodiments, the mixture of triacylglycerols can include glyceryl esters having a fatty acid content of 40-50% oleic acid, 35-45% linoleic acid, 7-9% palmitic acid, 4-5% stearic acid, and 0.4-1% arachidic acid. In some embodiments, the mixture of triacylglycerols can include glyceryl esters having a fatty acid content of 85-95% ricinoleic acid, 2-8% oleic acid, 1-6% linoleic acid, 0.5-3% palmitic acid, 0.5-1% stearic acid, and 0.3-0.7% dihydroxystearic acid. In some embodiments, the mixture of triacylglycerols can include glyceryl esters having a fatty acid content of 44-52% lauric acid, 13-19% myristic acid, 8-11% palmitic acid, 6-10% capric acid, 5-8% oleic acid, 5-9% caprylic acid, 1-3% stearic acid, 0-1% linoleic acid, 0-0.8% caproic acid, and 0-0.5% arachidic acid. In some embodiments, the mixture of triacylglycerols can include glyceryl esters having a fatty acid content of 34-62% linoleic acid, 19-49% oleic acid, 8-12% palmitic acid, 7% capric acid, 4% caprylic acid, 2-5% stearic acid, and 0.2-1% myristic acid. In some embodiments, the mixture of triacylglycerols can include glyceryl esters having a fatty acid content of 40-63% linoleic acid, 13-44% oleic acid, 17-29% palmitic acid, 1-4% stearic acid, 0.5-2% myristic acid, and 0.1-2% alpha-linolenic acid. In some embodiments, the mixture of triacylglycerols can include glyceryl esters having a fatty acid content of 60-75% linoleic acid, 12-25% oleic acid, 6-9% palmitic acid, 3-6% stearic acid, 0-1.5% alpha-linolenic acid, and 0-1% palmitoleic acid. In some embodiments, the mixture of triacylglycerols can include glyceryl esters having a fatty acid content of 65-80% oleic acid, 7-16% palmitic acid, 4-10% linoleic acid, 1-3% stearic acid, 0.1-1% myristic acid, and 0.1-0.3% arachidic acid. In some embodiments, the mixture of triacylglycerols can include glyceryl esters having a fatty acid content of 38-52% oleic acid, 32-45% palmitic acid, 5-11% linoleic acid, 2-7% stearic acid, and 0.5-2% myristic acid. In some embodiments, the mixture of triacylglycerols can include glyceryl esters having a fatty acid content of 47-56% oleic acid, 26-33% linoleic acid, and 8-10% palmitic acid. In some embodiments, the mixture of triacylglycerols can include glyceryl esters having a fatty acid content of 41% erucic acid, 17% oleic acid, 15% gondonic acid, 13% linoleic acid, 9% alpha-linolenic acid, 4% palmitic acid, and 1.5% stearic acid. In some embodiments, the mixture of triacylglycerols can include glyceryl esters having a fatty acid content of 61-63% oleic acid, 20-21% linoleic acid, 9-11% alpha-linolenic acid, 4% palmitic acid, 2% gondonic acid, 2% stearic acid, and less than 2% erucic acid. In some embodiments, the mixture of triacylglycerols can include glyceryl esters having a fatty acid content of 73-79% linoleic acid, 13-21% oleic acid, 3-6% palmitic acid, and 1-4% stearic acid. In some embodiments, the mixture of triacylglycerols can include glyceryl esters having a fatty acid content of 43-56% linoleic acid, 22-34% oleic acid, 7-11% palmitic acid, 5-11% linolenic acid, and 2-6% stearic acid. In some embodiments, the mixture of triacylglycerols can include glyceryl esters having a fatty acid content of 44-75% linoleic acid, 14-35% oleic acid, 3-6% palmitic acid, 1-3% stearic acid, 0.6-4% arachidic acid, and 1% behenic acid.

Some embodiments disclosed herein that relate to methods for increasing survival in a subject suffering from prostate cancer can include identifying a subject at risk for reduced prostate cancer survival, and administering to the subject a pharmaceutical composition comprising Compound I and at least one pharmaceutically acceptable carrier. In some embodiments, the amount of Compound I in the pharmaceutical composition can be selected from the group consisting of 25 mg, 50 mg, 75 mg, 100 mg, 125 mg, 150 mg, 200 mg, and 250 mg. In some embodiments, the subject can be administered a dosage amount of 1 mg of Compound I per kg of subject, and the pharmaceutical composition can be formulated for oral administration to the patient.

Some embodiments disclosed herein relate to methods for inhibiting, delaying, or preventing rupture of a subject's prostatic capsule in a subject suffering from prostate cancer that can include administering to the subject a pharmaceutical composition comprising Compound I and at least one pharmaceutically acceptable carrier, where Compound I is 5-hydroxy-2-methylnaphthalene-1,4-dione. In some embodiments, the at least one pharmaceutically acceptable carrier can include a mixture of triacylglycerols where the triacylglycerols can include glyceryl esters of one or more fatty acids selected from the group consisting of caproic acid, caprylic acid, capric acid, lauric acid, myristic acid, palmitic acid, stearic acid, oleic acid, linolenic acid, linoleic acid, arachidic acid, ricinoleic acid, dihydroxystearic acid, behenic acid, ligoceric acid, erucic acid, and gondoic acid. In some embodiments, the pharmaceutically acceptable carrier can be a liquid at 25° C. In some embodiments, Compound I can be dissolved in the pharmaceutically acceptable carrier at a concentration between 0.05 to 100 mg of Compound I per mL of pharmaceutically acceptable carrier. In some embodiments, when Compound I is dissolved in the at least one pharmaceutically acceptable carrier at 25° C., the resulting mixture is a liquid having a viscosity between 20-250 milliPascal seconds (mPa·s). In some embodiments, when Compound I is dissolved in the at least one pharmaceutically acceptable carrier at 37° C., the resulting mixture is a liquid having a viscosity between 20-250 milliPascal seconds (mPa·s).

Some embodiments disclosed herein that relate to methods for inhibiting, delaying, or preventing rupture of a subject's prostatic capsule in a subject suffering from prostate cancer include a subject at risk for reduced prostate cancer survival that has previously undergone surgical castration. Some embodiments disclosed herein that relate to methods for inhibiting, delaying, or preventing rupture of a subject's prostatic capsule in a subject suffering from prostate cancer include administering to the subject androgen deprivation therapy that reduces the amount of androgen in the subject. In some embodiments, the androgen deprivation therapy can be selected from the group consisting of abiraterone, dutasteride, degarelix, and leuprolide.

Some embodiments disclosed herein that relate to methods for inhibiting, delaying, or preventing rupture of a subject's prostatic capsule in a subject suffering from prostate cancer include administering to the subject a pharmaceutical composition comprising Compound I and at least one pharmaceutically acceptable carrier, where the at least one pharmaceutically acceptable carrier can include a mixture of triacylglycerols. In some embodiments, the mixture of triacylglycerols can include glyceryl esters of oleic acid, linoleic acid, palmitic acid, stearic acid, and arachidic acid. In some embodiments, the mixture of triacylglycerols can include glyceryl esters of ricinoleic acid, oleic acid, linoleic acid, palmitic acid, stearic acid, and dihydroxystearic acid. In some embodiments, the mixture of triacylglycerols can include glyceryl esters of lauric acid, myristic acid, palmitic acid, oleic acid, caprylic acid, stearic acid, capric acid, caproic acid, linoleic acid, and arachidic acid. In some embodiments, the mixture of triacylglycerols can include glyceryl esters of linoleic acid, oleic acid, palmitic acid, capric acid, caprylic acid, stearic acid, and myristic acid. In some embodiments, the mixture of triacylglycerols can include glyceryl esters of linoleic acid, oleic acid, palmitic acid, stearic acid, and myristic acid. In some embodiments, the mixture of triacylglycerols can include glyceryl esters of linoleic acid, oleic acid, palmitic acid, stearic acid, alpha-linolenic acid, and palmitoleic acid. In some embodiments, the mixture of triacylglycerols can include glyceryl esters of oleic acid, palmitic acid, linoleic acid, stearic acid, myristic acid, and arachidic acid. In some embodiments, the mixture of triacylglycerols can include glyceryl esters of oleic acid, linoleic acid, palmitic acid, stearic acid, behenic acid, arachidic acid, and lignoceric acid. In some embodiments, the mixture of triacylglycerols can include glyceryl esters of erucic acid, oleic acid, gondonic acid, linoleic acid, alpha-linolenic acid, palmitic acid, and stearic acid. In some embodiments, the mixture of triacylglycerols can include glyceryl esters of oleic acid, linoleic acid, alpha-linolenic acid, palmitic acid, gondonic acid, and stearic acid. In some embodiments, the mixture of triacylglycerols can include glyceryl esters of linoleic acid, oleic acid, palmitic acid, and stearic acid. In some embodiments, the mixture of triacylglycerols can include glyceryl esters of linoleic acid, oleic acid, palmitic acid, linolenic acid, and stearic acid. In some embodiments, the mixture of triacylglycerols can include glyceryl esters of linoleic acid, oleic acid, palmitic acid, stearic acid, arachidic acid, and behenic acid.

Some embodiments disclosed herein that relate to methods for inhibiting, delaying, or preventing rupture of a subject's prostatic capsule in a subject suffering from prostate cancer include administering to the subject a pharmaceutical composition comprising Compound I and at least one pharmaceutically acceptable carrier, where the at least one pharmaceutically acceptable carrier can include a mixture of triacylglycerols. In some embodiments, the mixture of triacylglycerols can include glyceryl esters having a fatty acid content of 40-50% oleic acid, 35-45% linoleic acid, 7-9% palmitic acid, 4-5% stearic acid, and 0.4-1% arachidic acid. In some embodiments, the mixture of triacylglycerols can include glyceryl esters having a fatty acid content of 85-95% ricinoleic acid, 2-8% oleic acid, 1-6% linoleic acid, 0.5-3% palmitic acid, 0.5-1% stearic acid, and 0.3-0.7% dihydroxystearic acid. In some embodiments, the mixture of triacylglycerols can include glyceryl esters having a fatty acid content of 44-52% lauric acid, 13-19% myristic acid, 8-11% palmitic acid, 6-10% capric acid, 5-8% oleic acid, 5-9% caprylic acid, 1-3% stearic acid, 0-1% linoleic acid, 0-0.8% caproic acid, and 0-0.5% arachidic acid. In some embodiments, the mixture of triacylglycerols can include glyceryl esters having a fatty acid content of 34-62% linoleic acid, 19-49% oleic acid, 8-12% palmitic acid, 7% capric acid, 4% caprylic acid, 2-5% stearic acid, and 0.2-1% myristic acid. In some embodiments, the mixture of triacylglycerols can include glyceryl esters having a fatty acid content of 40-63% linoleic acid, 13-44% oleic acid, 17-29% palmitic acid, 1-4% stearic acid, 0.5-2% myristic acid, and 0.1-2% alpha-linolenic acid. In some embodiments, the mixture of triacylglycerols can include glyceryl esters having a fatty acid content of 60-75% linoleic acid, 12-25% oleic acid, 6-9% palmitic acid, 3-6% stearic acid, 0-1.5% alpha-linolenic acid, and 0-1% palmitoleic acid. In some embodiments, the mixture of triacylglycerols can include glyceryl esters having a fatty acid content of 65-80% oleic acid, 7-16% palmitic acid, 4-10% linoleic acid, 1-3% stearic acid, 0.1-1% myristic acid, and 0.1-0.3% arachidic acid. In some embodiments, the mixture of triacylglycerols can include glyceryl esters having a fatty acid content of 38-52% oleic acid, 32-45% palmitic acid, 5-11% linoleic acid, 2-7% stearic acid, and 0.5-2% myristic acid. In some embodiments, the mixture of triacylglycerols can include glyceryl esters having a fatty acid content of 47-56% oleic acid, 26-33% linoleic acid, and 8-10% palmitic acid. In some embodiments, the mixture of triacylglycerols can include glyceryl esters having a fatty acid content of 41% erucic acid, 17% oleic acid, 15% gondonic acid, 13% linoleic acid, 9% alpha-linolenic acid, 4% palmitic acid, and 1.5% stearic acid. In some embodiments, the mixture of triacylglycerols can include glyceryl esters having a fatty acid content of 61-63% oleic acid, 20-21% linoleic acid, 9-11% alpha-linolenic acid, 4% palmitic acid, 2% gondonic acid, 2% stearic acid, and less than 2% erucic acid. In some embodiments, the mixture of triacylglycerols can include glyceryl esters having a fatty acid content of 73-79% linoleic acid, 13-21% oleic acid, 3-6% palmitic acid, and 1-4% stearic acid. In some embodiments, the mixture of triacylglycerols can include glyceryl esters having a fatty acid content of 43-56% linoleic acid, 22-34% oleic acid, 7-11% palmitic acid, 5-11% linolenic acid, and 2-6% stearic acid. In some embodiments, the mixture of triacylglycerols can include glyceryl esters having a fatty acid content of 44-75% linoleic acid, 14-35% oleic acid, 3-6% palmitic acid, 1-3% stearic acid, 0.6-4% arachidic acid, and 1% behenic acid.

Some embodiments disclosed herein that relate to methods for inhibiting, delaying, or preventing rupture of a subject's prostatic capsule in a subject suffering from prostate cancer include administering to the subject a pharmaceutical composition comprising Compound I and at least one pharmaceutically acceptable carrier. In some embodiments, the amount of Compound I in the pharmaceutical composition can be selected from the group consisting of 25 mg, 50 mg, 75 mg, 100 mg, 125 mg, 150 mg, 200 mg, and 250 mg. In some embodiments, the subject can be administered a dosage amount of 1 mg of Compound I per kg of subject, and the pharmaceutical composition can be formulated for oral administration to the patient.

Some embodiments disclosed herein relate to a pharmaceutical composition that includes Compound I and at least one pharmaceutically acceptable carrier, where Compound I is 5-hydroxy-2-methylnaphthalene-1,4-dione. In some embodiments, the at least one pharmaceutically acceptable carrier can include a mixture of triacylglycerols where the triacylglycerols can include glyceryl esters of one or more fatty acids selected from the group consisting of caproic acid, caprylic acid, capric acid, lauric acid, myristic acid, palmitic acid, stearic acid, oleic acid, linolenic acid, linoleic acid, arachidic acid, ricinoleic acid, dihydroxystearic acid, behenic acid, ligoceric acid, erucic acid, and gondoic acid. In some embodiments, the pharmaceutically acceptable carrier can be a liquid at 25° C. In some embodiments, Compound I can be dissolved in the pharmaceutically acceptable carrier at a concentration between 0.05 to 100 mg of Compound I per mL of pharmaceutically acceptable carrier. In some embodiments, when Compound I is dissolved in the at least one pharmaceutically acceptable carrier at 25° C., the resulting mixture is a liquid having a viscosity between 20-250 milliPascal seconds (mPa·s). In some embodiments, when Compound I is dissolved in the at least one pharmaceutically acceptable carrier at 37° C., the resulting mixture is a liquid having a viscosity between 20-250 milliPascal seconds (mPa·s). In some embodiments, the amount of Compound I in the pharmaceutical composition can be selected from the group consisting of 25 mg, 50 mg, 75 mg, 100 mg, 125 mg, 150 mg, 200 mg, and 250 mg. In some embodiments, the mixture of triacylglycerols can include glyceryl esters of oleic acid, linoleic acid, palmitic acid, stearic acid, and arachidic acid. In some embodiments, the mixture of triacylglycerols can include glyceryl esters of ricinoleic acid, oleic acid, linoleic acid, palmitic acid, stearic acid, and dihydroxystearic acid. In some embodiments, the mixture of triacylglycerols can include glyceryl esters of lauric acid, myristic acid, palmitic acid, oleic acid, caprylic acid, stearic acid, capric acid, caproic acid, linoleic acid, and arachidic acid. In some embodiments, the mixture of triacylglycerols can include glyceryl esters of linoleic acid, oleic acid, palmitic acid, capric acid, caprylic acid, stearic acid, and myristic acid. In some embodiments, the mixture of triacylglycerols can include glyceryl esters of linoleic acid, oleic acid, palmitic acid, stearic acid, and myristic acid. In some embodiments, the mixture of triacylglycerols can include glyceryl esters of linoleic acid, oleic acid, palmitic acid, stearic acid, alpha-linolenic acid, and palmitoleic acid. In some embodiments, the mixture of triacylglycerols can include glyceryl esters of oleic acid, palmitic acid, linoleic acid, stearic acid, myristic acid, and arachidic acid. In some embodiments, the mixture of triacylglycerols can include glyceryl esters of oleic acid, linoleic acid, palmitic acid, stearic acid, behenic acid, arachidic acid, and lignoceric acid. In some embodiments, the mixture of triacylglycerols can include glyceryl esters of erucic acid, oleic acid, gondonic acid, linoleic acid, alpha-linolenic acid, palmitic acid, and stearic acid. In some embodiments, the mixture of triacylglycerols can include glyceryl esters of oleic acid, linoleic acid, alpha-linolenic acid, palmitic acid, gondonic acid, and stearic acid. In some embodiments, the mixture of triacylglycerols can include glyceryl esters of linoleic acid, oleic acid, palmitic acid, and stearic acid. In some embodiments, the mixture of triacylglycerols can include glyceryl esters of linoleic acid, oleic acid, palmitic acid, linolenic acid, and stearic acid. In some embodiments, the mixture of triacylglycerols can include glyceryl esters of linoleic acid, oleic acid, palmitic acid, stearic acid, arachidic acid, and behenic acid.

Some embodiments disclosed herein relate to a pharmaceutical composition that includes Compound I and at least one pharmaceutically acceptable carrier, where the at least one pharmaceutically acceptable carrier can include a mixture of triacylglycerols. In some embodiments, the mixture of triacylglycerols can include glyceryl esters having a fatty acid content of 40-50% oleic acid, 35-45% linoleic acid, 7-9% palmitic acid, 4-5% stearic acid, and 0.4-1% arachidic acid. In some embodiments, the mixture of triacylglycerols can include glyceryl esters having a fatty acid content of 85-95% ricinoleic acid, 2-8% oleic acid, 1-6% linoleic acid, 0.5-3% palmitic acid, 0.5-1% stearic acid, and 0.3-0.7% dihydroxystearic acid. In some embodiments, the mixture of triacylglycerols can include glyceryl esters having a fatty acid content of 44-52% lauric acid, 13-19% myristic acid, 8-11% palmitic acid, 6-10% capric acid, 5-8% oleic acid, 5-9% caprylic acid, 1-3% stearic acid, 0-1% linoleic acid, 0-0.8% caproic acid, and 0-0.5% arachidic acid. In some embodiments, the mixture of triacylglycerols can include glyceryl esters having a fatty acid content of 34-62% linoleic acid, 19-49% oleic acid, 8-12% palmitic acid, 7% capric acid, 4% caprylic acid, 2-5% stearic acid, and 0.2-1% myristic acid. In some embodiments, the mixture of triacylglycerols can include glyceryl esters having a fatty acid content of 40-63% linoleic acid, 13-44% oleic acid, 17-29% palmitic acid, 1-4% stearic acid, 0.5-2% myristic acid, and 0.1-2% alpha-linolenic acid. In some embodiments, the mixture of triacylglycerols can include glyceryl esters having a fatty acid content of 60-75% linoleic acid, 12-25% oleic acid, 6-9% palmitic acid, 3-6% stearic acid, 0-1.5% alpha-linolenic acid, and 0-1% palmitoleic acid. In some embodiments, the mixture of triacylglycerols can include glyceryl esters having a fatty acid content of 65-80% oleic acid, 7-16% palmitic acid, 4-10% linoleic acid, 1-3% stearic acid, 0.1-1% myristic acid, and 0.1-0.3% arachidic acid. In some embodiments, the mixture of triacylglycerols can include glyceryl esters having a fatty acid content of 38-52% oleic acid, 32-45% palmitic acid, 5-11% linoleic acid, 2-7% stearic acid, and 0.5-2% myristic acid. In some embodiments, the mixture of triacylglycerols can include glyceryl esters having a fatty acid content of 47-56% oleic acid, 26-33% linoleic acid, and 8-10% palmitic acid. In some embodiments, the mixture of triacylglycerols can include glyceryl esters having a fatty acid content of 41% erucic acid, 17% oleic acid, 15% gondonic acid, 13% linoleic acid, 9% alpha-linolenic acid, 4% palmitic acid, and 1.5% stearic acid. In some embodiments, the mixture of triacylglycerols can include glyceryl esters having a fatty acid content of 61-63% oleic acid, 20-21% linoleic acid, 9-11% alpha-linolenic acid, 4% palmitic acid, 2% gondonic acid, 2% stearic acid, and less than 2% erucic acid. In some embodiments, the mixture of triacylglycerols can include glyceryl esters having a fatty acid content of 73-79% linoleic acid, 13-21% oleic acid, 3-6% palmitic acid, and 1-4% stearic acid. In some embodiments, the mixture of triacylglycerols can include glyceryl esters having a fatty acid content of 43-56% linoleic acid, 22-34% oleic acid, 7-11% palmitic acid, 5-11% linolenic acid, and 2-6% stearic acid. In some embodiments, the mixture of triacylglycerols can include glyceryl esters having a fatty acid content of 44-75% linoleic acid, 14-35% oleic acid, 3-6% palmitic acid, 1-3% stearic acid, 0.6-4% arachidic acid, and 1% behenic acid.

Some embodiments provided herein relate to a pharmaceutical formulation that comprises Compound I and at least one pharmaceutically acceptable carrier. In some embodiments, Compound I is 5-hydroxy-2-methylnaphthalene-1,4-dione, and the at least one pharmaceutically acceptable carrier comprises a mixture of triacylglycerols, wherein the triacylglycerols comprise glyceryl esters of one or more fatty acids selected from the group consisting of caproic acid, caprylic acid, capric acid, lauric acid, myristic acid, palmitic acid, stearic acid, oleic acid, linolenic acid, linoleic acid, arachidic acid, ricinoleic acid, dihydroxystearic acid, behenic acid, ligoceric acid, erucic acid, and gondoic acid and/or combinations thereof; and wherein the formulation is formulated as an emulsion. In some embodiments, Compound I is present in an amount of 0.05 mg/mL to 600 mg/mL, such as 0.05 mg/ml, 0.1 mg/ml, 1.0, mg/ml, 5.0 mg/ml, 10.0 mg/ml, 20 mg/ml, 30 mg/ml, 40 mg/ml, 50 mg/ml, 60 mg/ml, 70 mg/ml, 80 mg/ml, 90 mg/ml, 100 mg/ml, 150 mg/ml, 200 mg/ml, 250 mg/ml, 300 mg/ml, 350 mg/ml, 400 mg/ml, 450 mg/ml, 500 mg/ml, 550 mg/ml, or 600 mg/ml, or an amount that is within a range defined by ay two of the aforementioned amounts. In some embodiments, the emulsion is a microemulsion or a nanoemulsion. In some embodiments, the emulsion has components between 60 nm and 600 nm in diameter, such as 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 250 nm, 300 nm, 350 nm, 400 nm, 450 m, 500 nm, 550 nm, or 600 nm and/or a size that is within a range defined by any two of the aforementioned sizes. In some embodiments, the emulsion is 133 nm in diameter. In some embodiments, the pharmaceutically acceptable carrier is present in an amount ranging from 5% to 40% w/w, such as 5%, 6%, 7%, 8%, 9%, 10%, 11%, 12%, 13%, 14%, 15%, 16%, 17%, 18%, 19%, 20%, 25%, 30%, 35%, or 40% and/or an amount that is within a range defined by any two of the aforementioned percentages. In some embodiments, the pharmaceutically acceptable carrier comprises oleic acid in an amount of 10% w/w. In some embodiments, the pharmaceutically acceptable carrier comprises caprylocarpoyl polyoxyl-8 glycerides/polyoxyl castor oil (1:1) and propylene glycol monocaprylate in a ratio ranging from 0.5 to 2.5, such as 0.5, 0.75, 1.0, 1.25, 1.5, 1.75, 2.0, 2.25, or 2.5 or within a range defined by any two of the aforementioned values. In some embodiments, the pharmaceutically acceptable carrier comprises caprylocarpoyl polyoxyl-8 glycerides/polyoxyl castor oil (1:1) and propylene glycol monocaprylate in a ratio of 1.35. In some embodiments, the formulation remains stable for a period ranging from 1 day to 1 year. In some embodiments, the formulation reduces or inhibits proliferation of prostate carcinoma cells.

Some embodiments provided herein relate to a method of reducing or inhibiting proliferation of prostate carcinoma cells. In some embodiments, the method comprises administering to a subject at risk for reduced prostate cancer survival a pharmaceutical formulation described herein, including a pharmaceutical composition as described herein or a pharmaceutical composition that is formulated as an emulsion, as described herein.

Some embodiments provided herein relate to a method of making a pharmaceutical formulation as described herein, wherein the pharmaceutical formulation is formulated as an emulsion. In some embodiments, the method comprises mixing a saturated amount of Compound I with a pharmaceutically acceptable carrier and homogenizing the mixture to generate an emulsion of a pharmaceutically acceptable carrier comprising Compound I. In some embodiments, Compound I is added in an amount ranging from 0.05 mg/mL to 600 mg/mL, such as 0.05 mg/ml, 0.1 mg/ml, 1.0, mg/ml, 5.0 mg/ml, 10.0 mg/ml, 20 mg/ml, 30 mg/ml, 40 mg/ml, 50 mg/ml, 60 mg/ml, 70 mg/ml, 80 mg/ml, 90 mg/ml, 100 mg/ml, 150 mg/ml, 200 mg/ml, 250 mg/ml, 300 mg/ml, 350 mg/ml, 400 mg/ml, 450 mg/ml, 500 mg/ml, 550 mg/ml, or 600 mg/ml, or an amount that is within a range defined by ay two of the aforementioned amounts. In some embodiments, the mixture is homogenized at 8,000 rpm to 60,000 rpm, such as at 30,000 rpm. In some embodiments, the mixture is homogenized with high-pressure homogenization. In some embodiments, high-pressure homogenization is performed at 2000 psi to 10000 psi, such as at 5000 psi.

Some embodiments provided herein relate to a composition comprising 5-hydroxy-2-methylnaphthalene-1,4-dione, wherein said 5-hydroxy-2-methylnaphthalene-1,4-dione is present in said composition in an oleic acid-based microemulsion having a Z-average of less than 150 nm but not zero and polysorbate 80 at 3.5% (w/w), optionally, including an androgen deprivation agent that reduces the amount of androgen in the subject. In some embodiments, said 5-hydroxy-2-methylnaphthalene-1,4-dione is present in an amount of 4 μM. In some embodiments, the oleic acid is present in an amount of 10% (w/w). In some embodiments, the androgen deprivation agent is selected from the group consisting of cyproterone acetate, abiraterone, finasteride, flutamide, nilutamide, bicalutamide, diethylstilbestrol (DES), megestrol acetate, fosfestrol, estamustine phosphate, leuprolide, triptorelin, goserelin, histrelin, buserelin, abarelix, degarelix, orteronel, VT-464, enzalutamide, ARN-509, vinclozolin, galeterone, ketoconazole, L-39, aminoglutethimide, prochloraz, dutasteride, izonsteride, turosteride, epristeride, genisterin, gossypol, equol, 18β-glycyrrhetinic acid, altraric acid, N-butylbenzene-sulfonamide, 3,3′-diindolylmethane, deslorelin, nafarelin, cetrorelix, and ganirelix. In some embodiments, the microemulsion is generated by high-speed and/or high-pressure homogenization. In some embodiments, the composition remains stable for a period ranging from 1 day to 1 year. In further embodiments is provided the composition as described herein, for use in treating or ameliorating prostate cancer in the presence or absence of an androgen deprivation agent that reduces the amount of androgen in the subject.

Some embodiments disclosed herein relate to the following enumerated alternatives.

1. A method for increasing survival of a subject or treating a subject suffering from prostate cancer, comprising:
identifying a subject at risk for reduced prostate cancer survival or a subject having prostate cancer;
administering to the subject a pharmaceutical composition comprising Compound I and at least one pharmaceutically acceptable carrier;
wherein Compound I is 5-hydroxy-2-methylnaphthalene-1,4-dione;
wherein the at least one pharmaceutically acceptable carrier comprises a mixture of triacylglycerols, wherein the triacylglycerols comprise glyceryl esters of one or more fatty acids selected from the group consisting of caproic acid, caprylic acid, capric acid, lauric acid, myristic acid, palmitic acid, stearic acid, oleic acid, linolenic acid, linoleic acid, arachidic acid, ricinoleic acid, dihydroxystearic acid, behenic acid, ligoceric acid, erucic acid, and gondoic acid;
wherein the pharmaceutically acceptable carrier is a liquid at 25° C.;
wherein Compound I is dissolved in the pharmaceutically acceptable carrier at a concentration between 0.05 to 100 mg of Compound I per mL of pharmaceutically acceptable carrier; and
wherein the administration of said pharmaceutical composition results in survival of the subject for more than 1 month.

2. The method of alternative 1, wherein when Compound I is dissolved in the pharmaceutically acceptable carrier at 25° C., the resulting mixture is a liquid having a viscosity between 20-250 milliPascal seconds (mPa·s).

3. The method of alternative 1, wherein when Compound I is dissolved in the pharmaceutically acceptable carrier at 37° C., the resulting mixture is a liquid having a viscosity between 20-250 milliPascal seconds (mPa·s).

4. The method of any one of alternatives 1-3, wherein the subject at risk for reduced prostate cancer survival has a ruptured or broken prostate capsule, or metastatic prostate cancer.

5. The method of any one of alternatives 1-4, wherein the subject at risk for reduced prostate cancer survival has previously undergone surgical castration.

6. The method of any one of alternatives 1-5, further comprising administering to the subject androgen deprivation therapy that reduces the amount or production or synthesis of androgen in the subject.

7. The method of alternative 6, wherein the androgen deprivation therapy is selected from the group consisting of abiraterone, finasteride, dutasteride, degarelix, and leuprolide.

8. The method of any one of alternatives 1-7, wherein:
the mixture of triacylglycerols comprise two or more glyceryl esters of oleic acid, linoleic acid, palmitic acid, stearic acid, and/or arachidic acid.

9. The method of alternative 8, wherein:
the mixture of triacylglycerols comprise two or more of glyceryl esters having a fatty acid content of 40-50% oleic acid, 35-45% linoleic acid, 7-9% palmitic acid, 4-5% stearic acid, and/or 0.4-1% arachidic acid.

10. The method of any one of alternatives 1-9, wherein:
the mixture of triacylglycerols comprise two or more of glyceryl esters of ricinoleic acid, oleic acid, linoleic acid, palmitic acid, stearic acid, and/or dihydroxystearic acid; or
the mixture of triacylglycerols comprise two or more of glyceryl esters of lauric acid, myristic acid, palmitic acid, oleic acid, caprylic acid, stearic acid, capric acid, caproic acid, linoleic acid, and/or arachidic acid; or
the mixture of triacylglycerols comprise two or more of glyceryl esters of linoleic acid, oleic acid, palmitic acid, capric acid, caprylic acid, stearic acid, and/or myristic acid; or
the mixture of triacylglycerols comprise two or more of glyceryl esters of linoleic acid, oleic acid, palmitic acid, stearic acid, and/or myristic acid; or
the mixture of triacylglycerols comprise two or more of glyceryl esters of linoleic acid, oleic acid, palmitic acid, stearic acid, alpha-linolenic acid, and/or palmitoleic acid; or
the mixture of triacylglycerols comprise two or more of glyceryl esters of oleic acid, palmitic acid, linoleic acid, stearic acid, myristic acid, and/or arachidic acid; or
the mixture of triacylglycerols comprise two or more of glyceryl esters of oleic acid, linoleic acid, palmitic acid, stearic acid, behenic acid, arachidic acid, and/or lignoceric acid; or
the mixture of triacylglycerols comprise two or more of glyceryl esters of erucic acid, oleic acid, gondonic acid, linoleic acid, alpha-linolenic acid, palmitic acid, and/or stearic acid; or
the mixture of triacylglycerols comprise two or more of glyceryl esters of oleic acid, linoleic acid, alpha-linolenic acid, palmitic acid, gondonic acid, and/or stearic acid; or
the mixture of triacylglycerols comprise two or more of glyceryl esters of linoleic acid, oleic acid, palmitic acid, and/or stearic acid; or
the mixture of triacylglycerols comprise two or more of glyceryl esters of linoleic acid, oleic acid, palmitic acid, linolenic acid, and/or stearic acid; or
the mixture of triacylglycerols comprise two or more glyceryl esters of linoleic acid, oleic acid, palmitic acid, stearic acid, arachidic acid, and/or behenic acid.

11. The method of alternative 10, wherein:
the mixture of triacylglycerols comprise glyceryl esters having a fatty acid content of 85-95% ricinoleic acid, 2-8% oleic acid, 1-6% linoleic acid, 0.5-3% palmitic acid, 0.5-1% stearic acid, and 0.3-0.7% dihydroxystearic acid; or
the mixture of triacylglycerols comprise glyceryl esters having a fatty acid content of 44-52% lauric acid, 13-19% myristic acid, 8-11% palmitic acid, 6-10% capric acid, 5-8% oleic acid, 5-9% caprylic acid, 1-3% stearic acid, 0-1% linoleic acid, 0-0.8% caproic acid, and 0-0.5% arachidic acid; or
the mixture of triacylglycerols comprise glyceryl esters having a fatty acid content of 34-62% linoleic acid, 19-49% oleic acid, 8-12% palmitic acid, 7% capric acid, 4% caprylic acid, 2-5% stearic acid, and 0.2-1% myristic acid; or
the mixture of triacylglycerols comprise glyceryl esters having a fatty acid content of 40-63% linoleic acid, 13-44% oleic acid, 17-29% palmitic acid, 1-4% stearic acid, 0.5-2% myristic acid, and 0.1-2% alpha-linolenic acid; or
the mixture of triacylglycerols comprise glyceryl esters having a fatty acid content of 60-75% linoleic acid, 12-25% oleic acid, 6-9% palmitic acid, 3-6% stearic acid, 0-1.5% alpha-linolenic acid, and 0-1% palmitoleic acid; or
the mixture of triacylglycerols comprise glyceryl esters having a fatty acid content of 65-80% oleic acid, 7-16% palmitic acid, 4-10% linoleic acid, 1-3% stearic acid, 0.1-1% myristic acid, and 0.1-0.3% arachidic acid; or
the mixture of triacylglycerols comprise glyceryl esters having a fatty acid content of 38-52% oleic acid, 32-45% palmitic acid, 5-11% linoleic acid, 2-7% stearic acid, and 0.5-2% myristic acid; or
the mixture of triacylglycerols comprise glyceryl esters having a fatty acid content of 47-56% oleic acid, 26-33% linoleic acid, and 8-10% palmitic acid; or
the mixture of triacylglycerols comprise glyceryl esters having a fatty acid content of 41% erucic acid, 17% oleic acid, 15% gondonic acid, 13% linoleic acid, 9% alpha-linolenic acid, 4% palmitic acid, and 1.5% stearic acid; or
the mixture of triacylglycerols comprise glyceryl esters having a fatty acid content of 61-63% oleic acid, 20-21% linoleic acid, 9-11% alpha-linolenic acid, 4% palmitic acid, 2% gondonic acid, 2% stearic acid, and less than 2% erucic acid; or
the mixture of triacylglycerols comprise glyceryl esters having a fatty acid content of 73-79% linoleic acid, 13-21% oleic acid, 3-6% palmitic acid, and 1-4% stearic acid; or
the mixture of triacylglycerols comprise glyceryl esters having a fatty acid content of 43-56% linoleic acid, 22-34% oleic acid, 7-11% palmitic acid, 5-11% linolenic acid, and 2-6% stearic acid; or
the mixture of triacylglycerols comprise glyceryl esters having a fatty acid content of 44-75% linoleic acid, 14-35% oleic acid, 3-6% palmitic acid, 1-3% stearic acid, 0.6-4% arachidic acid, and 1% behenic acid.

12. The method of any one of alternatives 1-11, wherein the amount of Compound I in the pharmaceutical composition is selected from the group consisting of 25 mg, 50 mg, 75 mg, 100 mg, 125 mg, 150 mg, 200 mg, and 250 mg.

13. The method of any one of alternatives 1-12, wherein the subject is administered a dosage amount of 1 mg of Compound I per kg of subject, and the pharmaceutical composition is formulated for oral administration to the patient.

14. A method for inhibiting, delaying, or preventing rupture of a subject's prostatic capsule in a subject suffering from prostate cancer, comprising:
administering to the subject a pharmaceutical composition comprising Compound I and at least one pharmaceutically acceptable carrier;
wherein Compound I is 5-hydroxy-2-methylnaphthalene-1,4-dione;
wherein the at least one pharmaceutically acceptable carrier comprises a mixture of triacylglycerols, wherein the triacylglycerols comprise glyceryl esters of one or more fatty acids selected from the group consisting of caproic acid, caprylic acid, capric acid, lauric acid, myristic acid, palmitic acid, stearic acid, oleic acid, linolenic acid, linoleic acid, arachidic acid, ricinoleic acid, dihydroxystearic acid, behenic acid, ligoceric acid, erucic acid, and/or gondoic acid;
wherein the pharmaceutically acceptable carrier is a liquid at 25° C.; and
wherein Compound I is dissolved in the pharmaceutically acceptable carrier at a concentration between 0.05 to 100 mg of Compound I per mL of pharmaceutically acceptable carrier.

15. The method of alternative 14, wherein when Compound I is dissolved in the pharmaceutically acceptable carrier at 25° C., the resulting mixture is a liquid having a viscosity between 20-250 milliPascal seconds (mPa·s).

16. The method of alternative 14, wherein when Compound I is dissolved in the pharmaceutically acceptable carrier at 37° C., the resulting mixture is a liquid having a viscosity between 20-250 milliPascal seconds (mPa·s).

17. The method of any one of alternatives 14-16, wherein the subject at risk for reduced prostate cancer survival has previously undergone surgical castration.

18. The method of any one of alternatives 14-17, further comprising administering to the subject androgen deprivation therapy that reduces the amount, inhibits the production or inhibits the synthesis of androgen in the subject.

19. The method of alternative 18, wherein the androgen deprivation therapy is selected from the group consisting of abiraterone, finasteride, dutasteride, degarelix, and leuprolide.

20. The method of any one of alternatives 14-19, wherein:
the mixture of triacylglycerols comprise two or more glyceryl esters of oleic acid, linoleic acid, palmitic acid, stearic acid, and/or arachidic acid.

21. The method of alternative 20, wherein:
the mixture of triacylglycerols comprise glyceryl esters having a fatty acid content of 40-50% oleic acid, 35-45% linoleic acid, 7-9% palmitic acid, 4-5% stearic acid, and 0.4-1% arachidic acid.

22. The method of any one of alternatives 14-21, wherein:
the mixture of triacylglycerols comprise two or more glyceryl esters of ricinoleic acid, oleic acid, linoleic acid, palmitic acid, stearic acid, and/or dihydroxystearic acid; or
the mixture of triacylglycerols comprise two or more glyceryl esters of lauric acid, myristic acid, palmitic acid, oleic acid, caprylic acid, stearic acid, capric acid, caproic acid, linoleic acid, and/or arachidic acid; or
the mixture of triacylglycerols comprise two or more glyceryl esters of linoleic acid, oleic acid, palmitic acid, capric acid, caprylic acid, stearic acid, and/or myristic acid; or
the mixture of triacylglycerols comprise two or more glyceryl esters of linoleic acid, oleic acid, palmitic acid, stearic acid, and/or myristic acid; or
the mixture of triacylglycerols comprise two or more glyceryl esters of linoleic acid, oleic acid, palmitic acid, stearic acid, alpha-linolenic acid, and/or palmitoleic acid; or
the mixture of triacylglycerols comprise two or more glyceryl esters of oleic acid, palmitic acid, linoleic acid, stearic acid, myristic acid, and/or arachidic acid; or
the mixture of triacylglycerols comprise two or more glyceryl esters of oleic acid, linoleic acid, palmitic acid, stearic acid, behenic acid, arachidic acid, and/or lignoceric acid; or
the mixture of triacylglycerols comprise two or more glyceryl esters of erucic acid, oleic acid, gondonic acid, linoleic acid, alpha-linolenic acid, palmitic acid, and/or stearic acid; or
the mixture of triacylglycerols comprise two or more glyceryl esters of oleic acid, linoleic acid, alpha-linolenic acid, palmitic acid, gondonic acid, and/or stearic acid; or
the mixture of triacylglycerols comprise two or more glyceryl esters of linoleic acid, oleic acid, palmitic acid, and/or stearic acid; or
the mixture of triacylglycerols comprise two or more glyceryl esters of linoleic acid, oleic acid, palmitic acid, linolenic acid, and/or stearic acid; or
the mixture of triacylglycerols comprise two or more glyceryl esters of linoleic acid, oleic acid, palmitic acid, stearic acid, arachidic acid, and/or behenic acid.

23. The method of alternative 22, wherein:
the mixture of triacylglycerols comprise glyceryl esters having a fatty acid content of 85-95% ricinoleic acid, 2-8% oleic acid, 1-6% linoleic acid, 0.5-3% palmitic acid, 0.5-1% stearic acid, and 0.3-0.7% dihydroxystearic acid; or
the mixture of triacylglycerols comprise glyceryl esters having a fatty acid content of 44-52% lauric acid, 13-19% myristic acid, 8-11% palmitic acid, 6-10% capric acid, 5-8% oleic acid, 5-9% caprylic acid, 1-3% stearic acid, 0-1% linoleic acid, 0-0.8% caproic acid, and 0-0.5% arachidic acid; or
the mixture of triacylglycerols comprise glyceryl esters having a fatty acid content of 34-62% linoleic acid, 19-49% oleic acid, 8-12% palmitic acid, 7% capric acid, 4% caprylic acid, 2-5% stearic acid, and 0.2-1% myristic acid; or
the mixture of triacylglycerols comprise glyceryl esters having a fatty acid content of 40-63% linoleic acid, 13-44% oleic acid, 17-29% palmitic acid, 1-4% stearic acid, 0.5-2% myristic acid, and 0.1-2% alpha-linolenic acid; or
the mixture of triacylglycerols comprise glyceryl esters having a fatty acid content of 60-75% linoleic acid, 12-25% oleic acid, 6-9% palmitic acid, 3-6% stearic acid, 0-1.5% alpha-linolenic acid, and 0-1% palmitoleic acid; or
the mixture of triacylglycerols comprise glyceryl esters having a fatty acid content of 65-80% oleic acid, 7-16% palmitic acid, 4-10% linoleic acid, 1-3% stearic acid, 0.1-1% myristic acid, and 0.1-0.3% arachidic acid; or
the mixture of triacylglycerols comprise glyceryl esters having a fatty acid content of 38-52% oleic acid, 32-45% palmitic acid, 5-11% linoleic acid, 2-7% stearic acid, and 0.5-2% myristic acid; or
the mixture of triacylglycerols comprise glyceryl esters having a fatty acid content of 47-56% oleic acid, 26-33% linoleic acid, and 8-10% palmitic acid; or
the mixture of triacylglycerols comprise glyceryl esters having a fatty acid content of 41% erucic acid, 17% oleic acid, 15% gondonic acid, 13% linoleic acid, 9% alpha-linolenic acid, 4% palmitic acid, and 1.5% stearic acid; or
the mixture of triacylglycerols comprise glyceryl esters having a fatty acid content of 61-63% oleic acid, 20-21% linoleic acid, 9-11% alpha-linolenic acid, 4% palmitic acid, 2% gondonic acid, 2% stearic acid, and less than 2% erucic acid; or
the mixture of triacylglycerols comprise glyceryl esters having a fatty acid content of 73-79% linoleic acid, 13-21% oleic acid, 3-6% palmitic acid, and 1-4% stearic acid; or
the mixture of triacylglycerols comprise glyceryl esters having a fatty acid content of 43-56% linoleic acid, 22-34% oleic acid, 7-11% palmitic acid, 5-11% linolenic acid, and 2-6% stearic acid; or
the mixture of triacylglycerols comprise glyceryl esters having a fatty acid content of 44-75% linoleic acid, 14-35% oleic acid, 3-6% palmitic acid, 1-3% stearic acid, 0.6-4% arachidic acid, and 1% behenic acid.

24. The method of any one of alternatives 14-23, wherein the amount of Compound I in the pharmaceutical composition is selected from the group consisting of 25 mg, 50 mg, 75 mg, 100 mg, 125 mg, 150 mg, 200 mg, and 250 mg.

25. The method of any one of alternatives 14-24, wherein the subject is administered a dosage amount of 1 mg of Compound I per kg of subject, and the pharmaceutical composition is formulated for oral administration to the patient.

26. A pharmaceutical composition comprising:
Compound I and at least one pharmaceutically acceptable carrier;
wherein Compound I is 5-hydroxy-2-methylnaphthalene-1,4-dione;
wherein the at least one pharmaceutically acceptable carrier comprises a mixture of triacylglycerols, wherein the triacylglycerols comprise glyceryl esters of one or more fatty acids selected from the group consisting of caproic acid, caprylic acid, capric acid, lauric acid, myristic acid, palmitic acid, stearic acid, oleic acid, linolenic acid, linoleic acid, arachidic acid, ricinoleic acid, dihydroxystearic acid, behenic acid, ligoceric acid, erucic acid, and gondoic acid; and
wherein the pharmaceutically acceptable carrier is a liquid at 25° C.;
wherein Compound I is dissolved in the pharmaceutically acceptable carrier at a concentration between 0.05 to 100 mg of Compound I per mL of pharmaceutically acceptable carrier.

27. The pharmaceutical composition of alternative 26, wherein when Compound I is dissolved in the pharmaceutically acceptable carrier at 25° C., the resulting mixture is a liquid having a viscosity between 20-250 milliPascal seconds (mPa·s).

28. The pharmaceutical composition of alternative 26, wherein when Compound I is dissolved in the pharmaceutically acceptable carrier at 37° C., the resulting mixture is a liquid having a viscosity between 20-250 milliPascal seconds (mPa·s).

29. The pharmaceutical composition of any one of alternatives 26-28, wherein:
the mixture of triacylglycerols comprise two or more glyceryl esters of oleic acid, linoleic acid, palmitic acid, stearic acid, and/or arachidic acid.

30. The pharmaceutical composition of alternative 29, wherein:
the mixture of triacylglycerols comprise glyceryl esters having a fatty acid content of 40-50% oleic acid, 35-45% linoleic acid, 7-9% palmitic acid, 4-5% stearic acid, and 0.4-1% arachidic acid.

31. The pharmaceutical composition of any one of alternatives 26-30, wherein:
the mixture of triacylglycerols comprise two or more glyceryl esters of ricinoleic acid, oleic acid, linoleic acid, palmitic acid, stearic acid, and/or dihydroxystearic acid; or
the mixture of triacylglycerols comprise two or more glyceryl esters of lauric acid, myristic acid, palmitic acid, oleic acid, caprylic acid, stearic acid, capric acid, caproic acid, linoleic acid, and/or arachidic acid; or
the mixture of triacylglycerols comprise two or more glyceryl esters of linoleic acid, oleic acid, palmitic acid, capric acid, caprylic acid, stearic acid, and/or myristic acid; or
the mixture of triacylglycerols comprise two or more glyceryl esters of linoleic acid, oleic acid, palmitic acid, stearic acid, and/or myristic acid; or
the mixture of triacylglycerols comprise two or more glyceryl esters of linoleic acid, oleic acid, palmitic acid, stearic acid, alpha-linolenic acid, and/or palmitoleic acid; or
the mixture of triacylglycerols comprise two or more glyceryl esters of oleic acid, palmitic acid, linoleic acid, stearic acid, myristic acid, and/or arachidic acid; or
the mixture of triacylglycerols comprise two or more glyceryl esters of oleic acid, linoleic acid, palmitic acid, stearic acid, behenic acid, arachidic acid, and/or lignoceric acid; or
the mixture of triacylglycerols comprise two or more glyceryl esters of erucic acid, oleic acid, gondonic acid, linoleic acid, alpha-linolenic acid, palmitic acid, and/or stearic acid; or
the mixture of triacylglycerols comprise two or more glyceryl esters of oleic acid, linoleic acid, alpha-linolenic acid, palmitic acid, gondonic acid, and/or stearic acid; or
the mixture of triacylglycerols comprise two or more glyceryl esters of linoleic acid, oleic acid, palmitic acid, and/or stearic acid; or
the mixture of triacylglycerols comprise two or more glyceryl esters of linoleic acid, oleic acid, palmitic acid, linolenic acid, and/or stearic acid; or
the mixture of triacylglycerols comprise two or more glyceryl esters of linoleic acid, oleic acid, palmitic acid, stearic acid, arachidic acid, and/or behenic acid.

32. The pharmaceutical composition of alternative 31, wherein:
the mixture of triacylglycerols comprise glyceryl esters having a fatty acid content of 85-95% ricinoleic acid, 2-8% oleic acid, 1-6% linoleic acid, 0.5-3% palmitic acid, 0.5-1% stearic acid, and 0.3-0.7% dihydroxystearic acid; or
the mixture of triacylglycerols comprise glyceryl esters having a fatty acid content of 44-52% lauric acid, 13-19% myristic acid, 8-11% palmitic acid, 6-10% capric acid, 5-8% oleic acid, 5-9% caprylic acid, 1-3% stearic acid, 0-1% linoleic acid, 0-0.8% caproic acid, and 0-0.5% arachidic acid; or
the mixture of triacylglycerols comprise glyceryl esters having a fatty acid content of 34-62% linoleic acid, 19-49% oleic acid, 8-12% palmitic acid, 7% capric acid, 4% caprylic acid, 2-5% stearic acid, and 0.2-1% myristic acid; or
the mixture of triacylglycerols comprise glyceryl esters having a fatty acid content of 40-63% linoleic acid, 13-44% oleic acid, 17-29% palmitic acid, 1-4% stearic acid, 0.5-2% myristic acid, and 0.1-2% alpha-linolenic acid; or
the mixture of triacylglycerols comprise glyceryl esters having a fatty acid content of 60-75% linoleic acid, 12-25% oleic acid, 6-9% palmitic acid, 3-6% stearic acid, 0-1.5% alpha-linolenic acid, and 0-1% palmitoleic acid; or
the mixture of triacylglycerols comprise glyceryl esters having a fatty acid content of 65-80% oleic acid, 7-16% palmitic acid, 4-10% linoleic acid, 1-3% stearic acid, 0.1-1% myristic acid, and 0.1-0.3% arachidic acid; or
the mixture of triacylglycerols comprise glyceryl esters having a fatty acid content of 38-52% oleic acid, 32-45% palmitic acid, 5-11% linoleic acid, 2-7% stearic acid, and 0.5-2% myristic acid; or
the mixture of triacylglycerols comprise glyceryl esters having a fatty acid content of 47-56% oleic acid, 26-33% linoleic acid, and 8-10% palmitic acid; or
the mixture of triacylglycerols comprise glyceryl esters having a fatty acid content of 41% erucic acid, 17% oleic acid, 15% gondonic acid, 13% linoleic acid, 9% alpha-linolenic acid, 4% palmitic acid, and 1.5% stearic acid; or
the mixture of triacylglycerols comprise glyceryl esters having a fatty acid content of 61-63% oleic acid, 20-21% linoleic acid, 9-11% alpha-linolenic acid, 4% palmitic acid, 2% gondonic acid, 2% stearic acid, and less than 2% erucic acid; or
the mixture of triacylglycerols comprise glyceryl esters having a fatty acid content of 73-79% linoleic acid, 13-21% oleic acid, 3-6% palmitic acid, and 1-4% stearic acid; or
the mixture of triacylglycerols comprise glyceryl esters having a fatty acid content of 43-56% linoleic acid, 22-34% oleic acid, 7-11% palmitic acid, 5-11% linolenic acid, and 2-6% stearic acid; or
the mixture of triacylglycerols comprise glyceryl esters having a fatty acid content of 44-75% linoleic acid, 14-35% oleic acid, 3-6% palmitic acid, 1-3% stearic acid, 0.6-4% arachidic acid, and 1% behenic acid.

33. The pharmaceutical composition of any one of alternatives 26-32, wherein the amount of Compound I in the pharmaceutical composition is selected from the group consisting of 25 mg, 50 mg, 75 mg, 100 mg, 125 mg, 150 mg, 200 mg, and 250 mg.

34. A pharmaceutical formulation comprising:
Compound I and at least one pharmaceutically acceptable carrier;
wherein Compound I is 5-hydroxy-2-methylnaphthalene-1,4-dione;
wherein the at least one pharmaceutically acceptable carrier comprises a mixture of triacylglycerols, wherein the triacylglycerols comprise glyceryl esters of one or more fatty acids selected from the group consisting of caproic acid, caprylic acid, capric acid, lauric acid, myristic acid, palmitic acid, stearic acid, oleic acid, linolenic acid, linoleic acid, arachidic acid, ricinoleic acid, dihydroxystearic acid, behenic acid, ligoceric acid, erucic acid, and gondoic acid; and
wherein the formulation is formulated as an emulsion.

35. The pharmaceutical formulation of alternative 34, wherein Compound I is present in an amount of 0.05 mg/mL to 600 mg/mL.

36. The pharmaceutical formulation of any one of alternatives 34-35, wherein the emulsion is a microemulsion or a nanoemulsion.

37. The pharmaceutical formulation of any one of alternatives 34-36, wherein the emulsion is between 60 nm and 600 nm in diameter.

38. The pharmaceutical formulation of any one of alternatives 34-37, wherein the emulsion is 133 nm in diameter.

39. The pharmaceutical formulation of any one of alternatives 34-38, wherein the pharmaceutically acceptable carrier is present in an amount ranging from 5% to 40% w/w.

40. The pharmaceutical formulation of any one of alternatives 34-39, wherein the pharmaceutically acceptable carrier comprises oleic acid in an amount of 10% w/w.

41. The pharmaceutical formulation of any one of alternatives 34-39, wherein the pharmaceutically acceptable carrier comprises caprylocarpoyl polyoxyl-8 glycerides/polyoxyl castor oil (1:1) and propylene glycol monocaprylate in a ratio ranging from 0.5 to 2.5.

42. The pharmaceutical formulation of alternative 41, wherein the pharmaceutically acceptable carrier comprises caprylocarpoyl polyoxyl-8 glycerides/polyoxyl castor oil (1:1) and propylene glycol monocaprylate in a ratio of 1.35.

43. The pharmaceutical formulation of any one of alternatives 34-42, wherein the formulation remains stable for a period ranging from 1 day to 1 year.

44. The pharmaceutical formulation of any one of alternatives 34-43, wherein the formulation reduces proliferation of prostate carcinoma cells.

45. A method of reducing or inhibiting proliferation of prostate carcinoma cells or treating prostate cancer, comprising administering to a subject at risk for reduced prostate cancer survival a formulation of any one of alternatives 26-44.

46. A method of making a formulation of any one of alternatives 34-44, the method comprising:
mixing a saturated amount of Compound I with a pharmaceutically acceptable carrier; and
homogenizing the mixture to generate an emulsions of a pharmaceutically acceptable carrier comprising Compound I.

47. The method of alternative 46, Compound I is added in an amount ranging from 0.05 mg/mL to 600 mg/mL.

48. The method of any one of alternatives 46-47, wherein the mixture is homogenized at 8,000 rpm to 60,000 rpm, such as at 30,000 rpm.

49. The method of any one of alternatives 46-48, wherein the mixture is homogenized with high-pressure homogenization.

50. The method of alternative 49, wherein high-pressure homogenization is performed at 2000 psi to 10000 psi, such as at 5000 psi.

51. A composition comprising 5-hydroxy-2-methylnaphthalene-1,4-dione, wherein said 5-hydroxy-2-methylnaphthalene-1,4-dione is present in said composition in an oleic acid-based microemulsion having a Z-average of less than 150 nm but not zero and polysorbate 80 at 3.5% (w/w), optionally, including an androgen deprivation agent that reduces the amount of androgen in the subject.

52. The composition of alternative 51, wherein 5-hydroxy-2-methylnaphthalene-1,4-dione is present in an amount of 4 μM.

53. The composition of any one of alternatives 51-52, wherein oleic acid is present in an amount of 10% (w/w).

54. The composition of any one of alternatives 51-53, wherein the androgen deprivation agent is selected from the group consisting of cyproterone acetate, abiraterone, finasteride, flutamide, nilutamide, bicalutamide, diethylstilbestrol (DES), megestrol acetate, fosfestrol, estamustine phosphate, leuprolide, triptorelin, goserelin, histrelin, buserelin, abarelix, degarelix, orteronel, VT-464, enzalutamide, ARN-509, vinclozolin, galeterone, ketoconazole, L-39, aminoglutethimide, prochloraz, dutasteride, izonsteride, turosteride, epristeride, genisterin, gossypol, equol, 18β-glycyrrhetinic acid, altraric acid, N-butylbenzene-sulfonamide, 3,3′-diindolylmethane, deslorelin, nafarelin, cetrorelix, and ganirelix.

55. The composition of any one of alternatives 51-54, wherein the microemulsion is generated by high-speed and/or high-pressure homogenization.

56. The composition of any one of alternatives 51-55, wherein the composition remains stable for a period ranging from 1 day to 1 year.

57. The composition of any one of alternatives 51-56 for use in treating or ameliorating prostate cancer in the presence or absence of an androgen deprivation agent that reduces the amount of androgen in the subject.

### BRIEF DESCRIPTION OF THE DRAWINGS

FIG. 1 illustrates a typical steroid/androgen synthesis pathway.

FIG. 2A illustrates spectrophotometric determination of the concentration of Compound I in solution. As shown in FIG. 2B, peaks at wavelengths of 262 nm and 410 nm can be used for determination of the concentration of Compound I.

FIG. 3A illustrates in vivo effects of a pharmaceutical composition containing Compound I and pharmaceutically acceptable Carrier A administered orally at dosages of 1 mg/kg (open circles), 3 mg/kg (closed triangles), and 10 mg/kg (open triangles) in combination with castration on tumor size. FIG. 3A also illustrates in vivo effects of a formulation of Compound I in PEG administered via intra-peritoneal injection (i.p.) at a dose of 1 mg/kg in combination with castration on tumor size (solid circles).

FIG. 3B illustrates in vivo effects of a pharmaceutical composition containing Compound I and pharmaceutically acceptable Carrier A administered orally at dosages of 0.1 mg/kg (open circles), 0.3 mg/kg (closed triangles), and 1 mg/kg (open triangles) in combination with castration on tumor size, as compared with castration alone (solid circles).

FIG. 4A illustrates in vivo effects of either castration alone (dashed lines) or administering a pharmaceutical composition containing Compound I and pharmaceutically acceptable Carrier A orally in combination with castration (solid lines) on tumor size in six cohorts of mice. FIG. 4B illustrates in vivo effects of either castration alone (solid circles) or administering a formulation of Compound I in Carrier A orally in combination with castration (open circles) on tumor size in a compilation of 52 mice.

FIG. 5A illustrates the results from in vivo prostate cancer survival studies using PTEN-P2 cancer cells. The right-hand side of FIG. 5A illustrates the survival results from administering a pharmaceutical composition containing Compound I and pharmaceutically acceptable Carrier A orally in combination with castration (solid line) as compared to castration alone (dashed line). The left-hand side of FIG. 5A illustrates the survival results from the administration of a pharmaceutical composition containing Compound I and pharmaceutically acceptable Carrier A alone (solid line) and castration alone (dashed line).

FIG. 5B illustrates the results from in vivo prostate cancer survival studies using PTEN-P2 cancer cells. The top plot in FIG. 5B illustrates the survival results from administering a pharmaceutical composition containing Compound I and pharmaceutically acceptable Carrier A orally in combination with castration (solid line) as compared with no treatment (dashed line). The bottom-left plot in FIG. 5B illustrates the survival results from treatment with a pharmaceutical composition containing Compound I and pharmaceutically acceptable Carrier A alone (solid line) as compared to no treatment (dashed line). The bottom-right plot in FIG. 5B illustrates the survival results from castration alone (solid line) as compared to no treatment (dashed line).

FIG. 6 illustrates the survival results in vivo prostate cancer survival studies using TRAMP-2 cancer cells. FIG. 6 illustrates the survival results from administering a pharmaceutical composition containing Compound I and pharmaceutically acceptable Carrier A orally in combination with castration (solid line) as compared with castration alone (dashed line).

FIG. 7A shows the in vivo effects of degarelix alone (closed circles), castration alone (open circles), administering a pharmaceutical composition containing Compound I and pharmaceutically acceptable Carrier A orally in combination with degarelix (closed triangles), and administering a pharmaceutical composition containing Compound I and pharmaceutically acceptable Carrier A orally in combination with castration (open triangles) on tumor size.

FIG. 7B shows the in vivo effects of abiraterone/prednisone alone (open circles), castration alone (closed triangles), administering a pharmaceutical composition containing Compound I and pharmaceutically acceptable Carrier A orally in combination with abiraterone/prednisone (closed circles), and administering a pharmaceutical composition containing Compound I and pharmaceutically acceptable Carrier A orally in combination with castration (open triangles) on tumor size.

FIG. 7C shows the in vivo effects of orteronel alone (closed circles), castration alone (close triangles), administering a pharmaceutical composition containing Compound I and pharmaceutically acceptable Carrier A orally in combination with orteronel (open circles), and administering a pharmaceutical composition containing Compound I and pharmaceutically acceptable Carrier A orally in combination with castration (open triangles) on tumor size.

FIG. 7D shows the in vivo effects of dutasteride alone (open circles), castration alone (close triangles), administering a pharmaceutical composition containing Compound I and pharmaceutically acceptable Carrier A orally in combination with dutasteride (closed circles), and administering a pharmaceutical composition containing Compound I and pharmaceutically acceptable Carrier A orally in combination with castration (open triangles) on tumor size.

FIG. 7E shows the in vivo effects of administering dutasteride alone as compared to a pharmaceutical composition containing Compound I and pharmaceutically acceptable Carrier A orally in combination with dutasteride on tumor size using several different dosages of dutasteride.

FIG. 8 shows the in vivo effects of Lupron alone (open circles) as compared to administering a pharmaceutical composition containing Compound I and pharmaceutically acceptable Carrier A orally in combination with leuprolide (closed squares) on tumor size.

FIG. 9A shows the in vivo effects of no treatment (closed circles), bicalutamide alone (open circles), and administering a pharmaceutical composition containing Compound I and pharmaceutically acceptable Carrier A orally in combination with bicalutamide (closed triangles) on tumor size.

FIG. 9B shows the in vivo effects of castration alone (open circles), administering a pharmaceutical composition containing Compound I and pharmaceutically acceptable Carrier A orally in combination with castration (closed triangles), enzalutamide alone (open triangles), and administering a pharmaceutical composition containing Compound I and pharmaceutically acceptable Carrier A orally in combination with enzalutamide (closed squares) on tumor size.

FIG. 10 shows the in vivo effects of no treatment (closed circles), castration alone (open circles), administering a pharmaceutical composition containing Compound I and pharmaceutically acceptable Carrier A orally alone (closed triangles), and administering a pharmaceutical composition containing Compound I and pharmaceutically acceptable Carrier A orally in combination with castration (open triangles) on tumor size for castration resistant prostate cancer (CRPC).

FIG. 11 shows the change in dynamic viscosity at different temperatures for pharmaceutically acceptable Carrier A alone (open circles) and a pharmaceutical composition containing Compound I and pharmaceutically acceptable Carrier A (closed triangles).

FIG. 12 illustrates the solubility of a pharmaceutical composition containing Compound I in mg/mL in the following triglycerides as a function of the aliphatic chain length: triacetin (C2), tributyrin (C4), tricaproin (C6), captex 8000 (C8), tricaprin (C10), and glyceryl trioleate (C18).

FIG. 13 depicts a pseudoternary phase diagram of propylene glycol monocaprylate, caprylocarpoyl polyoxyl-8 glycerides/polyoxyl castor oil (1:1), and water at 25° C. The shadowed area indicates o/w nanoemulsion domain.

FIG. 14 depicts the visual appearance of a series of aqueous dispersions with different fractions of propylene glycol monocaprylate in surfactant-cosurfactant in oil mixtures (S-CoS/O).

FIG. 15A-15E depict plots showing autocorrelation function and particle size distribution of propylene glycol monocaprylate-caprylocarpoyl polyoxyl-8 glycerides/polyoxyl castor oil (1:1) in water at different S-CoS/O (w/w) ratios, including at ratios of 0.52 and 0.67 (FIG. 15A), 0.82 and 0.97 (FIG. 15B), 1.09 and 1.35 (FIG. 15C), 1.51 and 1.72 (FIG. 15D), and 2.05 (FIG. 15E).

FIG. 16 depicts a plot of intensity estimated hydrodynamic radius and polydispersity index (PDI—inset) of propylene glycol monocaprylate-(caprylocarpoyl polyoxyl-8 glycerides/polyoxyl castor oil) (1:1) at different S-CoS/0 ratios ranging from 0.67 to 2.05.

FIG. 17 shows time-dependent intensity based particle size distribution profiles of propylene glycol monocaprylate-(caprylocarpoyl polyoxyl-8 glycerides/polyoxyl castor oil) (1:1) at different S-CoS/O ratios from 0.67 to 2.05.

FIG. 18A depicts the visual appearance of a series of propylene glycol monocaprylate-(caprylocarpoyl polyoxyl-8 glycerides/polyoxyl castor oil) nanoemulsions loaded with increasing amounts of Compound I (from 1% to 8% w/w) at 25° C. FIG. 18B depicts time dependent hydrodynamic radius in nanometers of propylene glycol monocaprylate-(caprylocarpoyl polyoxyl-8 glycerides/polyoxyl castor oil) nanoemulsions loaded with increasing amounts of Compound I at 25° C.

FIG. 19 depicts plots showing autocorrelation function, intensity based particle size distribution, and zeta potential profiles of control microemulsions of oleic acid/polysorbate 80 (top row) and oleic acid/polysorbate 80 microemulsions loaded with Compound I (bottom row).

FIGS. 20A-20C depict plots showing autocorrelation function and intensity based particle size distribution of microemulsions of oleic acid/polysorbate 80 loaded with Compound I, with various w/w % of polysorbate. FIG. 20A shows 4, 3.5, and 3 w/w % polysorbate, FIG. 20B shows 2.5, 2 and 1.5 w/w % polysorbate, and FIG. 20C shows 1, 0.5, and 0.25 w/w % polysorbate.

FIG. 21 depicts a plot showing the z-average of microemulsions of oleic acid/polysorbate 80 loaded with Compound I, with various w/w % of polysorbate.

FIGS. 22A and 22B depict time-dependent intensity based particle size distribution profiles of oleic acid/polysorbate 80 based microemulsions with Compound I at different polysorbate percentages, from 4.0 to 0.25 w/w %.

FIG. 23 depicts a time dependent stability profile of hydrodynamic radius in nanometers of oleic acid-based microemulsions without Compound I (ME—control) or with Compound I (ME—Compound I) at 25° C.

FIG. 24 depicts the cytotoxicity of Compound I, microemulsions with Compound I, or empty microemulsions toward P2-PTEN cells. For the control (ME—control), the cells were exposed to the same concentration of microemulsion. P2-PTEN cells were incubated with increasing dilutions of formulations for 24 hours and then cytotoxicity was determined by CellTiter96™ AQueous on solution cell proliferation assay (MTS).

FIG. 25 depicts micrographs of P2-PTEN cells exposed for 24 hours to: untreated control (panel A); Compound I alone (4 μM, panel B); oleic acid microemulsion control without Compound I (panel C); and oleic acid microemulsion loaded with Compound I (4 μM, panel D).

### DETAILED DESCRIPTION

### I. Definitions

Unless defined otherwise, all technical and scientific terms used herein have the same meaning as is commonly understood by one of ordinary skill in the art. All patents, applications, published applications and other publications referenced herein are expressly incorporated by reference in their entireties unless stated otherwise. In the event that there are a plurality of definitions for a term herein, those in this section prevail unless stated otherwise.

The term “Compound I” refers to 5-hydroxy-2-methylnaphthalene-1,4-dione, and pharmaceutically acceptable salts thereof. 5-hydroxy-2-methylnaphthalene-1,4-dione may also be referred to as 1,4-dihydro-1,4-dioxo-5-hydroxy-2-methylnaphthalene or 5-hydroxyl-2-methyl-1,4-naphthoquinone.

The term “pharmaceutically acceptable salt” refers to a salt of a compound that does not cause significant irritation to an organism to which it is administered and does not abrogate the biological activity and properties of the compound. In some alternatives, the salt is an acid addition salt of the compound. Pharmaceutical salts can be obtained by reacting a compound with inorganic acids such as hydrochloric acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

The term “pharmaceutical composition” refers to a mixture of a compound disclosed herein with other chemical components, such as pharmaceutically acceptable carriers or excipients. The pharmaceutical composition facilitates administration of the compound to an organism.

As used herein, a “pharmaceutically acceptable carrier” refers to a substance, not itself a therapeutic agent, that may facilitate the incorporation of a compound into cells or tissues. The carrier may be a liquid for the dissolution of a compound to be administered by ingestion. The carrier may be a vehicle for delivery of a therapeutic agent to a subject. The carrier may improve the stability, handling, or storage properties of a therapeutic agent. The carrier may facilitate formation of a dose unit of a composition into a discrete article such as a capsule, tablet, film coated tablet, caplet, gel cap, pill pellet, bead, and the like suitable for oral administration to a subject.

As used herein, “pharmaceutically acceptable Carrier A” refers to a mixture of triacylglycerols having long chain saturated or unsaturated fatty acids comprising oleic acid, linoleic acid, palmitic acid, stearic acid, and arachidic acid. Carrier A may comprise a mixture of triacylglycerols, where the mixture of triacylglycerols have a fatty acid content comprising 40-50% oleic acid, 35-45% linoleic acid, 7-9% palmitic acid, 4-5% stearic acid, and 0.4-1% arachidic acid. Carrier A comprises a mixture of triacylglycerols obtained from the seeds of Sesamum indicum L., Pedaliaceae.

As used herein, when a mixture of triacylglycerols is “obtained” from a given source, the mixture may have been extracted from the source by, for example, hot water flotation, bridge presses, ram presses, the ghani process, usage of an expeller press, oil extraction, chemical solvent extraction, milling, grinding, steaming, boiling, sun-drying, and cold pressing. The “obtained” mixture of triacylglycerols can include mixtures that have been refined through one or more processes, such as solvent extraction, neutralization, bleaching, and steam stripping (distillative neutralization).

As used herein, a “pharmaceutically acceptable excipient” refers to an inert substance that is added to a pharmaceutical composition to provide, without limitation, bulk, consistency, stability, binding ability, lubrication, or disintegrating ability to the composition. A “diluent” is a type of excipient.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some alternative, the subject is human.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy.

The term “therapeutically effective amount” is used to indicate an amount of an active compound, or pharmaceutical agent, that elicits the biological or medicinal response indicated. For example, a therapeutically effective amount of compound can be the amount needed to prevent, alleviate or ameliorate symptoms of disease or prolong the survival of the subject being treated. This response may occur in a tissue, system, animal or human and includes alleviation of the signs or symptoms of the disease being treated. Determination of a therapeutically effective amount is well within the capability of those skilled in the art, in view of the disclosure provided herein. The dose can be tailored to achieve a desired effect, but will depend on such factors as weight, diet, concurrent medication and other factors which those skilled in the medical arts will recognize.

As used herein, the term “coadministration” of pharmacologically active compounds refers to the delivery of two or more separate chemical entities, whether in vitro or in vivo. Coadministration refers to the simultaneous delivery of separate agents; to the simultaneous delivery of a mixture of agents; as well as to the delivery of one agent followed by delivery of a second agent or additional agents. In all cases, agents that are coadministered are intended to work in conjunction with each other.

As used herein, “Cₐ to C_{b}” and “Cₐ-C_{b}” in which “a” and “b” are integers refer to the number of carbon atoms in a saturated or unsaturated fatty acid, fatty alcohol, or a fatty acid portion of a glyceryl ester. The carbon atoms in the saturated or unsaturated fatty acid, fatty alcohol, or a fatty acid portion of a glyceryl ester may be substituted with one or more hydroxyl groups.

As used herein, the term “abarelix” refers to abarelix and pharmaceutically acceptable salts thereof, including acetyl-D-β-naphthylalanyl-D-4-chlorophenylalanyl-D-3-pyridylalanyl-L-seryl-L-N-methyl-tyrosyl-D-asparagyl-L-leucyl-L-N(ε)-isopropyl-lysyl-L-prolyl-D-alanyl-amide. Abarelix can include Plenaxis™.

As used herein, the term “abiraterone” refers to abiraterone and pharmaceutically acceptable salts thereof, including abiraterone acetate. Abiraterone includes (3β)-17-(pyridin-3-yl)androsta-5,16-dien-3-ol. Abiraterone includes Abretone and ZYTIGA®.

As used herein, the term “altraric acid” refers to altraric acid and pharmaceutically acceptable salts thereof, including D-altraric acid and (S)-2-methylpiperazine. Altraric acid includes (2S,3R,4S,5S)-2,3,4,5-tetrahydroxyhexanedioic acid.

As used herein, the term “aminoglutethimide” refers to aminoglutethimide and pharmaceutically acceptable salts thereof, including CYTADREN®, aminoglutethimide, d-Aminoglutethimide L-tartrate, and R-(+)-p-Aminoglutethimide (+)-tartrate salt. Aminoglutethimide includes (RS)-3-(4-aminophenyl)-3-ethyl-piperidine-2,6-dione.

As used herein, the term “ARN-509” refers to ARN-509 and pharmaceutically acceptable salts thereof, including JNJ-56021927 and A52. ARN-509 includes 4-(7-(6-cyano-5-(trifluoromethyl)pyridin-3-yl)-8-oxo-6-thioxo-5,7-diazaspiro[3.4]octan-5-yl)-2-fluoro-N-methylbenzamide).

As used herein, the term “bicalutamide” refers to bicalutamide and pharmaceutically acceptable salts thereof, including BICALOX®, CASODEX®, COSUDEX®, Calutide, and Kalumid. Bicalutamide includes N-[4-cyano-3-(trifluoromethyl)phenyl]-3-[(4-fluorophenyl)sulfonyl]-2-hydroxy-2-methylpropanamide.

As used herein, the term “buserelin” refers to buserelin and pharmaceutically acceptable salts thereof, including buserelin acetate. Buserelin includes Bigonist, SUPRADOPIN®, SURFACT®, Profact, Etilamide, and Tiloryth. Buserelin includes (2S)—N-[(2S)-1-[[(2S)-1-[[(2S)-1-[[(2S)-1-[[(2R)-1-[[(2S)-1-[[(2S)-5-(diaminomethylideneamino)-1-[(2S)-2-(ethylcarbamoyl)pyrrolidin-1-yl]-1-oxopentan-2-yl]amino]-4-methyl-1-oxopentan-2-yl]amino]-3-[(2-methylpropan-2-yl)oxy]-1-oxopropan-2-yl]amino]-3-(4-hydroxyphenyl)-1-oxopropan-2-yl]amino]-3-hydroxy-1-oxopropan-2-yl]amino]-3-(1H-indol-3-yl)-1-oxopropan-2-yl]amino]-3-(1H-imidazol-5-yl)-1-oxopropan-2-yl]-5-oxopyrrolidine-2-carboxamide.

As used herein, the term “cetrorelix” refers to cetrorelix and pharmaceutically acceptable salts thereof, including cetrorelix acetate. Cetrorelix includes acetyl-D-3-(2′-naphtyl)-alanine-D-4-chlorophenylalanine-D-3-(3′-pyridyl)-alanine-L-serine-L-tyrosine-D-citrulline-L-leucine-L-arginine-L-proline-D-alanine-amide.

As used herein, the term “cyproterone acetate” refers to cyproterone acetate and pharmaceutically acceptable salts thereof, including Androcur and CYPROSTAT®. Cyproterone acetate can include 1R,3 aS,3bR,7aR,8aS,8bS,8cS,10aS)-1-acetyl-5-chloro-8b,10a-dimethyl-7-oxo-1,2,3,3a,3b,7,7a,8,8a,8b,8c,9,10,10a-tetradecahydrocyclopenta-[a]cyclopropa-[g]phenanthren-1-yl acetate.

The term “degarelix”, as used herein, refers to degarelix and pharmaceutically acceptable salts thereof, including degarelix acetate. Degarelix includes FIRMAGON® (including FIRMAGON® injection). Degarelix includes D-alaninamide, N-acetyl-3-(2-naphthalenyl)-D-alanyl-4-chloro-D-phenylalanyl-3-(3-pyridinyl)-D-alanyl-L-seryl-4-[[[(4S)-hexahydro-2,6-dioxo-4pyrimidinyl]carbonyl]amino]-L-phenylalanyl-4-[(aminocarbonyl)amino]-D-phenylalanyl-L-leucyl-N6-(1-methylethyl)-L-lysyl-L-prolyl.

As used herein, the term “deslorelin” refers to deslorelin and pharmaceutically acceptable salts thereof, including deslorelin acetate. Deslorelin includes SucroMate™ Equine, Ovuplant, and SUPRELORIN®. Deslorelin includes (2S)—N-[(2S)-1-[[(2S)-1-[[(2S)-1-[[(2S)-1-[[(2R)-1-[[(2S)-1-[[(2S)-5-(diaminomethylideneamino)-1-[(2S)-2-(ethylcarbamoyl)pyrrolidin-1-yl]-1-oxopentan-2-yl]amino]-4-methyl-1-oxopentan-2-yl]amino]-3-(1H-indol-3-yl)-1-oxopropan-2-yl]amino]-3-(4-hydroxyphenyl)-1-oxopropan-2-yl]amino]-3-hydroxy-1-oxopropan-2-yl]amino]-3-(1H-indol-3-yl)-1-oxopropan-2-yl]amino]-3-(1H-imidazol-5-yl)-1-oxopropan-2-yl]-5-oxopyrrolidine-2-carboxamide.

As used herein, the term “diethylstilbestrol” refers to diethylstilbestrol and pharmaceutically acceptable salts thereof, including diethylstilbestrol disodium, diethylstilbestrol diphosphate, and diethylstilbestrol dipropionate. Diethylstilboestrol includes DISTILBENE®, Stilbestrol, and Stilphostrol. Diethylstilboestrol includes 4,4′-(3E)-hex-3-ene-3,4-diyldiphenol.

As used herein, the terms “3,3′-diindolylmethane” and “DIM” refer to 3,3′-diindolylmethane and pharmaceutically acceptable salts thereof, including 5,5′-dichloro-diindolylmethane, dinitro-diindolylmethane, and N,N′-dimethoxy-diindolylmethane. DIM can include 3,3′-methanediylbis(1H-indole), 3-(1H-Indol-3-ylmethyl)-1H-indole, and 3,3′-methylenebis-1H-indole.

As used herein, the term “dutasteride” refers to dutasteride and pharmaceutically acceptable salts thereof, including dutasteride acetate. Dutasteride includes Avodart (including Avodart oral). Dutasteride includes (5α,17β)-N-{2,5-bis(trifluoromethyl)phenyl}-3-oxo-4-azaandrost-1-ene-17-carboxamide.

As used herein, the term “enzalutamide” refers to enzalutamide and pharmaceutically acceptable salts thereof. Enzalutamide includes Xtandi (including Xtandi oral). Enzalutamide includes (4-(3-(4-cyano-3-(trifluoromethyl)phenyl)-5,5-dimethyl-4-oxo-2-thioxoimidazolidin-1-yl)-2-fluoro-N-methylbenzamide).

As used herein, the term “epristeride” refers to epristeride and pharmaceutically acceptable salts thereof. Epristeride includes SKF-105,657 and ONO-9302. Epristeride includes (17-(tert-butylcarbamoyl)androsta-3,5-diene-3-carboxylic acid), 7β-(tert-butylaminocarbonyl)androsta-3,5-diene-3-carboxylic acid, (17β)-17-[[(1,1-dimethylethyl)amino]carbonyl]androsta-3,5-diene-3-carboxylic acid, and (17b)-17-[[(1,1-dimethylethyl)amino]carbonyl]-androsta-3,5-diene-3-carboxylic acid.

As used herein, the term “equol” refers to equol and pharmaceutically acceptable salts thereof, including (R,S) equol 4′-sulfate sodium salt. Equol includes (S)-equol and (R)-equol. Equol includes (3S)-3-(4-Hydroxyphenyl)-7-chromanol, (4′,7-isoflavandiol), 7,4′-dihydroxy-isoflavan, 7-hydroxy-3-(4′-hydroxyphenyl)-chroman, and 3,4-dihydro-3-[4-(sulfooxy)phenyl]-2H-1-benzopyran-7-ol sodium salt.

The term “ethylstilbestrol”, as used herein, refers to ethylstilbestrol and pharmaceutically acceptable salts thereof. Ethylstilboestrol includes BRN 3136095 and alpha-ethyl-4,4′-stilbenediol.

As used herein, the term “finasteride” refers to finasteride and pharmaceutically acceptable salts thereof. Finasteride includes MK-906, Proscar and Propecia. Finasteride includes N-(1,1-dimethylethyl)-3-oxo-(5α,17β)-4-azaandrost-1-ene-17-carboxamide.

As used herein, the term “flutamide” refers to flutamide and pharmaceutically acceptable salts thereof, including hydroxyflutamide and 2-amino-5-nitro-4-(trifluoromethyl)phenol. Flutamide includes Eulexin, Flutamin, Cytomid, Flutamide USP25, Cebatrol, Niftholide, and Niftolid. Flutamide includes 2-methyl-N-[4-nitro-3-(trifluoromethyl)phenyl]-propanamide.

As used herein, the term “fosfestrol” refers to fosfestrol and pharmaceutically acceptable salts thereof, including fosfestrol sodium and fosfestrol tetrasodium. Fosfestrol includes fosfestrol, fosfestrolo, Honvan, and Stilbostatin. Fosfestrol includes [4-[4-(4-phosphonooxyphenyl)hex-3-en-3-yl]phenoxy]phosphonic acid and diethylstilbestrol diphosphate.

As used herein, the term “galeterone” refers to galeterone and pharmaceutically acceptable salts thereof. Galeterone includes Tokai TOK-001 and VN/124-1. Galeterone includes (17-(1H-benzimidazol-1-yl)androsta-5,16-dien-3β-01).

As used herein, the term “ganirelix” refers to ganirelix and pharmaceutically acceptable salts thereof, including ganirelix acetate and ganirelix diacetate. Ganirelix includes Antagon, Cetrotide, Ganirelix, and Orgalutran. Ganirelix includes (2S)-1-[(2S)-2-[[(2S)-2-[[(2R)-2-[[(2R)-2-[[(2S)-2-[[(2R)-2-[[(2R)-2-[[(2R)-2-acetamido-3-naphthalen-2-ylpropanoyl]amino]-3-(4-chlorophenyl)propanoyl]amino]-3-pyridin-3-ylpropanoyl]amino]-3-hydroxypropanoyl]amino]-3-(4-hydroxyphenyl)-propanoyl]amino]-6-[bis(ethylamino)methylideneamino]hexanoyl]-amino]-4-methyl-pentanoyl]amino]-6-[bis(ethylamino)methylideneamino]hexanoyl]-N-[(2R)-1-amino-1-oxopropan-2-yl]pyrrolidine-2-carboxamide.

As used herein, the term “genisterin” refers to genisterin and pharmaceutically acceptable salts thereof. Genisterin includes 5,7-dihydroxy-3-(4-hydroxyphenyl)-1-benzopyran-4-one, and 5,7-dihydroxy-3-(4-hydroxyphenyl)chromen-4-one.

As used herein, the term “18β-glycyrrhetinic acid” refers to 18β-glycyrrhetinic acid and glycyrrhetic acid, and pharmaceutically acceptable salts thereof, including Acetoxolone, Enoxolone, carbenoxolone, and 3β-Hydroxy-11-oxo-18β,20β-olean-12-en-29-oic acid. 18β-Glycyrrhetinic acid can include (2S,4aS,6aS,6bR,8aR,10S,12aS,12bR,14bR)-10-hydroxy-2,4a,6a,6b,9,9,12a-heptamethyl-13-oxo-1,2,3,4,4a,5,6,6a,6b,7,8,8a,9,10,11,12,12a,12b,13,14b-icosahydropicene-2-carboxylic acid.

As used herein, the term “goserelin” refers to goserelin and pharmaceutically acceptable salts thereof, including goserelin acetate. Goserelin includes Zoladex. Goserelin includes N-(21-((1H-indol-3-yl)methyl)-1,1-diamino-12-(tert-butoxymethyl)-6-(2-(2-carbamoylhydrazinecarbonyl)cyclopentanecarbonyl)-15-(4-hydroxybenzyl)-18-(hydroxymethyl)-25-(1H-imidazol-5-yl)-9-isobutyl-8,11,14,17,20,23-hexaoxo-2,7,10,13,16,19,22-heptaazapentacos-1-en-24-yl)-5-oxopyrrolidine-2-carboxamide.

As used herein, the term “gossypol” refers to gossypol and pharmaceutically acceptable salts thereof, including gossypol acetate and acetyl gossypol. Gossypol includes AT-101, ApoG2, B-gossypol, and D-gossypol. Gossypol includes 2,2′-bis-(formyl-1,6,7-trihydroxy-5-isopropyl-3-methylnaphthalene).

As used herein, the term “histrelin” refers to histrelin and pharmaceutically acceptable salts thereof, including histrelin acetate. Histrelin includes Vantas and Supprelin LA. Histrelin includes 5-oxo-L-prolyl-L-histidyl-L-tryptophyl-L-seryl-L-tyrosyl-1-benzyl-D-histidyl-L-leucyl-N5-(diaminomethylene)-L-ornithyl-N-ethyl-L-prolinamide.

As used herein, the term “hormone therapy agent” refers to anti-androgens (including steroidal anti-androgens and non-steroidal anti-androgens), estrogens, luteinizing hormone-releasing hormone (LHRH) agonists, and LHRH antagonists, as well as, hormonal ablation therapy. Some hormone therapy agents are compounds that inhibit the synthesis and/or conversion of testosterone, such as orteronel (“testosterone synthesis inhibitors”); whereas, other hormone therapy agents bind to the androgen receptor and thereby inhibit the binding of testosterone to the androgen receptor, such as Casodex (“androgen receptor inhibitor”). Exemplary hormone therapy agents include, but are not limited to, cyproterone acetate, abiraterone, finasteride, flutamide, nilutamide, bicalutamide, diethylstilbestrol (DES), megestrol acetate, fosfestrol, estamustine phosphate, leuprolide, triptorelin, goserelin, histrelin, buserelin, abarelix, degarelix, orteronel, VT-464, enzalutamide, ARN-509, vinclozolin, galeterone, ketoconazole, L-39, aminoglutethimide, prochloraz, dutasteride, izonsteride, turosteride, epristeride, genisterin, gossypol, equol, 18β-glycyrrhetinic acid, altraric acid, N-butylbenzene-sulfonamide, 3,3′-diindolylmethane, deslorelin, nafarelin, cetrorelix, and ganirelix.

As used herein, the term “izonsteride” refers to izonsteride and pharmaceutically acceptable salts thereof. Izonsteride includes ((4aR,10bR)-8-[(4-ethyl-1,3-benzothiazol-2-yl)sulfanyl]-4,10b-dimethyl-1,4,4a,5,6,10b-hexahydrobenzo[f]quinolin-3(2H)-one).

As used herein, the term “ketoconazole” refers to ketoconazole and pharmaceutically acceptable salts thereof, including ketoconazole oxalate. Ketoconazole includes Nizoral, Extina, Xolegel, and Kuric. Ketoconazole includes (1-[4-(4-{[(2R,4S)-2-(2,4-Dichlorophenyl)-2-(1H-imidazol-1-ylmethyl)-1,3-dioxolan-4-yl]methoxy}phenyl)piperazin-1-yl]ethan-1-one).

The term “L-39”, as used herein, refers to L-39 and pharmaceutically acceptable salts thereof. L-39 includes L-39 cpd. L-39 includes (17-(5′-Isoxazolyl)androsta-4,16-dien-3-one).

As used herein, the term “leuprolide” refers to leuprolide and pharmaceutically acceptable salts thereof, including leuprolide acetate. Leuprolide includes leuprorelin, Lupron (including Lupron injection and Lupron depot), Viadur, Eligard, and Leupromer. Leuprolide includes 5-oxo-L-prolyl-L-histidyl-L-tryptophyl-L-seryl-L-tyrosyl-D-leucyl-L-leucyl-L-arginyl-N-ethyl-Lprolinamide acetate.

As used herein, the term “megestrol acetate” refers to megestrol acetate and pharmaceutically acceptable salts thereof. Megestrol acetate includes Megace and Megace ES. Megestrol acetate includes 17α-(acetyloxy)6-methylpregna-4,6-diene-3,20-dione.

As used herein, the term “N-butylbenzenesulfonamide” refers to N-butylbenzene-sulfonamide and pharmaceutically acceptable salts thereof. N-butylbenzenesulfonamide includes Plasthall and Plastonomoll. N-butylbenzenesulfonamide includes N-n-butylamide, N-butylbenzenesulfonamide, benzenesulfonic acide, benzenesulfonic acid butyl amide, and N-butylbenzenesulfonamide.

As used herein, the term “nilutamide” refers to nilutamide and pharmaceutically acceptable salts thereof. Nilutamide includes Nilandron and Anandron. Nilutamide includes 5,5-dimethyl-3-[4-nitro-3-(trifluoromethyl)phenyl]imidazolidine-2,4-dione.

As used herein, the term “nafarelin” refers to nafarelin and pharmaceutically acceptable salts thereof, including nafarelin acetate. Nafarelin includes Nacenyl, Synarel, Synrelina, Nafarelina, and (D-2-Nal6)-LHRH Nafarelin. Nafarelin includes (2R)—N-[(2R)-5-carbamimidamido-1-[(2S)-2-[(carbamoylmethyl)-carbamoyl]-pyrrolidin-1-yl]-1-oxopentan-2-yl]-2-[(2R)-2-[(2R)-2-[(2R)-3-hydroxy-2-[(2S)-2-[(2S)-3-(1H-imidazol-4-yl)-2-{[(2R)-5-oxopyrrolidin-2-yl]formamido}propanamido]-3-(1H-indol-3-yl)propanamido]propanamido]-3-(4-hydroxyphenyl)propanamido]-3-(naphthalen-2-yl)propanamido]-4-methylpentanamide.

As used herein, the term “orteronel” refers to orteronel and pharmaceutically acceptable salts thereof. Orteronel includes TAK-700. Orteronel includes 6-(7-Hydroxy-6,7-dihydro-5H-pyrrolo[1,2-c]imidazol-7-yl)-N-methyl-naphthalene-2-carboxamide.

As used herein, the term “prochloraz” refers to prochloraz and pharmaceutically acceptable salts thereof, including prochloraz amine, prochloraz copper, prochloraz zinc, and prochloraz manganese salts. Prochloraz includes Pesnatal and JMPR 2001. Prochloraz includes (N-propyl-N-[2-(2,4,6-trichlorophenoxy)-ethyl]imidazole-1-carboxamide) and N-propyl-N-[2-(2,4,6-trichlorophenoxy)ethyl]-1H-imidazole-1-carboxamide.

As used herein, the term “triptorelin” refers to triptorelin and pharmaceutically acceptable salts thereof, including triptorelin acetate and triptorelin pamoate. Triptorelin includes Trelstar, Decapeptyl, Diphereline, Gonapeptyl, and Variopeptyl. Triptorelin includes 5-oxo-D-prolyl-L-histidyl-L-tryptophyl-L-seryl-L-tyrosyl-3-(1H-indol-2-yl)-L-alanylleucyl-L-arginyl-L-prolylglycinamide.

As used herein, the term “turosteride” refers to turosteride and pharmaceutically acceptable salts thereof. Turosteride includes FCE-26073. Turosteride includes ((4aR,4bS,6aS,7S,9aS,9bS,11aR)-1,4a,6a-trimethyl-2-oxo-N-(propan-2-yl)-N-(propan-2-ylcarbamoyl)hexadecahydro-1H-indeno[5,4-f]quinoline-7-carboxamide), and 1-(4-methyl-3-oxo-4-aza-5-alpha-androstane-17-beta-carbonyl)-1,3-diisopropylurea.

As used herein, the term “vinclozolin” refers to vinclozolin and pharmaceutically acceptable salts thereof. Vinclozolin includes Ronilan, Curalan, Vorlan, and Touche. Vinclozolin includes ((RS)-3-(3,5-dichlorophenyl)-5-methyl-5-vinyloxazolidine-2,4-dione).

The term “VT-464”, as used herein, refers to VT-464 and pharmaceutically acceptable salts thereof, including VT-464 racemate and VT-464 R enantiomer. VT-464 refers to the non-steroidal selective CYP17A1 inhibitor developed by Viamet Pharmaceuticals.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. The section below describes some of the pharmaceutical compositions that can be used to treat cancer, or inhibit or delay the growth of cancer cells, especially prostate cancer cells alone or in combination with one or more androgen deprivation therapies (e.g., castration, hormonal castration, hormonal ablation, or hormone therapy).

### II. Pharmaceutical Compositions of Compound I

The present disclosure relates to pharmaceutical compositions containing Compound I. Solubility issues for Compound I make it difficult to produce pharmaceutical compositions of Compound I that are suitable for in vivo use in the clinic. An example of a formulation of Compound I that is difficult to administer in the clinic may include dissolution of Compound I in various organic chemistry solvents or in polyethylene glycol (PEG). Such formulations may be administered to animal subjects by intra-peritoneal injection or oral gavage, however, the methods of administering these formulations are undesirable for human usage in a clinical setting. In particular, it is desirable for a pharmaceutical composition of Compound I that can be administered to patients orally in a clinical setting. Thus, there is a need for new pharmaceutical compositions of Compound I that are acceptable for oral administration to human patients in a clinical setting. It is therefore in some alternatives of this disclosure to provide improved pharmaceutical compositions of Compound I suitable for oral administration to human patients.

The thermodynamic solubility of Compound I was evaluated in aqueous-based and organic solvent media. As disclosed herein, Compound I was found to be mostly insoluble in aqueous media. It was further found that Compound I has good solubility in organic solvents such as alcohols, acetone and other solvents. It was determined that Compound I has good solubility in mixtures of triacylglycerols. In some embodiments, an improved pharmaceutical composition of Compound I may include Compound I and at least one pharmaceutically acceptable carrier. In some embodiments, the at least one pharmaceutically acceptable carrier may be a mixture containing triacylglycerols.

In some embodiments, the mixture containing triacylglycerols may be liquid at room temperature. In some embodiments, the mixture containing triacylglycerols may be liquid at a subjects body temperature. In some embodiments, the mixture containing triacylglycerols may be liquid at a temperature up to 5° C., up to 10° C., up to 15° C., up to 20° C., up to 25° C., up to 30° C., up to 35° C., up to 40° C., up to 45° C., up to 50° C., up to 5° C., up to 60° C., up to 65° C., up to 70° C., up to 75° C., up to 80° C., up to 85° C., up to 90° C., up to 95° C., or more than 95° C.

In some embodiments, the pharmaceutical composition of Compound I that includes Compound I and at least one pharmaceutically acceptable carrier may have a viscosity at 25° C. that ranges from or any number in between 20-250 milliPascal seconds (mPa·s). In some embodiments, the viscosity of the pharmaceutical composition including Compound I and at least one pharmaceutically acceptable carrier at 25° C. ranges from or any number in between 10-15 mPa·s, 12-17 mPa·s, 15-20 mPa·s, 17-22 mPa·s, 20-25 mPa·s, 22-27 mPa·s, 25-30 mPa·s, 27-32 mPa·s, 30-35 mPa·s, 32-37 mPa·s, 35-40 mPa·s, 37-42 mPa·s, 40-45 mPa·s, 42-47 mPa·s, 45-50 mPa·s, 47-52 mPa·s, 50-55 mPa·s, 52-57 mPa·s, 55-60 mPa·s, 67-62 mPa·s, 60-65 mPa·s, 62-67 mPa·s, 65-70 mPa·s, 67-72 mPa·s, 70-80 mPa·s, 75-85 mPa·s, 80-90 mPa·s, 85-95 mPa·s, 90-100 mPa·s, 95-105 mPa·s, 100-110 mPa·s, 105-115 mPa·s, 110-120 mPa·s, 115-125 mPa·s, 120-140 mPa·s, 130-150 mPa·s, 140-160 mPa·s, 150-170 mPa·s, 160-180 mPa·s, 170-190 mPa·s, 180-200 mPa·s, 190-210 mPa·s, 200-220 mPa·s, 210-230 mPa·s, 220-240 mPa·s, 230-250 mPa·s, or 240-260 mPa·s or within a range defined by any two of the aforementioned viscosities. In some embodiments, the viscosity of the pharmaceutical composition including Compound I and at least one pharmaceutically acceptable carrier at 25° C. ranges from or any number in between 200-250 mPa·s, 225-275 mPa·s, 250-300 mPa·s, 300-350 mPa·s, 325-375 mPa·s, 350-400 mPa·s, 400-450 mPa·s, 425-475 mPa·s, 450-500 mPa·s, 500-550 mPa·s, 525-575 mPa·s, 550-600 mPa·s, 600-700 mPa·s, 650-750 mPa·s, 700-800 mPa·s, 750-850 mPa·s, 800-900 mPa·s, 850-950 mPa·s, 900-1000 mPa·s, or 950-1050 mPa·s or within a range defined by any two of the aforementioned viscosities. In some embodiments, the viscosity of the pharmaceutical composition including Compound I and at least one pharmaceutically acceptable carrier at 25° C. is greater than 1000 mPa·s. In some embodiments, the pharmaceutical composition including Compound I and at least one pharmaceutically acceptable carrier at 25° C. is a solid. In some embodiments, the pharmaceutical composition including Compound I and at least one pharmaceutically acceptable carrier at 25° C. is a wax.

In some embodiments, the viscosity of the pharmaceutical composition including Compound I and at least one pharmaceutically acceptable carrier at 37° C. ranges from or any number in between 20-250 milliPascal seconds (mPa·s). In some embodiments, the viscosity of the pharmaceutical composition including Compound I and at least one pharmaceutically acceptable carrier at 37° C. ranges from or any number in between 10-15 mPa·s, 12-17 mPa·s, 15-20 mPa·s, 17-22 mPa·s, 20-25 mPa·s, 22-27 mPa·s, 25-30 mPa·s, 27-32 mPa·s, 30-35 mPa·s, 32-37 mPa·s, 35-40 mPa·s, 37-42 mPa·s, 40-45 mPa·s, 42-47 mPa·s, 45-50 mPa·s, 47-52 mPa·s, 50-55 mPa·s, 52-57 mPa·s, 55-60 mPa·s, 67-62 mPa·s, 60-65 mPa·s, 62-67 mPa·s, 65-70 mPa·s, 67-72 mPa, 70-80 mPa·s, 75-85 mPa·s, 80-90 mPa·s, 85-95 mPa·s, 90-100 mPa·s, 95-105 mPa·s, 100-110 mPa·s, 105-115 mPa·s, 110-120 mPa·s, 115-125 mPa·s, 120-140 mPa·s, 130-150 mPa·s, 140-160 mPa·s, 150-170 mPa·s, 160-180 mPa·s, 170-190 mPa·s, 180-200 mPa·s, 190-210 mPa·s, 200-220 mPa·s, 210-230 mPa·s, 220-240 mPa·s, 230-250 mPa·s, or 240-260 mPa·s or within a range defined by any two of the aforementioned viscosities. In some embodiments, the viscosity of the pharmaceutical composition including Compound I and at least one pharmaceutically acceptable carrier at 37° C. ranges from or any number in between 200-250 mPa·s, 225-275 mPa·s, 250-300 mPa·s, 300-350 mPa·s, 325-375 mPa·s, 350-400 mPa·s, 400-450 mPa·s, 425-475 mPa·s, 450-500 mPa·s, 500-550 mPa·s, 525-575 mPa·s, 550-600 mPa·s, 600-700 mPa·s, 650-750 mPa·s, 700-800 mPa·s, 750-850 mPa·s, 800-900 mPa·s, 850-950 mPa·s, 900-1000 mPa·s, or 950-1050 mPa·s or within a range defined by any two of the aforementioned viscosities. In some embodiments, the viscosity of the pharmaceutical composition including Compound I and at least one pharmaceutically acceptable carrier at 37° C. is greater than 1000 mPa·s. In some embodiments, the pharmaceutical composition including Compound I and at least one pharmaceutically acceptable carrier at 37° C. is a solid. In some embodiments, the pharmaceutical composition including Compound I and at least one pharmaceutically acceptable carrier at 37° C. is a wax.

In some embodiments, the viscosity of the pharmaceutical composition including Compound I and at least one pharmaceutically acceptable carrier is higher than the viscosity of the pharmaceutically acceptable carrier alone. In some embodiments, the viscosity of the pharmaceutical composition including Compound I and at least one pharmaceutically acceptable carrier is lower than the viscosity of the pharmaceutically acceptable carrier alone. In some embodiments, the viscosity of the pharmaceutical composition including Compound I and at least one pharmaceutically acceptable carrier is approximately the same as the viscosity of the pharmaceutically acceptable carrier alone.

In some embodiments, an improved pharmaceutical composition of Compound I may include Compound I, at least one pharmaceutically acceptable carrier, and at least one excipient. In some embodiments, the at least one excipient may be a binder, a disintegrant, a surfactant, or a stabilizer.

In some embodiments, the pharmaceutical composition is formulated as an emulsion. As used herein, emulsion refers to a colloidal dispersion of two immiscible liquids, for example, an oil and water (or other aqueous liquid, e.g., a polar solvent), one of which is part of a continuous phase and the other of which is part of a dispersed phase. The compositions described herein include emulsions, such as oil-in-water nanoemulsions or microemulsions (which may include an oil soluble phase dispersed in an aqueous phase, also called the water phase), in which the oil phase is the dispersed phase and the water phase is the continuous phase. Emulsions may be stabilized by one or more surfactants and/or co-surfactants and/or emulsion stabilizers. Surfactants form an interfacial film between the oil and water phase of the emulsion, providing stability. Nanoemulsions of the provided compositions may contain micelles, containing one or more surfactant surrounding a non-polar active ingredient, which are dispersed in the water phase. Exemplary of the provided emulsions are the provided liquid nanoemulsion concentrates and liquid dilution compositions made by diluting the concentrates, typically in an aqueous medium.

Emulsions may be formed by homogenization using high-speed and/or high-pressure homogenization. In high-speed homogenization, an emulsion may be formed by mixing the surfactant in water in a homogenizer at 500-100,000 rpm, such as 500, 1,000, 2,000, 3,000, 4,000, 5,000, 6,000, 7,000, 8,000, 9,000, 10,000, 15,000, 20,000, 25,000, 30,000, 40,000, 50,000, 60,000, 70,000, 80,000, 90,000, or 100,000 rpm, or an amount within a range defined by any two of the aforementioned values. In high-pressure homogenization, a coarse dispersion of an oil and aqueous phase may be passed through a small inlet orifice at an operating pressure in the range of 500-100,000 psi, such as 500, 1,000, 2,000, 3,000, 4,000, 5,000, 6,000, 7,000, 8,000, 9,000, 10,000, 15,000, 20,000, 25,000, 30,000, 40,000, 50,000, 60,000, 70,000, 80,000, 90,000, or 100,000 psi, or an amount within a range defined by any two of the aforementioned values, where the emulsion mixture is subjected to intense turbulence and hydraulic shear which may produce a fine emulsion with a small droplet size.

Emulsions may also be formed by microfuidization. Microfluidization may use a high pressure positive displacement pump operating at high pressures, such as up to 20,000 psi, which forces the emulsion product through an interaction chamber which consists of a series of microchannels. The emulsion flows through the microchannels on to an impringement area resulting in very fine emulsion droplets. The operating pressure and the number of passes of the coarse emulsion through the interaction chamber of the microfluidizer determine the particle size of the fine emulsion. The higher the operating pressure and the number of passes, the smaller the droplet size of the final emulsion. The resulting emulsion can then be filtered through a 0.2 μm filter to remove any large particles present resulting in a uniform emulsion.

As used herein, an emulsion may be referred to as a nanoemulsion or a microemulstion. In some embodiments, an emulsion may have a diameter (particle size) less than (but not zero) 1000 nm, such as less than 1000 nm, 900 nm, 800 nm, 700 nm, 600 nm, 500 nm, 400 nm, 300 nm, 250 nm, 200 nm, for example, less than (but not zero) 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, or 200 nm. In some embodiments, an emulsion may have a diameter less than (but not zero) 1000 μm, such as less than 1000 μm, 900 μm, 800 μm, 700 μm, 600 μm, 500 μm, 400 μm, 300 μm, 250 μm, 200 μm, for example, less than (but not zero) 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, or 200 μm.

The emulsion diameter may be measured using a dynamic light scattering (DLS) instrument, such as a Zetasizer (Malvern; including Zetasizer 4000, Zetasizer Nano S90, or Zetasizer Nano ZS). For determining the emulsion size, a Z-average diameter fit may be used; this fit can additionally give the polydispersity index (PDI).

In some embodiments, the pharmaceutical formulation is formulated as an emulsion, and includes Compound I and a pharmaceutically acceptable carrier, wherein the carrier forms an emulsion. In some embodiments, Compound I is present in the pharmaceutical formulation in an amount that ranges from or any number in between 1-5 mg/mL, 2-7 mg/mL, 5-10 mg/mL, 7-12 mg/mL, 10-15 mg/mL, 12-17 mg/mL, 15-20 mg/mL, 17-22 mg/mL, 20-25 mg/mL, 22-27 mg/mL, 25-30 mg/mL, 27-32 mg/mL, 30-35 mg/mL, 32-37 mg/mL, 35-40 mg/mL, 37-42 mg/mL, 40-45 mg/mL, 40-50 mg/mL, 45-55 mg/mL, 50-60 mg/mL, 55-65 mg/mL, 60-70 mg/mL, 65-75 mg/mL, 70-80 mg/mL, 75-85 mg/mL, 80-90 mg/mL, 85-95 mg/mL, 90-100 mg/mL, 95-105 mg/mL, 100-110 mg/mL, 105-115 mg/mL, 110-120 mg/mL, 115-125 mg/mL, 120-130 mg/mL, 125-135 mg/mL, 130-140 mg/mL, 135-145 mg/mL, 140-150 mg/mL, 145-155 mg/mL, 150-160 mg/mL, 155-165 mg/mL, 160-170 mg/mL, 165-175 mg/mL, 170-180 mg/mL, 175-185 mg/mL, 180-190 mg/mL, 185-195 mg/mL, 190-200 mg/mL, 195-205 mg/mL, 200-210 mg/mL, 205-215 mg/mL, 210-220 mg/mL, 215-225 mg/mL, 220-230 mg/mL, 225-235 mg/mL, 230-240 mg/mL, 235-245 mg/mL, 240-250 mg/mL, 245-255 mg/mL, 250-300 mg/mL, 275-325 mg/mL, 300-350 mg/mL, 325-375 mg/mL, 350-400 mg/mL, 375-425 mg/mL, 400-450 mg/mL, 425-475 mg/mL, 450-500 mg/mL, 475-525 mg/mL, 500-550 mg/mL, 525-575 mg/mL, or 550-600 mg/mL or within a range defined by any two of the aforementioned amounts. In some embodiments, the concentration of Compound I in the at least one pharmaceutically acceptable carrier ranges from or any number in between 600-650 mg/mL, 625-675 mg/mL, 650-700 mg/mL, 675-725 mg/mL, 700-750 mg/mL, 725-775 mg/mL, 750-800 mg/mL, 775-825 mg/mL, 800-850 mg/mL, 825-875 mg/mL, 850-900 mg/mL, 875-925 mg/mL, 900-950 mg/mL, 925-975 mg/mL, or 950-1000 mg/mL or within a range defined by any two of the aforementioned amounts.

A. Exemplary Pharmaceutically Acceptable Carriers

In some embodiments, the pharmaceutically acceptable carrier may comprise glycerol. In some embodiments, the pharmaceutically acceptable carrier may comprise a mixture of triacylglycerols, monoacylglycerols, diacylglycerols, and free fatty acids. In some embodiments, the pharmaceutically acceptable carrier may comprise a mixture of triacylglycerols. In some embodiments, the pharmaceutically acceptable carrier may comprise a mixture of long chain (C₈-C₅₀) saturated or unsaturated fatty acids, fatty alcohols, or glyceryl esters of one or more fatty acids. In some embodiments, the long chain saturated or unsaturated fatty acids may contain from 10 to 40 carbon atoms (C₁₀-C₄₀). In some embodiments, the long chain saturated or unsaturated fatty acids may contain from 10 to 20 carbon atoms (C₁₀-C₂₀). The mixture of triacylglycerols, monoacylglycerols, diacylglycerols, and free fatty acids can be an oil or wax at room temperature.

Triacylglycerols are triesters of glycerol and three long chain saturated or unsaturated fatty acids; the general chemical formula of a triacylglycerol is shown below:

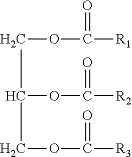

wherein R₁, R₂, and R₃ are saturated or unsaturated C₈-C₅₀ hydrocarbons optionally substituted with one or more hydroxyl groups. In some embodiments, each of R₁, R₂, and R₃ can be the same. In some embodiments, each of R₁, R₂, and R₃ can be different. In some embodiments, R₁ can be the same as R₂, and R₃ can be different than R₁ and R₂. In some embodiments, R₁ can be the same as R₃, and R₂ can be different than R₁ and R₃.

Diacylglycerols are diesters of glycerol and two long chain saturated or unsaturated fatty acids; the general chemical formula of a diacylglycerol is shown below:

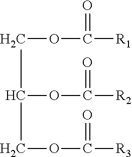

wherein one of R₁, R₂, and R₃ is hydrogen, and the remaining two are saturated or unsaturated C₈-C₅₀ hydrocarbons optionally substituted with one or more hydroxyl groups. In some embodiments, R₁ is hydrogen, and R₂ and R₃ can be the same saturated or unsaturated C₈-C₅₀ hydrocarbon. In some embodiments, R₁ is hydrogen, and R₂ and R₃ can be different saturated or unsaturated C₈-C₅₀ hydrocarbons. In some embodiments, R₂ is hydrogen, and R₁ and R₃ can be the same saturated or unsaturated C₈-C₅₀ hydrocarbon. In some embodiments, R₂ is hydrogen, and R₁ and R₃ can be different saturated or unsaturated C₈-C₅₀ hydrocarbons.

Monoacylglycerols are monoesters of glycerol and one long chain saturated or unsaturated fatty acid; the general chemical formula of a monoacylglycerol is shown below:

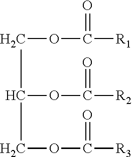

wherein two of R₁, R₂, and R₃ are each hydrogen, and the remaining one is a saturated or unsaturated C₈-C₅₀ hydrocarbon optionally substituted with one or more hydroxyl groups. In some embodiments, R₁ and R₂ are each hydrogen, and R₃ is a saturated or unsaturated C₈-C₅₀ hydrocarbon. In some embodiments, R₁ and R₃ are each hydrogen, and R₂ is a saturated or unsaturated C₈-C₅₀ hydrocarbon.

In some embodiments, each long chain saturated or unsaturated fatty acid is selected from caproic acid, caprylic acid, capric acid, lauric acid, myristic acid, palmitic acid, stearic acid, oleic acid, linolenic acid (including alpha-linolenic acid and gamma-linolenic acid), linoleic acid, arachidic acid, ricinoleic acid, dihydroxystearic acid, behenic acid, ligoceric acid, erucic acid, and/or gondoic acid. In some embodiments, each long chain saturated or unsaturated fatty acid is selected from (Z)-9-octadecenoic acid, oleic acid (8CI), 9-cis-octadecenoic acid, 9Z-octadecenoic acid, B 115, Capryol™ 90 (propylene glycol monocaprylate), Clear FRAC EF, Crodacid O-P, Crossential O 94, D 100, D 100 (fatty acid), Edenor ATiO5, Edenor FTiO5, Emersol 205, Emersol 211, Emersol 213NF, Emersol 214NF, Emersol 233, Emersol 6313NF, Extra Oleic 80R, Extra Oleic 90, Extra Oleic 99, Extra Olein 80, Extra Olein 90, Extra Olein 90R, Extra Olein A 1981, Industrene 105, Lunac O-CA, Lunac O-LL, Lunac O-P, Lunac O-V, Lunac OA, NAA 35, NAA 38, Neo-Fat 92-04, Oleine 7503, Pamolyn 100, Priolene 6204, Priolene 6906, Priolene 6907, Priolene 6928, Priolene 6930, Priolene 6933, Vopcolene 27, Wecoline OO, and/or cis-oleic acid.

In some embodiments, the pharmaceutically acceptable carrier may comprise a mixture of triacylglycerols having long chain saturated or unsaturated fatty acids comprising ricinoleic acid, oleic acid, linoleic acid, palmitic acid, stearic acid, and/or dihydroxystearic acid. In some embodiments, the pharmaceutically acceptable carrier may comprise a mixture of triacylglycerols, where the mixture of triacylglycerols have a fatty acid content comprising 85-95% ricinoleic acid, 2-8% oleic acid, 1-6% linoleic acid, 0.5-3% palmitic acid, 0.5-1% stearic acid, and 0.3-0.7% dihydroxystearic acid. In some embodiments, the pharmaceutically acceptable carrier may comprise a mixture of triacylglycerols, where the mixture of triacylglycerols have a fatty acid content comprising 85-95% ricinoleic acid, 2-8% oleic acid, 1-6% linoleic acid, and 0.5-3% palmitic acid. In some embodiments, the pharmaceutically acceptable carrier may comprise a mixture of triacylglycerols, where the mixture of triacylglycerols have a fatty acid content comprising 85-95% ricinoleic acid, 2-8% oleic acid, and 1-6% linoleic acid. In some embodiments, the pharmaceutically acceptable carrier may comprise a mixture of triacylglycerols, where the mixture of triacylglycerols have a fatty acid content comprising 86-90% ricinoleic acid, 7% oleic acid, 5% linoleic acid, 2% palmitic acid, 1% stearic acid, and 0.7% dihydroxystearic acid. In some embodiments, the pharmaceutically acceptable carrier may comprise a mixture of triacylglycerols, where the mixture of triacylglycerols have a fatty acid content comprising 87% ricinoleic acid, 7% oleic acid, 3% linoleic acid, 2% palmitic acid, 1% stearic acid, and 0.7% dihydroxystearic acid. In some embodiments, the pharmaceutically acceptable carrier may comprise a mixture of triacylglycerols obtained from the seeds of Ricinus communis L., Euphorbiaceae.

In some embodiments, the pharmaceutically acceptable carrier may comprise a mixture of triacylglycerols having long chain saturated or unsaturated fatty acids comprising lauric acid, myristic acid, palmitic acid, oleic acid, caprylic acid, stearic acid, capric acid, caproic acid, linoleic acid, and/or arachidic acid. In some embodiments, the pharmaceutically acceptable carrier may comprise a mixture of triacylglycerols, where the mixture of triacylglycerols have a fatty acid content comprising 44-52% lauric acid, 13-19% myristic acid, 8-11% palmitic acid, 6-10% capric acid, 5-8% oleic acid, 5-9% caprylic acid, 1-3% stearic acid, 0-1% linoleic acid, 0-0.8% caproic acid, and 0-0.5% arachidic acid. In some embodiments, the pharmaceutically acceptable carrier may comprise a mixture of triacylglycerols, where the mixture of triacylglycerols have a fatty acid content comprising 44-52% lauric acid, 13-19% myristic acid, 8-11% palmitic acid, 6-10% capric acid, 5-8% oleic acid, 5-9% caprylic acid, and 1-3% stearic acid. In some embodiments, the pharmaceutically acceptable carrier may comprise a mixture of triacylglycerols, where the mixture of triacylglycerols have a fatty acid content comprising 44-52% lauric acid, 13-19% myristic acid, 8-11% palmitic acid, 6-10% capric acid, 5-8% oleic acid, and 5-9% caprylic acid. In some embodiments, the pharmaceutically acceptable carrier may comprise a mixture of triacylglycerols, where the mixture of triacylglycerols have a fatty acid content comprising 48% lauric acid, 16% myristic acid, 9% palmitic acid, 8% capric acid, 7% oleic acid, and 7% caprylic acid. In some embodiments, the pharmaceutically acceptable carrier may comprise a mixture of triacylglycerols obtained from kernels of Cocos nucifera L., Palmae.

In some embodiments, the pharmaceutically acceptable carrier may comprise a mixture of triacylglycerols having long chain saturated or unsaturated fatty acids comprising linoleic acid, oleic acid, palmitic acid, capric acid, caprylic acid, stearic acid, and/or myristic acid. In some embodiments, the pharmaceutically acceptable carrier may comprise a mixture of triacylglycerols, where the mixture of triacylglycerols have a fatty acid content comprising 34-62% linoleic acid, 19-49% oleic acid, 8-12% palmitic acid, 7% capric acid, 4% caprylic acid, 2-5% stearic acid, and 0.2-1% myristic acid. In some embodiments, the pharmaceutically acceptable carrier may comprise a mixture of triacylglycerols, where the mixture of triacylglycerols have a fatty acid content comprising 34-62% linoleic acid, 19-49% oleic acid, 8-12% palmitic acid, 7% capric acid, 4% caprylic acid, and 2-5% stearic acid. In some embodiments, the pharmaceutically acceptable carrier may comprise a mixture of triacylglycerols, where the mixture of triacylglycerols have a fatty acid content comprising 34-62% linoleic acid, 19-50% oleic acid, 8-19% palmitic acid, 1-4% stearic acid, and 1-2% linolenic acid. In some embodiments, the pharmaceutically acceptable carrier may comprise a mixture of triacylglycerols obtained from the grain of Zea mays L., Gramineae.

In some embodiments, the pharmaceutically acceptable carrier may comprise a mixture of triacylglycerols having long chain saturated or unsaturated fatty acids comprising linoleic acid, oleic acid, palmitic acid, stearic acid, and/or myristic acid. In some embodiments, the pharmaceutically acceptable carrier may comprise a mixture of triacylglycerols, where the mixture of triacylglycerols have a fatty acid content comprising 40-63% linoleic acid, 13-44% oleic acid, 17-29% palmitic acid, 1-4% stearic acid, 0.5-2% myristic acid, and 0.1-2% alpha-linolenic acid. In some embodiments, the pharmaceutically acceptable carrier may comprise a mixture of triacylglycerols, where the mixture of triacylglycerols have a fatty acid content comprising 40-63% linoleic acid, 13-44% oleic acid, and 17-29% palmitic acid. In some embodiments, the pharmaceutically acceptable carrier may comprise a mixture of triacylglycerols, where the mixture of triacylglycerols have a fatty acid content comprising 42% linoleic acid, 35% oleic acid, 20% palmitic acid, 2% stearic acid, and 0.4% myristic acid. In some embodiments, the pharmaceutically acceptable carrier may comprise a mixture of triacylglycerols obtained from the seeds of Gossypium hirsutum L., Malvaceae. In some embodiments, the pharmaceutically acceptable carrier may comprise a mixture of triacylglycerols obtained from the seeds of Gossypium herbaceum L., Malvaceae.

In some embodiments, the pharmaceutically acceptable carrier may comprise a mixture of triacylglycerols having long chain saturated or unsaturated fatty acids comprising linoleic acid, oleic acid, palmitic acid, stearic acid, alpha-linolenic acid, and/or palmitoleic acid. In some embodiments, the pharmaceutically acceptable carrier may comprise a mixture of triacylglycerols, where the mixture of triacylglycerols have a fatty acid content comprising 60-75% linoleic acid, 12-25% oleic acid, 6-9% palmitic acid, 3-6% stearic acid, 0-1.5% alpha-linolenic acid, and 0-1% palmitoleic acid. In some embodiments, the pharmaceutically acceptable carrier may comprise a mixture of triacylglycerols, where the mixture of triacylglycerols have a fatty acid content comprising 60-75% linoleic acid, 12-25% oleic acid, 6-9% palmitic acid, and 3-6% stearic acid. In some embodiments, the pharmaceutically acceptable carrier may comprise a mixture of triacylglycerols, where the mixture of triacylglycerols have a fatty acid content comprising 65% linoleic acid, 17% oleic acid, 8% palmitic acid, and 4% stearic acid. In some embodiments, the pharmaceutically acceptable carrier may comprise a mixture of triacylglycerols, where the mixture of triacylglycerols have a fatty acid content comprising 70% linoleic acid, 16% oleic acid, 7% palmitic acid, and 4% stearic acid. In some embodiments, the pharmaceutically acceptable carrier may comprise a mixture of triacylglycerols obtained from the seeds of Vitis vinifera L., Vitaceae.

In some embodiments, the pharmaceutically acceptable carrier may comprise a mixture of triacylglycerols having long chain saturated or unsaturated fatty acids comprising oleic acid, palmitic acid, linoleic acid, stearic acid, myristic acid, and/or arachidic acid. In some embodiments, the pharmaceutically acceptable carrier may comprise a mixture of triacylglycerols, where the mixture of triacylglycerols have a fatty acid content comprising 65-80% oleic acid, 7-16% palmitic acid, 4-10% linoleic acid, 1-3% stearic acid, 0.1-1% myristic acid, and 0.1-0.3% arachidic acid. In some embodiments, the pharmaceutically acceptable carrier may comprise a mixture of triacylglycerols, where the mixture of triacylglycerols have a fatty acid content comprising 65-80% oleic acid, 7-16% palmitic acid, 4-10% linoleic acid, and 1-3% stearic acid. In some embodiments, the pharmaceutically acceptable carrier may comprise a mixture of triacylglycerols, where the mixture of triacylglycerols have a fatty acid content comprising 55-83% oleic acid, 8-20% palmitic acid, 4-21% linoleic acid, 0.5-5% stearic acid, and 0-1.5% alpha-linolenic acid. In some embodiments, the pharmaceutically acceptable carrier may comprise a mixture of triacylglycerols, where the mixture of triacylglycerols have a fatty acid content comprising 84% oleic acid, 9% palmitic acid, 4% linoleic acid, 2% stearic acid, and 1% arachidic acid. In some embodiments, the pharmaceutically acceptable carrier may comprise a mixture of triacylglycerols obtained from the fruit of Olea europaea L, Oleaceae.

In some embodiments, the pharmaceutically acceptable carrier may comprise a mixture of triacylglycerols having long chain saturated or unsaturated fatty acids comprising oleic acid, palmitic acid, linoleic acid, stearic acid, and/or myristic acid. In some embodiments, the pharmaceutically acceptable carrier may comprise a mixture of triacylglycerols, where the mixture of triacylglycerols have a fatty acid content comprising 38-52% oleic acid, 32-45% palmitic acid, 5-11% linoleic acid, 2-7% stearic acid, and 0.5-2% myristic acid. In some embodiments, the pharmaceutically acceptable carrier may comprise a mixture of triacylglycerols, where the mixture of triacylglycerols have a fatty acid content comprising 38-52% oleic acid, 32-45% palmitic acid, 5-11% linoleic acid, and 2-7% stearic acid. In some embodiments, the pharmaceutically acceptable carrier may comprise a mixture of triacylglycerols, where the mixture of triacylglycerols have a fatty acid content comprising 37% oleic acid, 43% palmitic acid, 9% linoleic acid, 4% stearic acid, and 1% myristic acid. In some embodiments, the pharmaceutically acceptable carrier may comprise a mixture of triacylglycerols obtained from the fruit of Elaeis guineensis, Arecaceae. In some embodiments, the pharmaceutically acceptable carrier may comprise a mixture of triacylglycerols obtained from the fruit of Elaeis oleifera, Arecaceae. In some embodiments, the pharmaceutically acceptable carrier may comprise a mixture of triacylglycerols obtained from the fruit of Attalea maripa, Arecaceae.

In some embodiments, the pharmaceutically acceptable carrier may comprise a mixture of triacylglycerols having long chain saturated or unsaturated fatty acids comprising oleic acid, linoleic acid, palmitic acid, stearic acid, behenic acid, arachidic acid, and/or lignoceric acid. In some embodiments, the pharmaceutically acceptable carrier may comprise a mixture of triacylglycerols, where the mixture of triacylglycerols have a fatty acid content comprising 47-56% oleic acid, 26-33% linoleic acid, and 8-10% palmitic acid. In some embodiments, the pharmaceutically acceptable carrier may comprise a mixture of triacylglycerols, where the mixture of triacylglycerols have a fatty acid content comprising 56% oleic acid, 26% linoleic acid, 8% palmitic acid, 3% stearic acid, 3% behenic acid, 2-3% arachidic acid, and 1% lignoceric acid. In some embodiments, the pharmaceutically acceptable carrier may comprise a mixture of triacylglycerols, where the mixture of triacylglycerols have a fatty acid content comprising 56% oleic acid, 26% linoleic acid, and 8% palmitic acid. In some embodiments, the pharmaceutically acceptable carrier may comprise a mixture of triacylglycerols, where the mixture of triacylglycerols have a fatty acid content comprising 47% oleic acid, 33% linoleic acid, and 10% palmitic acid. In some embodiments, the pharmaceutically acceptable carrier may comprise a mixture of triacylglycerols obtained from the seeds of Arachis hypogaea L., Leguminosae.

In some embodiments, the pharmaceutically acceptable carrier may comprise a mixture of triacylglycerols having long chain saturated or unsaturated fatty acids comprising erucic acid, oleic acid, gondonic acid, linoleic acid, alpha-linolenic acid, palmitic acid, and/or stearic acid. In some embodiments, the pharmaceutically acceptable carrier may comprise a mixture of triacylglycerols, where the mixture of triacylglycerols have a fatty acid content comprising 41% erucic acid, 17% oleic acid, 15% gondonic acid, 13% linoleic acid, 9% alpha-linolenic acid, 4% palmitic acid, and 1.5% stearic acid. In some embodiments, the pharmaceutically acceptable carrier may comprise a mixture of triacylglycerols, where the mixture of triacylglycerols have a fatty acid content comprising 41% erucic acid, 17% oleic acid, 15% gondonic acid, 13% linoleic acid, 9% alpha-linolenic acid, and 4% palmitic acid. In some embodiments, the pharmaceutically acceptable carrier may comprise a mixture of triacylglycerols obtained from the seeds of Brassica napus L., Brassicaceae.

In some embodiments, the pharmaceutically acceptable carrier may comprise a mixture of triacylglycerols having long chain saturated or unsaturated fatty acids comprising oleic acid, linoleic acid, alpha-linolenic acid, palmitic acid, gondonic acid, and/or stearic acid. In some embodiments, the pharmaceutically acceptable carrier may comprise a mixture of triacylglycerols, where the mixture of triacylglycerols have a fatty acid content comprising 61-63% oleic acid, 20-21% linoleic acid, 9-11% alpha-linolenic acid, 4% palmitic acid, 2% gondonic acid, 2% stearic acid, and less than 2% erucic acid. In some embodiments, the pharmaceutically acceptable carrier may comprise a mixture of triacylglycerols, where the mixture of triacylglycerols have a fatty acid content comprising 63% oleic acid, 20% linoleic acid, 9% alpha-linolenic acid, 4% palmitic acid, 2% gondonic acid, and 2% stearic acid. In some embodiments, the pharmaceutically acceptable carrier may comprise a mixture of triacylglycerols, where the mixture of triacylglycerols have a fatty acid content comprising 61% oleic acid, 21% linoleic acid, 9% alpha-linolenic acid, 4% palmitic acid, 2% gondonic acid, and 2% stearic acid. In some embodiments, the pharmaceutically acceptable carrier may comprise a mixture of triacylglycerols obtained from the seeds of Brassica napus L., Brassicaceae. In some embodiments, the pharmaceutically acceptable carrier may comprise a mixture of triacylglycerols obtained from the seeds of Brassica rapa L., Brassicaceae. In some embodiments, the pharmaceutically acceptable carrier may comprise a mixture of triacylglycerols obtained from the seeds of Brassica juncea L., Brassicaceae.

In some embodiments, the pharmaceutically acceptable carrier may comprise a mixture of triacylglycerols having long chain saturated or unsaturated fatty acids comprising linoleic acid, oleic acid, palmitic acid, and/or stearic acid. In some embodiments, the pharmaceutically acceptable carrier may comprise a mixture of triacylglycerols, where the mixture of triacylglycerols have a fatty acid content comprising 73-79% linoleic acid, 13-21% oleic acid, 3-6% palmitic acid, and 1-4% stearic acid. In some embodiments, the pharmaceutically acceptable carrier may comprise a mixture of triacylglycerols, where the mixture of triacylglycerols have a fatty acid content comprising 72-84% linoleic acid, 7-42% oleic acid, 2-10% palmitic acid, and 1-10% stearic acid. In some embodiments, the pharmaceutically acceptable carrier may comprise a mixture of triacylglycerols, where the mixture of triacylglycerols have a fatty acid content comprising 77-79% linoleic acid, 13% oleic acid, 6% palmitic acid, and 3% stearic acid. In some embodiments, the pharmaceutically acceptable carrier may comprise a mixture of triacylglycerols obtained from the seeds of Carthamus tinctorius L., Compositae.

In some embodiments, the pharmaceutically acceptable carrier may comprise a mixture of triacylglycerols having long chain saturated or unsaturated fatty acids comprising oleic acid, linoleic acid, palmitic acid, stearic acid, and/or arachidic acid. In some embodiments, the pharmaceutically acceptable carrier may comprise a mixture of triacylglycerols having long chain saturated or unsaturated fatty acids comprising oleic acid, linoleic acid, palmitic acid, and stearic acid. In some embodiments, the pharmaceutically acceptable carrier may comprise a mixture of triacylglycerols, where the mixture of triacylglycerols have a fatty acid content comprising 40-50% oleic acid, 35-45% linoleic acid, 7-9% palmitic acid, 4-5% stearic acid, and 0.4-1% arachidic acid. In some embodiments, the pharmaceutically acceptable carrier may comprise a mixture of triacylglycerols, where the mixture of triacylglycerols have a fatty acid content comprising 40-50% oleic acid, 35-45% linoleic acid, 7-9% palmitic acid, and 4-5% stearic acid. In some embodiments, the pharmaceutically acceptable carrier may comprise a mixture of triacylglycerols, where the mixture of triacylglycerols have a fatty acid content comprising 39% oleic acid, 41% linoleic acid, 8% palmitic acid, and 5% stearic acid. In some embodiments, the pharmaceutically acceptable carrier may comprise a mixture of triacylglycerols obtained from the seeds of Sesamum indicum L., Pedaliaceae.

In some embodiments, the pharmaceutically acceptable carrier may comprise a mixture of triacylglycerols having long chain saturated or unsaturated fatty acids comprising linoleic acid, oleic acid, palmitic acid, linolenic acid, and/or stearic acid. In some embodiments, the pharmaceutically acceptable carrier may comprise a mixture of triacylglycerols, where the mixture of triacylglycerols have a fatty acid content comprising 43-56% linoleic acid, 22-34% oleic acid, 7-11% palmitic acid, 5-11% linolenic acid, and 2-6% stearic acid. In some embodiments, the pharmaceutically acceptable carrier may comprise a mixture of triacylglycerols, where the mixture of triacylglycerols have a fatty acid content comprising 44-62% linoleic acid, 19-30% oleic acid, 7-14% palmitic acid, 4-11% linolenic acid, and 1-6% stearic acid. In some embodiments, the pharmaceutically acceptable carrier may comprise a mixture of triacylglycerols, where the mixture of triacylglycerols have a fatty acid content comprising 49% linoleic acid, 26% oleic acid, 10% palmitic acid, 11% linolenic acid, and 4% stearic acid. In some embodiments, the pharmaceutically acceptable carrier may comprise a mixture of triacylglycerols obtained from the seeds of Glycine max L., Fabaceae.

In some embodiments, the pharmaceutically acceptable carrier may comprise a mixture of triacylglycerols having long chain saturated or unsaturated fatty acids comprising linoleic acid, oleic acid, palmitic acid, stearic acid, arachidic acid, and/or behenic acid. In some embodiments, the pharmaceutically acceptable carrier may comprise a mixture of triacylglycerols, where the mixture of triacylglycerols have a fatty acid content comprising 44-75% linoleic acid, 14-35% oleic acid, 3-6% palmitic acid, 1-3% stearic acid, 0.6-4% arachidic acid, and 1% behenic acid. In some embodiments, the pharmaceutically acceptable carrier may comprise a mixture of triacylglycerols, where the mixture of triacylglycerols have a fatty acid content comprising 63-73% linoleic acid, 14-24% oleic acid, 3-10% palmitic acid, 2-8% stearic acid, and 0-3% linolenic acid. In some embodiments, the pharmaceutically acceptable carrier may comprise a mixture of triacylglycerols, where the mixture of triacylglycerols have a fatty acid content comprising 66% linoleic acid, 21% oleic acid, 6% palmitic acid, 1% stearic acid, 4% arachidic acid, and 1% behenic acid. In some embodiments, the pharmaceutically acceptable carrier may comprise a mixture of triacylglycerols obtained from the seeds of Helianthus annuus L., Compositae.

In some embodiments, the pharmaceutically acceptable carrier may comprise an oil selected from castor oil, coconut oil, corn oil, cottonseed oil, grapeseed oil, olive oil, palm oil, peanut oil, rapeseed oil, canola oil, safflower oil, sesame oil, soybean oil, or sunflower oil, or combinations thereof. In some embodiments, the pharmaceutically acceptable carrier may be sesame oil. In some embodiments, the pharmaceutically acceptable carrier is not sesame oil.

In some embodiments, the pharmaceutically acceptable carrier may comprise Labrafac™ lipophile WL1349, which is a triacylglycerol of caprylic acid and capric acid. In some embodiments, the pharmaceutically acceptable carrier may comprise Akoline MCM, Akomed E, Arlamol M 812, C8-10 glycerides, Capmul MCM, Capric/caprylic triglyceride, Capric/caprylic triglycerides, Caprylic/capric triglyceride, Caprylic/capric triglycerides, Captex 300, Captex 300 Low C6, Captex 300EP, Captex 335, Captex 355, Coconad MT, Coconard MT, Crodamol GTCC, Crodamol PC-DAB 10(S), Delios 325, Delios SK, Delios V, Delios V MCT oil, Delios VK koscher, Dermol M 5, Estasan 3575, Estasan GT 8-60, Estasan GT 8-65, Estol 1527, Estol 3601, Estol 3603, Ethox 2156, Kolliphor RH40, Labrafac CCTG, Labrafac LIPO WL 1349, Labrafac Lipophile, Labrafac Lipophile WL 1349, Labrafac WL 1349, Labrasol, Lexol GT 865, Liponate GC, Lumulse CC 33K, Miglyol 810, Miglyol 810N, Miglyol 812, Miglyol 812N, Miglyol 812S, Myritol 312, Myritol 314, Myritol 318, Myritol 325, Neobee M 5, Neobee O, Neobee Oil M 5, Neoderm TCC, Nikkol Triester F 810, O.D.O., Panacet 810, Panacete, Panacete 810, Radia 7104, Rofetan GTCC, Rylo TG 50, Sefol 880, Stepan 108, Stepan-Mild GCC, Sun Crystal, Surfac MCTG, Tegosoft CT, Triester F 810, Triglycerides C8-10, or Velsan CCT, or combinations thereof. In some embodiments, the pharmaceutically acceptable carrier may comprise Peceol™, which is a monoacylglycerol of oleic acid.

In some embodiments, the pharmaceutically acceptable carrier may comprise a mixture of triacylglycerols and further comprise one or more phospholipids. In some embodiments, the phospholipid may have the general formula shown below:

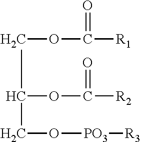

wherein R₁ and R₂ are saturated or unsaturated C₈-C₅₀ hydrocarbons optionally substituted with one or more hydroxyl groups; and R₃ is hydrogen or a hydrophilic head group. In some embodiments, the hydrophilic head group is ethanolamine, choline, serine, inositol, or glycerol, In some embodiments, R₁ and R₂ can be the same saturated or unsaturated C₈-C₅₀ hydrocarbon. In some embodiments, R₁ and R₂ can be different saturated or unsaturated C₈-C₅₀ hydrocarbons. In some embodiments, the one or more phospholipids can be a mixture of one or more of phosphatidic acid, phosphatidylcholine, phosphatidylethanolamine, phosphatidylglycerol, phosphatidylinositol, phosphatidylserine, or lysophosphatidylcholine, or combinations thereof. In some embodiments, the one or more phospholipids can be selected from 1,2-dierucoyl-sn-glycero-3-phosphate, 1,2-dierucoyl-sn-glycero-3-phosphocholine, 1,2-dierucoyl-sn-glycero-3-phosphoethanolamine, 1,2-dilauroyl-sn-glycero-3-phosphate, 1,2-dilauroyl-sn-glycero-3-phosphocholine, 1,2-dilauroyl-sn-glycero-3-phosphoethanolamine, 1,2-dilauroyl-sn-glycero-3-phosphoserine, 1,2-dilinoleoyl-sn-glycero-3-phosphocholine, 1,2-dimyristoyl-sn-glycero-3-phosphate, 1,2-dimyristoyl-sn-glycero-3-phosphocholine, 1,2-dimyristoyl-sn-glycero-3-phosphoethanolamine, 1,2-dimyristoyl-sn-glycero-3-phosphoserine, 1,2-dioleoyl-sn-glycero-3-phosphate, 1,2-dioleoyl-sn-glycero-3-phosphocholine, 1,2-dioleoyl-sn-glycero-3-phosphoethanolamine, 1,2-dioleoyl-sn-glycero-3-phospho serine, 1,2-dipalmitoyl-sn-glycero-3-phosphate, 1,2-dipalmitoyl-sn-glycero-3-phosphocholine, 1,2-dipalmitoyl-sn-glycero-3-phosphoethanolamine, 1,2-dipalmitoyl-sn-glycero-3-phosphoserine, 1,2-distearoyl-sn-glycero-3-phosphate, 1,2-distearoyl-sn-glycero-3-phosphocholine, 1,2-distearoyl-sn-glycero-3-phosphoethanolamine, 1,2-distearoyl-sn-glycero-3-phosphoserine, 1-myristoyl-2-palmitoyl-sn-glycero 3-phosphocholine, 1-myristoyl-2-stearoyl-sn-glycero-3-phosphocholine, 1-myristoyl-sn-glycero-3-phosphocholine, 1-palmitoyl-2-myristoyl-sn-glycero-3-phosphocholine, 1-palmitoyl-2-oleoyl-sn-glycero-3-phosphocholine, 1-palmitoyl-2-oleoyl-sn-glycero-3-phosphoethanolamine, 1-palmitoyl-2-stearoyl-sn-glycero-3-phosphocholine, 1-palmitoyl-sn-glycero-3-phosphocholine, 1-stearoyl-2-myristoyl-sn-glycero-3-phosphocholine, 1-stearoyl-2-oleoyl-sn-glycero-3-phosphocholine, 1-stearoyl-2-palmitoyl-sn-glycero-3-phosphocholine, or 1-stearoyl-sn-glycero-3-phosphocholine, or any combination thereof.

In some embodiments, the pharmaceutically acceptable carrier may comprise a mixture of triacylglycerols and further comprise dimethyl sulfoxide. Dimethyl sulfoxide may be used as a pharmaceutically acceptable carrier to facilitate the uptake of Compound I into cells or tissues of a subject. Dimethyl sulfoxide may be used as a pharmaceutically acceptable carrier to facilitate absorption of Compound I in the gastrointestinal tract of a subject.

In some embodiments, the pharmaceutically acceptable carrier may comprise propylene glycol. In some embodiments, the pharmaceutically acceptable carrier may comprise esters of propylene glycol. In some embodiments, the ester of propylene glycol can be propylene glycol monocaproate, propylene glycol monocaprylate, propylene glycol monodecanoate, propylene glycol monolaurate, propylene glycol monomyristate, propylene glycol monopalmitate, propyleneglycol monostearate, propylene glycol monooleate, propylene glycol monolinolenate, propylene glycol dicaproate, propylene glycol dicaprylate, propylene glycol didecanoate, propylene glycol dilaurate, propylene glycol dimyristate, propylene glycol dipalmitate, propyleneglycol distearate, propylene glycol dioleate, or propylene glycol dilinolenate, or any combination thereof. In some embodiments, the pharmaceutically acceptable carrier may comprise propylene carbonate. In some embodiments, the pharmaceutically acceptable carrier may comprise Capryol™ 90, which is propylene glycol monocaprylate.

In some embodiments, the pharmaceutically acceptable carrier may comprise esters of polyethylene glycol. In some embodiments, the ester of polyethylene glycol can be PEG-8, PEG-10, PEG-25, PEG-55, PEG 75, PEG 120, or PEG 660 monocaproate or dicaproate; PEG-8, PEG-10, PEG-25, PEG-55, PEG 75, PEG 120, or PEG 660 monocaprylate or dicaprylate; PEG-8, PEG-10, PEG-25, PEG-55, PEG 75, PEG 120, or PEG 660 monodecanoate or didecanoate; PEG-8, PEG-10, PEG-25, PEG-55, PEG 75, PEG 120, or PEG 660 monolaurate or dilaurate; PEG-8, PEG-10, PEG-25, PEG-55, PEG 75, PEG 120, or PEG 660 monomyristate or dimyristate; PEG-8, PEG-10, PEG-25, PEG-55, PEG 75, PEG 120, or PEG 660 monopalmitate or dipalmitate; PEG-8, PEG-10, PEG-25, PEG-55, PEG 75, PEG 120, or PEG 660 monostearate or distearate; PEG-8, PEG-10, PEG-25, PEG-55, PEG 75, PEG 120, or PEG 660 monooleate or dioleate; PEG-8, PEG-10, PEG-25, PEG-55, PEG 75, PEG 120, or PEG 660 monolinolenate or dilinolenate; or any combination thereof. In some embodiments, the pharmaceutically acceptable carrier may comprise diethylene glycol monoethyl ether.

In some embodiments, the pharmaceutically acceptable carrier may comprise a pegylated glyceride. Exemplary pegylated glycerides include GELUCIRE® 44/14 (lauroyl macrogol-32 glycerides) and GELUCIRE® 50/13 (stearoyl macrogol-32 glycerides).

In some embodiments, the pharmaceutically acceptable carrier may comprise a fatty acid ester having the general formula R₁—C(═O)—O—R₂, where R₁ and R₂ are each saturated or unsaturated C8-C50 hydrocarbons optionally substituted with one or more hydroxyl groups. In some embodiments, each of R₁ and R₂ can be the same. In some embodiments, each of R₁ and R₂ can be different. In some embodiments, the fatty acid ester can be methyl caproate, ethyl caproate, propyl caproate, isopropyl caproate, butyl caproate, sec-butyl caproate, tert-butyl caproate, pentyl caproate, hexyl caproate, heptyl caproate, octyl caproate, nonyl caproate, or decyl caproate, or any combination thereof. In some embodiments, the fatty acid ester can be methyl caprylate, ethyl caprylate, propyl caprylate, isopropyl caprylate, butyl caprylate, sec-butyl caprylate, tert-butyl caprylate, pentyl caprylate, hexyl caprylate, heptyl caprylate, octyl caprylate, nonyl caprylate, or decyl caprylate, or any combination thereof. In some embodiments, the fatty acid ester can be methyl decanoate, ethyl decanoate, propyl decanoate, isopropyl decanoate, butyl decanoate, sec-butyl decanoate, tert-butyl decanoate, pentyl decanoate, hexyl decanoate, heptyl decanoate, octyl decanoate, nonyl decanoate, or decyl decanoate, or any combination thereof. In some embodiments, the fatty acid ester can be methyl laurate, ethyl laurate, propyl laurate, isopropyl laurate, butyl laurate, sec-butyl laurate, tert-butyl laurate, pentyl laurate, hexyl laurate, heptyl laurate, octyl laurate, nonyl laurate, or decyl laurate, or any combination thereof. In some embodiments, the fatty acid ester can be methyl myristate, ethyl myristate, propyl myristate, isopropyl myristate, butyl myristate, sec-butyl myristate, tert-butyl myristate, pentyl myristate, hexyl myristate, heptyl myristate, octyl myristate, nonyl myristate, or decyl myristate, or any combination thereof. In some embodiments, the fatty acid ester can be methyl palmitate, ethyl palmitate, propyl palmitate, isopropyl palmitate, butyl palmitate, sec-butyl palmitate, tert-butyl palmitate, pentyl palmitate, hexyl palmitate, heptyl palmitate, octyl palmitate, nonyl palmitate, or decyl palmitate, or any combination thereof. In some embodiments, the fatty acid ester can be methyl stearate, ethyl stearate, propyl stearate, isopropyl stearate, butyl stearate, sec-butyl stearate, tert-butyl stearate, pentyl stearate, hexyl stearate, heptyl stearate, octyl stearate, nonyl stearate, or decyl stearate, or any combination thereof. In some embodiments, the fatty acid ester can be methyl oleate, ethyl oleate, propyl oleate, isopropyl oleate, butyl oleate, sec-butyl oleate, tert-butyl oleate, pentyl oleate, hexyl oleate, heptyl oleate, octyl oleate, nonyl oleate, or decyl oleate, or any combination thereof. In some embodiments, the fatty acid ester can be methyl linolenate, ethyl linolenate, propyl linolenate, isopropyl linolenate, butyl linolenate, sec-butyl linolenate, tert-butyl linolenate, pentyl linolenate, hexyl linolenate, heptyl linolenate, octyl linolenate, nonyl linolenate, or decyl linolenate, or any combination thereof.

In some embodiments, the pharmaceutically acceptable carrier may comprise a sorbitan ester. In some embodiments, the sorbitan ester can be sorbitan monolaurate, sorbitan monopalmitate, sorbitan monostearate, sorbitan monooleate, sorbitan sesquioleate, sorbitan trioleate, or sorbitan isostearate, or any combination thereof. In some embodiments, the sorbitan ester can be Span 20, Span 40, Span 60, Span 80, Span 83, Span 85, Span 120, or any combination thereof.

In some embodiments, the pharmaceutically acceptable carrier may comprise a polysorbate. In some embodiments, the polysorbate can be polyoxyethylene (20) sorbitan monolaurate, polyoxyethylene (20) sorbitan monopalmitate, polyoxyethylene (20) sorbitan monostearate, polyoxyethylene (20) sorbitan monooleate, polyoxyethylene (4) sorbitan monolaurate, polyoxyethylene (4) sorbitan monopalmitate, or polyoxyethylene (4) sorbitan monostearate, polyoxyethylene (4) sorbitan monooleate, polysorbate 80 or any combination thereof. In some embodiments, the polysorbate can be Tween 20, Tween 21, Tween 40, Tween 60, Tween 61, Tween 65, or Tween 80, or any combination thereof.

In some embodiments, the pharmaceutically acceptable carrier facilitates the oral administration of Compound I in a pharmaceutical composition for use in the clinic. In some embodiments, the pharmaceutically acceptable carrier facilitates the oral administration of Compound I to patients that are not hospitalized. In some embodiments, the pharmaceutically acceptable carrier facilitates the oral administration of Compound I to patients in a painless and convenient dosage form. In some embodiments, the pharmaceutically acceptable carrier described herein is unexpectedly superior to formulations of Compound I that use organic chemistry solvents or PEG.

In some embodiments, the amount of pharmaceutically acceptable carrier(s) may vary from or any number in between 25% to 85% by weight of the total pharmaceutical composition. In some embodiments, the amount of pharmaceutically acceptable carrier(s) ranges from or any number in between 25%-30%, 27%-32%, 30%-35%, 32%-37%, 35%-40%, 37%-42%, 40%-45%, 42%-47%, 45%-50%, 47%-52%, 50%-55%, 52%-57%, 55%-60%, 67%-72%, 70%-75%, 72%-77%, 75%-80%, 77%-82%, or 80%-85% by weight of the total pharmaceutical composition or within a range defined by any two of the aforementioned percentages. In some embodiments, the amount of pharmaceutically acceptable carrier(s) may be at least 15%, at least 20%, at least 25%, at least 30%, at least 35%, at least 40%, at least 45%, at least 50%, at least 55%, at least 60%, at least 65%, at least 70%, at least 75%, at least 80%, or at least 85% of the weight of the total pharmaceutical composition or within a range defined by any two of the aforementioned percentages.

In some embodiments, Compound I is dissolved or suspended in the at least one pharmaceutically acceptable carrier that comprises a mixture of triacylglycerols, monoacylglycerols, diacylglycerols, and free fatty acids. In some embodiments, the concentration of Compound I dissolved or suspended in the at least one pharmaceutically acceptable carrier that comprises a mixture of triacylglycerols, monoacylglycerols, diacylglycerols, and free fatty acids may vary from or any number in between 5-100 mg of Compound I per mL of pharmaceutically acceptable carrier. In some embodiments, the concentration of Compound I in the at least one pharmaceutically acceptable carrier ranges from or any number in between 1-5 mg/mL, 2-7 mg/mL, 5-10 mg/mL, 7-12 mg/mL, 10-15 mg/mL, 12-17 mg/mL, 15-20 mg/mL, 17-22 mg/mL, 20-25 mg/mL, 22-27 mg/mL, 25-30 mg/mL, 27-32 mg/mL, 30-35 mg/mL, 32-37 mg/mL, 35-40 mg/mL, 37-42 mg/mL, 40-45 mg/mL, 40-50 mg/mL, 45-55 mg/mL, 50-60 mg/mL, 55-65 mg/mL, 60-70 mg/mL, 65-75 mg/mL, 70-80 mg/mL, 75-85 mg/mL, 80-90 mg/mL, 85-95 mg/mL, 90-100 mg/mL, 95-105 mg/mL, 100-110 mg/mL, 105-115 mg/mL, 110-120 mg/mL, 115-125 mg/mL, 120-130 mg/mL, 125-135 mg/mL, 130-140 mg/mL, 135-145 mg/mL, 140-150 mg/mL, 145-155 mg/mL, 150-160 mg/mL, 155-165 mg/mL, 160-170 mg/mL, 165-175 mg/mL, 170-180 mg/mL, 175-185 mg/mL, 180-190 mg/mL, 185-195 mg/mL, 190-200 mg/mL, 195-205 mg/mL, 200-210 mg/mL, 205-215 mg/mL, 210-220 mg/mL, 215-225 mg/mL, 220-230 mg/mL, 225-235 mg/mL, 230-240 mg/mL, 235-245 mg/mL, 240-250 mg/mL, 245-255 mg/mL, 250-300 mg/mL, 275-325 mg/mL, 300-350 mg/mL, 325-375 mg/mL, 350-400 mg/mL, 375-425 mg/mL, 400-450 mg/mL, 425-475 mg/mL, 450-500 mg/mL, 475-525 mg/mL, 500-550 mg/mL, 525-575 mg/mL, or 550-600 mg/mL or within a range defined by any two of the aforementioned amounts. In some embodiments, the concentration of Compound I in the at least one pharmaceutically acceptable carrier ranges from or any number in between 600-650 mg/mL, 625-675 mg/mL, 650-700 mg/mL, 675-725 mg/mL, 700-750 mg/mL, 725-775 mg/mL, 750-800 mg/mL, 775-825 mg/mL, 800-850 mg/mL, 825-875 mg/mL, 850-900 mg/mL, 875-925 mg/mL, 900-950 mg/mL, 925-975 mg/mL, or 950-1000 mg/mL or within a range defined by any two of the aforementioned amounts.

In some embodiments, the concentration of Compound I in the at least one pharmaceutically acceptable carrier is at least 5 mg/mL, at least 10 mg/mL, at least 15 mg/mL, at least 20 mg/mL, at least 25 mg/mL, at least 30 mg/mL, at least 40 mg/mL, at least 50 mg/mL, at least 60 mg/mL, at least 70 mg/mL, at least 75 mg/mL, at least 80 mg/mL, at least 85 mg/mL, at least 90 mg/mL, at least 95 mg/mL, at least 100 mg/mL, at least 105 mg/mL, least 110 mg/mL, at least 115 mg/mL, least 120 mg/mL, at least 125 mg/mL, least 130 mg/mL, at least 135 mg/mL, least 140 mg/mL, at least 145 mg/mL, at least 150 mg/mL, at least 175 mg/mL, at least 200 mg/mL, at least 225 mg/mL, at least 250 mg/mL, at least 300 mg/mL, at least 350 mg/mL, at least 400 mg/mL, at least 450 mg/mL, at least 500 mg/mL, or at least 550 mg/mL or within a range defined by any two of the aforementioned amounts. In some embodiments, the concentration of Compound I in the at least one pharmaceutically acceptable carrier is at least 600 mg/mL, at least 650 mg/mL, at least 700 mg/mL, at least 750 mg/mL, at least 800 mg/mL, at least 850 mg/mL, at least 900 mg/mL, at least 950 mg/mL, or at least 1000 mg/mL or within a range defined by any two of the aforementioned amounts. In some embodiments, the concentration of Compound I in the at least one pharmaceutically acceptable carrier is greater than 250 mg/mL. In some embodiments, the concentration of Compound I in the at least one pharmaceutically acceptable carrier is greater than 500 mg/mL. In some embodiments, the concentration of Compound I in the at least one pharmaceutically acceptable carrier is greater than 750 mg/mL. In some embodiments, the concentration of Compound I in the at least one pharmaceutically acceptable carrier is greater than 1000 mg/mL.

B. Excipients

One embodiment disclosed herein includes a pharmaceutical composition comprising Compound I, at least one pharmaceutically acceptable carrier, and at least one pharmaceutically acceptable excipient. The at least one pharmaceutically acceptable excipient can be selected from a sugar, a starch, a cellulose preparation, silicon dioxide aerosol, gelatin, calcium phosphate dibasic, sodium lauryl sulfate, magnesium stearate, sodium stearyl fumarate, talc, polyethylene glycol, or polyvinylpyrrolidone, and any combination thereof. In one embodiment, the at least one pharmaceutically acceptable excipient can be selected from a pregelatinized starch, partially pregelatinized starch, microcrystalline cellulose, silicified microcrystalline cellulose, a lactose-cellulose blend, methyl cellulose, or silicon dioxide aerosol, or any combination thereof. In one embodiment, the at least one pharmaceutically acceptable excipient can be selected from microcrystalline cellulose, lactose, sucrose, starch powder, maize starch or derivatives thereof, cellulose esters of alkanoic acids, cellulose alkyl esters, stearic acid, magnesium oxide, sodium and calcium salts of phosphoric and sulfuric acids, acacia gum, sodium alginate, or polyvinyl alcohol, or any combination thereof. In one embodiment, the at least one pharmaceutically acceptable excipient can be selected from dextrose, mannitol, lactose monohydrate, lecithin, albumin, sodium glutamate, cysteine hydrochloride, croscarmellose sodium, sodium starch glycolate, hydroxypropyl cellulose, poloxamer, sodium lauryl sulfate, or colloidal silicon dioxide, or any combination thereof. Poloxamers include, for example, poloxamer 101, 105, 108, 122, 123, 124, 181, 182, 183, 184, 185, 188, 212, 215, 217, 231, 234, 235, 237, 238, 282, 284, 288, 331, 333, 334, 335, 338, 401, 402, 403, 407, poloxamer 105 benzoate, or poloxamer 182 dibenzoate 407 or any combination thereof. In one embodiment, the at least one pharmaceutically acceptable excipient can be microcrystalline cellulose. In one embodiment, the at least one pharmaceutically acceptable excipient can be an aluminometasilicate, such as sodium aluminometasilicate, magnesium aluminometasilicate, calcium aluminometasilicate, potassium aluminometasilicate, or lithium aluminometasilicate.

The amount of the pharmaceutically acceptable excipient may vary from or any number in between 1% to 75% by weight of the total pharmaceutical composition. In some embodiments, the amount of excipient ranges from or any number in between 1-5%, 2-7%, 5-10%, 7-12%, 10-15%, 12-17%, 15%-20%, 17%-22%, 20%-25%, 22%-27%, 25%-30%, 27%-32%, 30%-35%, 32%-37%, 35%-40%, 37%-42%, 40%-45%, 40-50%, 45-55%, 50-60%, 55-65%, 60-70%, or 65-75% by weight of the total pharmaceutical composition or within a range defined by any two of the aforementioned amounts. In some embodiments, the amount of excipient is at least 5%, at least 10%, at least 15%, at least 20%, at least 25%, at least 30%, at least 40%, at least 50%, at least 60%, at least 70%, or at least 75% of the weight of the total pharmaceutical composition or within a range defined by any two of the aforementioned amounts. In some embodiments, the amount of excipient is less than 5% of the weight of the total pharmaceutical composition but not zero.

The amounts of excipient can be determined by the dosage of Compound I and the dosage form size of the total pharmaceutical composition. In some embodiments disclosed herein, the dosage form size of the total pharmaceutical composition is 175 mg. In some embodiments disclosed herein the dosage form size of the total pharmaceutical composition is 350 mg. In some embodiments disclosed herein the dosage form size of the total pharmaceutical composition is 700 mg. One skilled in the art will realize that a range of dosage form sizes of the total pharmaceutical composition can be made and are encompassed by this disclosure. The preferred dosage form size range of the total pharmaceutical composition is from 50 mg to 1500 mg, more typically from 100 mg to 1000 mg, more typically from 175 mg to 700 mg, with the preferred typical dosage form size of the total pharmaceutical composition being 175 mg, 350 mg, or 700 mg or within a range defined by any two of the aforementioned amounts.

In some embodiments, the at least one pharmaceutically acceptable excipient can be selected from binders, disintegrants, surfactants, or stabilizers. Any one or more of the excipients (including binders, disintegrants, surfactants, or stabilizers) can be appropriate in the pharmaceutical composition containing Compound I and at least one pharmaceutically acceptable carrier in accordance with the disclosure herein, provided that Compound I is not inactivated by the formulation and the formulation is physiologically compatible and tolerable with oral administration to a subject.

1. Binders

In some embodiments, the at least one pharmaceutically acceptable excipient can be selected from at least one or more binders. The at least one or more binders can be used, for example, to impart cohesive qualities to a pharmaceutical formulation containing Compound I, and thus permit the resulting dosage form to remain intact during formulation of capsules, tablets, film coated tablets, caplets, gel caps, pill pellets, or beads, suitable for oral administration to a subject. In some embodiments, the one or more binders are selected from microcrystalline cellulose, gelatin, sugars (including, for example, sucrose, glucose, dextrose and maltodextrin), polyethylene glycol, waxes, natural and synthetic gums, polyvinylpyrrolidone, pregelatinized starch, povidone, cellulosic polymers (including, for example, hydroxypropyl cellulose (HPC), hydroxypropyl methylcellulose (HPMC), methyl cellulose, hydroxyethyl cellulose), or hydroxypropyl cellulose (HPC), or any combination thereof.

2. Disintegrants

In some embodiments, the at least one pharmaceutically acceptable excipient can be selected from at least one or more disintegrants. The at least one or more disintegrants can be used, for example, to facilitate disintegration of a pharmaceutical composition after oral administration. In some embodiments, the at least one or more disintegrants are selected from starches, clays, celluloses, algins, gums, or crosslinked polymers. In some embodiments, the one or more disintegrants are selected from crosslinked polyvinylpyrrolidone (PVP-XL), sodium starch glycolate, alginic acid, methacrylic acid DYB, microcrystalline cellulose, crospovidone, polacriline potassium, sodium starch glycolate, starch, pregelatinized starch, or croscarmellose sodium.

3. Surfactants and Co-Surfactants

As used herein, “surfactant” refers to synthetic and naturally occurring amphiphilic molecules that have hydrophobic portion(s) and hydrophilic portion(s). Due to their amphiphilic (amphipathic) nature, surfactants and co-surfactants typically can reduce the surface tension between two immiscible liquids, for example, the oil and water phases in an emulsion, stabilizing the emulsion. Different surfactants can be characterized based on their relative hydrophobicity and/or hydrophilicity. For example, relatively lipophilic surfactants are more soluble in fats, oils and waxes, while relatively hydrophilic surfactants are more soluble in aqueous compositions, for example, water. Relatively amphiphilic surfactants are soluble in oil and water based liquids.

In some embodiments, the at least one pharmaceutically acceptable excipient can be selected from at least one or more surfactants. The at least one or more surfactants can be used, for example, as a wetting agent. The at least one or more surfactants can be used, for example, to improve the permeation and bioavailability of Compound I. In some embodiments, the at least one or more surfactants are selected from anionic surfactants, non-ionic surfactants, or zwitterionic surfactants or any mixture thereof. In some embodiments, the one or more surfactants are selected from poly(oxyethylene) sorbitan fatty acid ester, poly(oxyethylene) stearate, poly(oxyethylene) alkyl ether, polyglycolated glyceride, poly(oxyethylene) castor oil, sorbitan fatty acid ester, poloxamer, fatty acid salt, bile salt, alkyl sulfate, lecithin, mixed micelle of bile salt and lecithin, glucose ester vitamin E TPGS (D-α-tocopheryl polyethylene glycol 1000 succinate), Labrasol™ (caprylocaproyl polyoxyl-8 glycerides), Kolliphor™ RH40 (polyoxyl castor oil), or sodium lauryl sulfate, or combinations thereof. In some embodiments, the surfactant is a nonionic surfactant/co-surfactant mixture, for example Labrasol™ and Kolliphor® RH40 in a ratio of 1:1.

4. Stabilizers

In some embodiments, the at least one pharmaceutically acceptable excipient can be selected from one or more stabilizers. In some embodiments, the at least one or more stabilizers are selected from alkanizing agents, chelating agents, photoprotectants, or antioxidants.

In some embodiments, the alkanizing agent is selected from alkali metal salt additives or an alkaline earth metal salt additive. Alkali metal salt additives suitable for use in some embodiments can be, for example, sodium carbonate, sodium hydroxide, sodium silicate, disodium hydrogen orthophosphate, sodium aluminate and other suitable alkali metal salts. Alkaline earth metal salt additives can include, for example, calcium carbonate, calcium hydroxide, magnesium carbonate, magnesium hydroxide, magnesium silicate, magnesium aluminate, or aluminum magnesium hydroxide.

In some embodiments, the chelating agent is selected from disodium EDTA, edetic acid, or citric acid, or any combination thereof.

In some embodiments, a photoprotectant can be used, for example, to protect the pharmaceutical composition from the chemical or physical effects of light. In some embodiments, the photoprotectant is selected from titanium oxide, ferric oxide, or zinc oxide or any combination thereof.

In some embodiments, the antioxidant is selected from butylated hydroxyanisole (BHA), sodium ascorbate, butylated hydroxytoluene (BHT), sodium sulfite, propyl gallate, tocopherol, citric acid, malic acid, or ascorbic acid, or any mixtures thereof.

### III. Dosage Forms of Pharmaceutical Compositions of Compound I

Compound I may be formulated as a pharmaceutical composition suitable for oral administration to a subject. In some embodiments, the pharmaceutical composition is formulated for oral ingestion by a subject as a tablet, pill, capsule, granule, dragee, liquid, gel, syrup, slurry, spray, or suspension. In some embodiments, the composition is in the form of a tablet, a film coated tablet, a gel cap, a caplet, a pellet, or a bead. In some embodiments, the pharmaceutical composition is in the form of a capsule having a dissolvable enclosure for carrying Compound I and one or more pharmaceutically acceptable carriers and/or excipients. In one embodiment, capsule is made of gelatin. In some embodiments, the pharmaceutical composition is in the form of a soft gel capsule.

Pharmaceutical compositions for oral administration may be obtained by combining Compound I with one or more pharmaceutically acceptable carriers and/or excipients, optionally grinding the resulting mixture, and processing the mixture, to obtain tablets, pills, capsules, granules, dragees, a liquid, a gel, a syrup, a slurry, a spray, or a suspension. The pharmaceutical compositions of Compound I may be manufactured by mixing, dissolving, granulating, dragee-making, levigating, emulsifying, encapsulating, entrapping or tabletting processes.

In some embodiments, the pharmaceutical composition may include a coating, for example, a film coating. Where film coatings are involved, coating preparations can include, for example, a film-forming polymer, or a plasticizer. Non-limiting examples of film-forming polymers suitable for use in the embodiments described herein include hydroxypropyl methylcellulose, hydroxypropyl cellulose, methylcellulose, polyvinyl pyrrolidine, or starches. Non-limiting examples of plasticizers suitable for use in the embodiments described herein include polyethylene glycol, tributyl citrate, dibutyl sebecate, or acetylated monoglyceride. Dyestuffs or pigments may be added to the pharmaceutical composition or to coatings for the pharmaceutical composition for identification or to characterize different combinations of active compound doses. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added for identification or to characterize different dosage amounts of Compound I. Non-limiting examples of dyestuffs and pigments suitable for use in the embodiments described herein include iron oxides of various colors, lake dyes of many colors, or titanium dioxide.

In some embodiments, Compound I may be formulated as a pharmaceutical composition suitable for administration to a subject rectally, transmucosally, topically, via intestinal administration, parenteral delivery (including intramuscular, subcutaneous, intravenous, and intramedullary injections), intrathecally, via direct intraventricular, intraperitoneal, intranasal, or intraocular injection.

The pharmaceutical composition may be presented in a pack or dispenser device, which may contain one or more unit dosage forms containing Compound I. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. The pharmaceutical composition of Compound I may be placed in an appropriate container, and labeled for treatment of an indicated condition, such as for treatment of cancer.

In some embodiments, the relative amounts of Compound I, the pharmaceutically acceptable carrier(s), and excipient(s) by weight that can comprise a pharmaceutical composition of Compound I are shown in Table A.

TABLE A

| | Amount of | |
|---|---|---|
| | pharmaceutically | |
| Amount of Compound I | acceptable carrier(s) | Amount of excipient(s) |
| 1-5% | 95-99% | 0% |
| 1-5% | 90-98% | 1-5% |
| 1-5% | 85-94% | 5-10% |
| 1-5% | 80-89% | 10-15% |
| 1-5% | 75-84% | 15-20% |
| 1-5% | 70-79% | 20-25% |
| 1-5% | 65-74% | 25-30% |
| 1-5% | 60-69% | 30-35% |
| 1-5% | 55-64% | 35-40% |
| 1-5% | 50-59% | 40-45% |
| 1-5% | 45-54% | 45-50% |
| 1-5% | 40-49% | 50-55% |
| 1-5% | 35-44% | 55-60% |
| 1-5% | 30-39% | 60-65% |
| 1-5% | 25-34% | 65-70% |
| 1-5% | 20-29% | 70-75% |
| 1-5% | 15-24% | 75-80% |
| 1-5% | 10-19% | 80-85% |
| 1-5% | 5-14% | 85-90% |
| 1-5% | 1-9% | 90-94% |
| 5-10% | 90-95% | 0% |
| 5-10% | 85-94% | 1-5% |
| 5-10% | 80-90% | 5-10% |
| 5-10% | 75-85% | 10-15% |
| 5-10% | 70-80% | 15-20% |
| 5-10% | 65-75% | 20-25% |
| 5-10% | 60-70% | 25-30% |
| 5-10% | 55-65% | 30-35% |
| 5-10% | 50-60% | 35-40% |
| 5-10% | 45-55% | 40-45% |
| 5-10% | 40-50% | 45-50% |
| 5-10% | 35-45% | 50-55% |
| 5-10% | 30-40% | 55-60% |
| 5-10% | 25-35% | 60-65% |
| 5-10% | 20-30% | 65-70% |
| 5-10% | 15-25% | 70-75% |
| 5-10% | 10-20% | 75-80% |
| 5-10% | 5-15% | 80-85% |
| 5-10% | 1-10% | 85-89% |
| 10-15% | 85-90% | 0% |
| 10-15% | 80-89% | 1-5% |
| 10-15% | 75-85% | 5-10% |
| 10-15% | 70-80% | 10-15% |
| 10-15% | 65-75% | 15-20% |
| 10-15% | 60-70% | 20-25% |
| 10-15% | 55-65% | 25-30% |
| 10-15% | 50-60% | 30-35% |
| 10-15% | 45-55% | 35-40% |
| 10-15% | 40-50% | 40-45% |
| 10-15% | 35-45% | 45-50% |
| 10-15% | 30-40% | 50-55% |
| 10-15% | 25-35% | 55-60% |
| 10-15% | 20-30% | 60-65% |
| 10-15% | 15-25% | 65-70% |
| 10-15% | 10-20% | 70-75% |
| 10-15% | 5-15% | 75-80% |
| 10-15% | 1-10% | 80-84% |
| 15-20% | 80-85% | 0% |
| 15-20% | 75-84% | 1-5% |
| 15-20% | 70-80% | 5-10% |
| 15-20% | 65-75% | 10-15% |
| 15-20% | 60-70% | 15-20% |
| 15-20% | 55-65% | 20-25% |
| 15-20% | 50-60% | 25-30% |
| 15-20% | 45-55% | 30-35% |
| 15-20% | 40-50% | 35-40% |
| 15-20% | 35-45% | 40-45% |
| 15-20% | 30-40% | 45-50% |
| 15-20% | 25-35% | 50-55% |
| 15-20% | 20-30% | 55-60% |
| 15-20% | 15-25% | 60-65% |
| 15-20% | 10-20% | 65-70% |
| 15-20% | 5-15% | 70-75% |
| 15-20% | 1-10% | 75-79% |
| 20-25% | 75-80% | 0% |
| 20-25% | 70-79% | 1-5% |
| 20-25% | 65-75% | 5-10% |
| 20-25% | 60-70% | 10-15% |
| 20-25% | 55-65% | 15-20% |
| 20-25% | 50-60% | 20-25% |
| 20-25% | 45-55% | 25-30% |
| 20-25% | 40-50% | 30-35% |
| 20-25% | 35-45% | 35-40% |
| 20-25% | 30-40% | 40-45% |
| 20-25% | 25-35% | 45-50% |
| 20-25% | 20-30% | 50-55% |
| 20-25% | 15-25% | 55-60% |
| 20-25% | 10-20% | 60-65% |
| 20-25% | 5-15% | 65-70% |
| 20-25% | 1-10% | 70-74% |
| 25-30% | 70-75% | 0% |
| 25-30% | 65-74% | 1-5% |
| 25-30% | 60-70% | 5-10% |
| 25-30% | 55-65% | 10-15% |
| 25-30% | 50-60% | 15-20% |
| 25-30% | 45-55% | 20-25% |
| 25-30% | 40-50% | 25-30% |
| 25-30% | 35-45% | 30-35% |
| 25-30% | 30-40% | 35-40% |
| 25-30% | 25-35% | 40-45% |
| 25-30% | 20-30% | 45-50% |
| 25-30% | 15-25% | 50-55% |
| 25-30% | 10-20% | 55-60% |
| 25-30% | 5-15% | 60-65% |
| 25-30% | 1-10% | 65-69% |
| 30-35% | 65-70% | 0% |
| 30-35% | 60-69% | 1-5% |
| 30-35% | 55-65% | 5-10% |
| 30-35% | 50-60% | 10-15% |
| 30-35% | 45-55% | 15-20% |
| 30-35% | 40-50% | 20-25% |
| 30-35% | 35-45% | 25-30% |
| 30-35% | 30-40% | 30-35% |
| 30-35% | 25-35% | 35-40% |
| 30-35% | 20-30% | 40-45% |
| 30-35% | 15-25% | 45-50% |
| 30-35% | 10-20% | 50-55% |
| 30-35% | 5-15% | 55-60% |
| 30-35% | 1-10% | 60-64% |
| 35-40% | 60-65% | 0% |
| 35-40% | 55-64% | 1-5% |
| 35-40% | 50-60% | 5-10% |
| 35-40% | 45-55% | 10-15% |
| 35-40% | 40-50% | 15-20% |
| 35-40% | 35-45% | 20-25% |
| 35-40% | 30-40% | 25-30% |
| 35-40% | 25-35% | 30-35% |
| 35-40% | 20-30% | 35-40% |
| 35-40% | 15-25% | 40-45% |
| 35-40% | 10-20% | 45-50% |
| 35-40% | 5-15% | 50-55% |
| 35-40% | 1-10% | 55-59% |
| 40-45% | 55-60% | 0% |
| 40-45% | 50-59% | 1-5% |
| 40-45% | 45-55% | 5-10% |
| 40-45% | 40-50% | 10-15% |
| 40-45% | 35-45% | 15-20% |
| 40-45% | 30-40% | 20-25% |
| 40-45% | 25-35% | 25-30% |
| 40-45% | 20-30% | 30-35% |
| 40-45% | 15-25% | 35-40% |
| 40-45% | 10-20% | 40-45% |
| 40-45% | 5-15% | 45-50% |
| 40-45% | 1-10% | 50-54% |
| 45-50% | 50-55% | 0% |
| 45-50% | 45-54% | 1-5% |
| 45-50% | 40-50% | 5-10% |
| 45-50% | 35-45% | 10-15% |
| 45-50% | 30-40% | 15-20% |
| 45-50% | 25-35% | 20-25% |
| 45-50% | 20-30% | 25-30% |
| 45-50% | 15-25% | 30-35% |
| 45-50% | 10-20% | 35-40% |
| 45-50% | 5-15% | 40-45% |
| 45-50% | 1-10% | 45-49% |
| 50-55% | 45-50% | 0% |
| 50-55% | 40-49% | 1-5% |
| 50-55% | 35-45% | 5-10% |
| 50-55% | 30-40% | 10-15% |
| 50-55% | 25-35% | 15-20% |
| 50-55% | 20-30% | 20-25% |
| 50-55% | 15-25% | 25-30% |
| 50-55% | 10-20% | 30-35% |
| 50-55% | 5-15% | 35-40% |
| 50-55% | 1-10% | 40-44% |
| 55-60% | 40-45% | 0% |
| 55-60% | 35-44% | 1-5% |
| 55-60% | 30-40% | 5-10% |
| 55-60% | 25-35% | 10-15% |
| 55-60% | 20-30% | 15-20% |
| 55-60% | 15-25% | 20-25% |
| 55-60% | 10-20% | 25-30% |
| 55-60% | 5-15% | 30-35% |
| 55-60% | 1-10% | 35-39% |
| 60-65% | 35-40% | 0% |
| 60-65% | 30-39% | 1-5% |
| 60-65% | 25-35% | 5-10% |
| 60-65% | 20-30% | 10-15% |
| 60-65% | 15-25% | 15-20% |
| 60-65% | 10-20% | 20-25% |
| 60-65% | 5-15% | 25-30% |
| 60-65% | 1-10% | 30-34% |
| 65-70% | 30-39% | 0% |
| 65-70% | 25-35% | 1-5% |
| 65-70% | 20-30% | 5-10% |
| 65-70% | 15-25% | 10-15% |
| 65-70% | 10-20% | 15-20% |
| 65-70% | 5-15% | 20-25% |
| 65-70% | 1-10% | 25-29% |

### IV. Improved Stability of Pharmaceutical Compositions of Compound I

In addition to the therapeutic properties of the pharmaceutical compositions described herein, it was surprisingly discovered that the pharmaceutical compositions of Compound I result in stable compositions having a long shelf life. Accordingly, some embodiments described herein include stable pharmaceutical formulations of Compound I.

In some embodiments, the pharmaceutical compositions show good stability under various storage conditions over time. In some embodiments, under various storage conditions the pharmaceutical compositions of Compound I can be stable for at least 1 week, 2 weeks, 3 weeks, 1 month, 2 months, 3 months, 6 months, 9 months, 12 months, 15 months, 18 months, 24 months, 36 months, or 48 months or within a range defined by any two of the aforementioned times. In some embodiments, under various storage conditions the pharmaceutical compositions of Compound I can be stable for more than 48 months. For example, under storage conditions of 25° C. and 60% relative humidity, the pharmaceutical compositions of Compound I can be stable for at least, 1 week, 2 weeks, 3 weeks, 1 month, 2 months, 3 months, 6 months, 9 months, 12 months, 15 months, 18 months, 24 months, 36 months, or 48 months or within a range defined by any two of the aforementioned times. Under storage conditions of 25° C. and 60% relative humidity, the pharmaceutical compositions of Compound I can be stable for more than 48 months. In another example, under storage conditions of 40° C. and 75% relative humidity, the pharmaceutical compositions of Compound I can be stable for at least 1 week, 2 weeks, 3 weeks, 1 month, 2 months, 3 months, 6 months, 9 months, 12 months, 15 months, 18 months, 24 months, 36 months, or 48 months or within a range defined by any two of the aforementioned times. Under storage conditions of 40° C. and 75% relative humidity, the pharmaceutical compositions of Compound I can be stable for more than 48 months. The stability of pharmaceutical compositions of Compound I provided herein is demonstrated, for example, in representative Examples 3 and 4, and Tables 4-10.

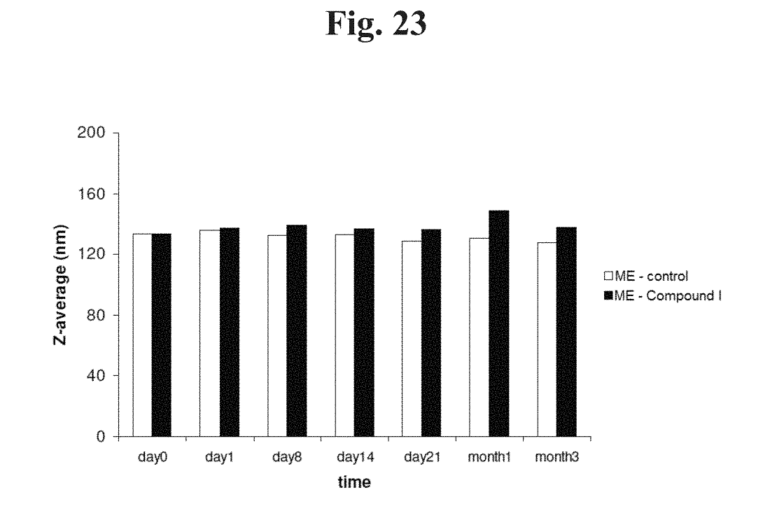

Some embodiments further relate to improved stability in emulsion compositions that include Compound I. In some embodiments, the compositions are stable for a period of 1 day to 5 years, for example, for at least 1 day, 1 week, 2 weeks, 3 weeks, 1 month, 2 months, 3 months, 6 months, 9 months, 12 months, 15 months, 18 months, 24 months, 36 months, or 48 months, or 60 months or within a range defined by any two of the aforementioned times. For example, under storage conditions of 25° C., the emulsion compositions of Compound I can be stable for at least 1 day, 1 week, 2 weeks, 3 weeks, 1 month, 2 months, 3 months, 6 months, 9 months, 12 months, 15 months, 18 months, 24 months, 36 months, 48 months, or 60 months or within a range defined by any two of the aforementioned times. Under storage conditions of 25° C., the emulsion compositions of Compound I can be stable for more than 60 months. The stability of the emulsion compositions including Compound I provided herein is demonstrate, for example, in representative Example 13 and in FIG. 23.

### V. Therapeutic Indications

Some alternatives disclosed herein relate to pharmaceutical compositions of Compound I and methods of using such compositions with and without a hormone therapy agent, as described herein, to inhibit, delay, treat, or prevent prostate cancer cell growth or prostate cancer in a subject in need thereof.

A. Prostate Cancer

Prostate cancer is a leading cause of cancer-related death in the United States and the rest of the world. Advanced prostate cancer is resistant to hormone therapy, radiation and conventional chemotherapy. Although the 5-year survival rate is close to 100% for local disease, it drops to 30% for advanced cancer.

In the initial stages, prostate tumor growth is androgen dependent. Androgens are used by prostate cancer cells for both proliferation as well as regulation, and are vital for maintaining the growth and survival of the cancer cell. The main androgen that circulates is testosterone, which is mainly produced in the testes. Extragonadal sources of androgen synthesis do, however, exist and may play a role in the development of castration-resistant forms of prostate cancer. Generally, androgen dependent prostate cancer therapy focuses on minimizing testicular synthesis of androgens with luteinizing hormone releasing hormone (“LHRH”) agonists or antagonists. Some therapies also focus on modulating the androgen receptor itself, or its downstream signaling pathway.

Androgen dependent prostate cancer eventually progresses into castration-resistant prostate cancer (“CRPC”). Although these patients are “androgen insensitive,” researchers have discovered that androgen-responsive genes are still expressed, implying that the androgen-receptor signaling pathway may still be an important target in CRPC patients.

In 1986, surgeons developed a technique (using da Vinci Prostatectomy) that allowed the removal of the prostate while minimizing nerve damage, thereby decreasing adverse side effects. Unfortunately, should a patient's prostate-specific antigen (PSA) level remain above zero after radical prostatectomy is performed, this indicates that the prostate cancer has spread outside the capsule, i.e., disseminated, and to date, there is no curable treatment for this. Current hormonal and chemotherapy treatment regimens for such disseminated androgen dependent prostate cancers are palliative. Subsequently, even if there have been advances in the treatment of prostate cancer, finding new strategies for treatment of disseminated disease remains a crucial challenge. The section below provides more details on the use of pharmaceutical compositions of Compound I to inhibit, ameliorate, or delay the growth of cancer cells, in particular prostate cancer cells.

Most patients treated with existing hormone therapies eventually relapse with the emergence of castration-resistant prostate cancer (CRPC). Studies have shown that CRPC still relies on androgens for proliferation and that the androgen receptor (AR) is often highly expressed and transcriptionally active in CRPC cells. Steroids from the adrenal glands contribute to androgen receptor activity and some CRPC cells over-express enzymes that mediate androgen synthesis from adrenal steroids, and/or synthesize androgens de novo from cholesterol. Androgen receptor activity driven by residual androgens indeed participates in CRPC.

Prostate cancer can result in damage to the prostatic capsule. Prostate tumor cells can increase in size and number, which can increase the tension of the prostatic capsule. Eventually, the pressure against the prostatic capsule can cause the capsule to rupture. A broken, damaged, or ruptured prostate capsule may result in dissemination of the disease where prostate cancer spreads outside of the capsule. Some alternatives disclosed herein relate to pharmaceutical compositions of Compound I and methods of using such compositions with and without a hormone therapy agent, as described herein, to inhibit, delay, treat, or prevent damage or rupture of the prostatic capsule.

B. Pharmaceutical Compositions of Compound I as Anticancer Agents

Pharmaceutical compositions of Compound I have significant anti-cancer properties for oral administration to subjects in need of anti-cancer treatment. Without wishing to be bound by theory, it is contemplated that the primary mechanism of cytotoxic action of pharmaceutical compositions of Compound I is due to redox-cycling and electrophilic arylation. Compound I may be reduced by electron transfer from flavoprotein to a semiquinone radical, which can, in turn, reduce oxygen to superoxide. The resulting superoxide may consequently be converted into hydrogen peroxide, hydroxyl radicals, and/or peroxynitrite, all of which are highly reactive oxygen species (ROS) with potent cytotoxic and tumoricidial effects.

While still not wishing to be bound by theory, an additional antitumor mechanism of pharmaceutical compositions of Compound I may involve direct arylation of intracellular thiols leading to depletion of glutathione (GSH). Depletion of GSH may ultimately result in alkylation of cellular macromolecules and in their inactivation. Moreover, pharmaceutical compositions of Compound I may inhibit expression of multiple molecular targets, including protein kinase Cq (PKCq), phosphatidylinositol 3-kinase (PI3K), AKT, activation of transcription factors activator protein-1 (AP-1), nuclear factor-κB (NF-κB), and signal transducer and activator of transcription 3 (Stat3) in prostate carcinoma cells. Such activities may contribute to the tumoricidial effects of pharmaceutical compositions of Compound I.

Moreover, while still not wishing to be bound by theory, an additional antitumor mechanism for pharmaceutical compositions of Compound I may involve inhibition of microtubule polymerization and binding to tubulin. Because one of the defining characteristics of cancer cells is a significantly increased rate of cell cycle entry and/or mitosis, cancer cells are more vulnerable to agents that affect microtubule polymerization than normal cells. Compound I may recognize the colchicine binding site of tubulin and inhibit in vitro tubulin polymerization.

Pharmaceutical compositions of Compound I may result in slower growth of androgen independent prostate cancer, and that the mechanism behind the slower growth may be due to apoptosis of prostate tumor cells. Pharmaceutical compositions of Compound I may induce cell cycle entry, mitosis, and/or apoptosis of androgen-dependent cancer cells.

It is contemplated that pharmaceutical compositions of Compound I have anti-cancer activity and that this anti-cancer activity, especially with respect to prostate cancer, may be significantly and unexpectedly improved (e.g., synergy may be obtained) when the pharmaceutical compositions of Compound I are provided to a subject orally in conjunction with a blockade of testosterone/androgen/DHT (e.g., castration or a hormone treatment therapy, such as hormonal ablation). For example, it is believed that the administration of a pharmaceutical composition of Compound I to a subject in need thereof will effectively inhibit the growth of prostate cancer cells and thereby reduce the incidence of fatal prostate cancer. The combination of a pharmaceutical composition of Compound I with an antioxidant, such as ascorbic acid, alpha lipoic acid, n-acetyl cysteine (NAC), lycopene, tocopherol, or tocotrienol, or others may also be beneficial. The combination of a pharmaceutical composition of Compound I and mitomycin C may also be beneficial in treating subjects with advanced solid tumors, advanced lung cancer, and advanced gastrointestinal cancer. By administering a combination of a pharmaceutical composition of Compound I and an antioxidant or plurality of antioxidants, such as vitamin C, to subjects having prostate cancer, it is contemplated that a reduction in tumor cell numbers and PSA (prostate cancer specific antigen) will be obtained.

Alternatively or in addition, it is contemplated that pharmaceutical compositions of Compound I have anti-cancer activity and that this anti-cancer activity, especially with respect to prostate cancer, may be significantly improved (e.g., synergy may be obtained) when the compositions are provided in conjunction with certain hormonal therapy agents, described in more detail below. It is believed that Compound I alters the androgen receptor pathway. Accordingly, it is preferred that pharmaceutical compositions of Compound I are provided in combination or in co-administration with a testosterone synthesis inhibitor that do not alter the androgen receptor pathway (e.g., a testosterone synthesis inhibitor that does not bind to the androgen receptor, such as orteronel or VT-464).

It is contemplated herein that a significantly improved inhibition of prostate cancer cell growth may be obtained when castration, hormonal castration, hormonal ablation, or hormone therapy are provided during the time a patient receives the combination of antioxidant (e.g., ascorbic acid) with a pharmaceutical composition of Compound I. Provided herein is an improved method for treating a subject suffering from prostate cancer with a pharmaceutical composition of Compound I and androgen ablation therapy to subjects with PSA values above zero after radical prostatectomy, i.e., when they have androgen-dependent disseminated disease. Today there is no cure for this and patients currently receive only palliative treatment, including hormone therapy alone.

It is contemplated that pharmaceutical compositions of Compound I are highly oxidative and induce oxidative stress in cells. Accordingly, such compositions may be used to inhibit or ameliorate prostate cancer cell growth and that a significantly improved inhibition or amelioration of prostate cancer cell growth may be obtained when castration, hormonal castration, hormonal ablation, or hormone therapy are provided before, during, and/or after the time a patient receives such compositions.

It is contemplated that a pharmaceutical composition of Compound I may be used to inhibit or ameliorate prostate cancer cell growth and that a significantly improved inhibition or amelioration of prostate cancer cell growth may be obtained when castration, hormonal castration, hormonal ablation, or hormone therapy are provided before, during, and/or after the time a patient receives the compositions.

As mentioned above, although providing a subject that has cancer (e.g., prostate cancer) with a pharmaceutical composition of Compound I may inhibit the growth of cancerous cells, a significantly improved inhibition of cancer cell growth (e.g., prostate cancer cell growth) may be obtained by providing a pharmaceutical composition of Compound I, separately or in a mixture, co-administration, or combination, in conjunction with a therapy that reduces the androgen levels of the patient and/or disrupts androgen receptor signaling (e.g., castration, hormonal castration, hormonal ablation, or hormone therapy). That is, some alternatives include methods of inhibiting cancer cell growth (e.g., prostate cancer cell growth or progression of prostate cancer disease) or treating or preventing a cancer (e.g., prostate cancer), wherein a subject having a cancer (e.g., prostate cancer) is provided a pharmaceutical composition of Compound I while reducing the amount of androgens in the subject (e.g., providing castration, hormonal castration, hormonal ablation, or hormone therapy). Optionally, the inhibition of cancer (e.g., prostate cancer) or a marker thereof (e.g., PSA) is evaluated during or after the treatment (e.g., after the combination of a pharmaceutical composition of Compound I and hormone therapy is provided). Stated differently, some alternatives include a combination of a pharmaceutical composition of Compound I, formulated for administration separately or together, and an androgen deprivation therapy (e.g., castration, hormonal castration, hormonal ablation, or hormone therapy) for use in inhibiting, ameliorating or delaying the growth of prostate cancer cells or treating or preventing prostate cancer. The section below describes some of the approaches that may be used to deplete the levels of androgen in the subject so as to provide the treatments and treatment protocols described above.

C. Hormone Therapy

Hormone therapy for treating prostate cancer, or inhibiting or delaying prostate cancer cell growth, can also be called androgen deprivation therapy (ADT), chemical castration, or androgen ablation therapy. Androgens can fuel the growth of prostatic cells, including both healthy prostatic cells and cancerous prostatic cells. In some alternatives, a subject suffering from prostate cancer is provided with a hormone therapy agent that reduces the subject's androgen levels.

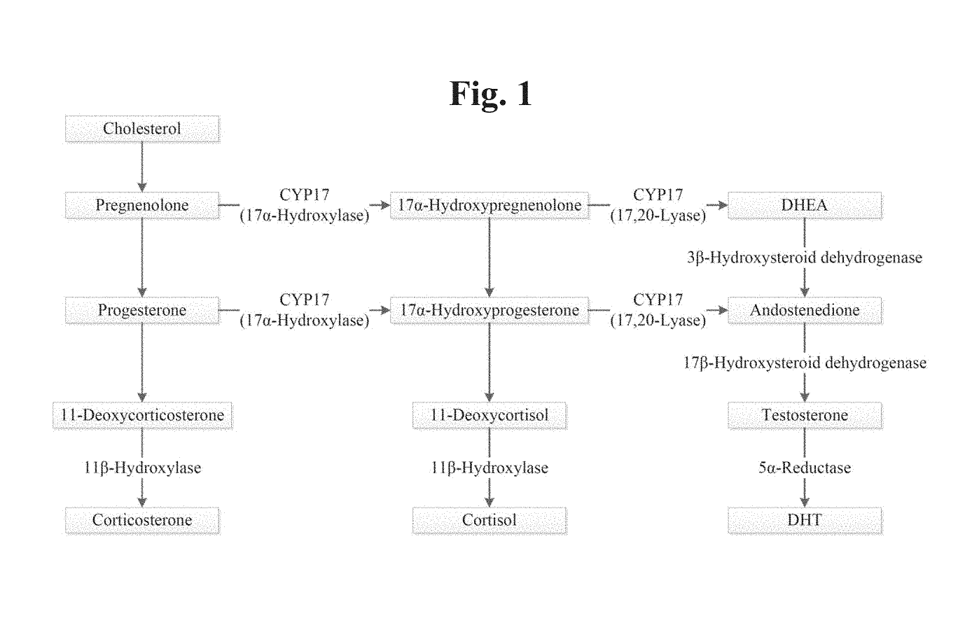

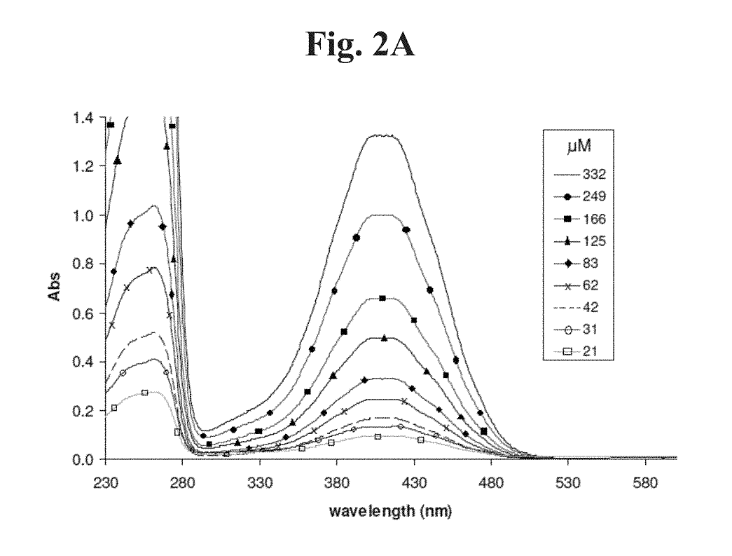

Without wishing to be bound by theory, FIG. 1 illustrates the steroid/androgen synthesis pathway. In FIG. 1, cholesterol is converted to pregnenolone, which then undergoes conversion along the mineralcortioid biosynthesis pathway to progesterone, 11-deoxycorticosterone, and corticosterone (and then to 18-hydroxycorticosterone and aldosterone, not pictured). The conversion to corticosterone occurs via the enzyme 11β-hydroxylase. 11β-hydroxylase is also featured in the glucocorticoid pathway. For the glucocorticoid biosynthesis pathway, pregnenolone or progesterone is converted via the 17α-hydroxylase activity of cytochrome P450-17 (“CYP17”) to either 17α-hydroxypregnenolone or 17α-hydroxyprogesterone. 17α-hydroxyprogesterone is converted to 11-deoxycortisol, which in turn is converted to cortisol by 11β-hydroxylase. CYP17 is also featured in the androgen biosynthesis pathway. CYP17, utilizing its 17,20-lyase activity, converts 17α-hydroxypregnenolone to dehydroepiandrosterone (“DHEA”) and 17α-hydroxyprogesterone to adostenedione. Adostenedione, in turn, is converted to testosterone by 17β-hydroxysteroid dehydrogenase, while testosterone is converted to dihydrotestosterone (“DHT”) by 5α-reductase.

In some alternatives, a hormonal therapy agent is provided to a patient to selectively inhibit the androgen biosynthesis pathway. Selective inhibition of this pathway is desirable given that a patient receiving such an agent will not require hormone replacement therapy. Hormone replacement therapy is often required when non-selective hormonal therapy agents, such as abiraterone are provided, resulting in the inhibition of mineralocorticoid biosynthesis and/or glucocorticoid biosynthesis. Such inhibition may afford side effects, causes the patient to take additional drugs, reduces patient compliance, and/or impairs the patient's quality of life.

In some alternatives, a hormonal therapy agent is provided to a patient to selectively inhibit the 17,20-lyase activity of CYP17. Such inhibition will result in the selective decrease of DHEA and andostenedione production, while not affecting mineralocorticoid biosynthesis and glucocorticoid biosynthesis. Indeed, selectivity targeting CYP17's 17,20-lyase activity, while leaving the 17α-hydroxylase activity of CYP17 relatively undisturbed should afford limited side effects and be less likely to require the concomitant administration of a hormone replacement, such as prednisone.

Inhibitors of 17,20-lyase activity of cytochrome P450-17 (“CYP-17”) are known in the art. Steroid-type inhibitors of 17,20-lyase activity are disclosed in, for example, WO 92/15404, WO 93/20097, EP-A 288053, and EP-A 413270, such compounds being incorporated herein by reference. Non-steroid-type compounds are disclosed in, for example, in WO94/27989, WO96/14090, WO97/00257; WO95/09157; U.S. Pat. No. 5,491,161; WO99/18075; WO99/54309; WO03/027085; and EP0724591, such compounds being expressly incorporated herein by reference in their entireties. Additional compounds include, but are not limited to, compounds disclosed in U.S. Pat. No. 8,236,962; U.S. Pat. No. 8,263,635; and U.S. Patent Application No. 20100305078; the compounds described therein being expressly incorporated herein by reference in their entireties.

Specific examples of selective 17,20-lyase inhibitors for use in certain alternatives include 6-(7-hydroxy-6,7-dihydro-5H-pyrrolo[1,2-c]imidazole-7-yl)-2-naphthamide; 6-(7-hydroxy-6,7-dihydro-5H-pyrrolo[1,2-c]imidazole-7-yl)-N-methyl-2-naphthamide; N-ethyl-6-(7-hydroxy-6,7-dihydro-5H-pyrrolo[1,2-c]imidazole-7-yl)-2-naphthamide; N-cyclopropyl-6-(7-hydroxy-6,7-dihydro-5H-pyrrolo[1,2-c]imidazole-7-yl)-2-naphthamide; 6-(7-hydroxy-6,7-dihydro-5H-pyrrolo[1,2-c]imidazole-7-yl)-N-isopropyl-2-naphthamide; N,N-diisopropyl-6-(7-hydroxy-6,7-dihydro-5H-pyrrolo[1,2-c]imidazole-7-yl)-2-naphthamide; 6-[1-hydroxy-1-(1-methyl-1H-imidazol-5-yl)ethyl]-N-methyl-naphthalene-2-carboxamide; 6-(6,7-dihydro-5H-pyrrolo[1,2-c]imidazole-7-yl)-N-methyl-2-naphthamide; and 6-(7-hydroxy-6,7-dihydro-6,6-dimethyl-5H-pyrrolo[1,2-c]imidazole-7-yl)-N-isopropyl-2-naphthamide. See Kaku et al., Bioorg. Med. Chem. (2011) 19, 6383-99.

Moreover, preferred examples of selective 17,20-lyase inhibitors include orteronel and VT-464. See Kaku et al., Bioorg. Med. Chem. (2011) 19, 6383-99; Eisner et al. J. Clin. Oncol. “VT-464: A novel, selective inhibitor of P450c17(CYP17)-17,20 lyase for castration-refractory prostate cancer (CRPC).

One of skill in the art can readily determine additional examples of selective 17,20-lyase inhibitors by screening inhibitors of CYP17 for both 17,20-lyase inhibition and hydroxylase inhibition, such as 17α-hydroxylase inhibition. In some alternatives, a compound is a selective inhibitor if there is a 5-fold difference between lyase and hydroxylase inhibition. In other alternatives, a selective inhibitor will have an inhibition that is at least or equal to a 10, 20, 30, 50, or 100-fold difference or any fold difference in between these numbers. Methods to determine selective inhibition are known in the art.

In some alternatives, a hormonal therapy agent is selected from the group consisting of cyproterone acetate, abiraterone, finasteride, flutamide, nilutamide, bicalutamide, diethylstilbestrol (DES), megestrol acetate, fosfestrol, estamustine phosphate, leuprolide, triptorelin, goserelin, histrelin, buserelin, abarelix, degarelix, orteronel, VT-464, enzalutamide, ARN-509, vinclozolin, galeterone, ketoconazole, L-39, aminoglutethimide, prochloraz, dutasteride, izonsteride, turosteride, epristeride, genisterin, gossypol, equol, 18β-glycyrrhetinic acid, altraric acid, N-butylbenzene-sulfonamide, 3,3′-diindolylmethane, deslorelin, nafarelin, cetrorelix, and ganirelix or any combination thereof.

In other alternatives, the hormonal therapy agent is selected from the group consisting of enzulatomide; ARN-509; vinclozolin; galeterone; ketoconazole; L-39; VT-464; orteronel; aminoglutethimide; prochloraz; dutasteride; izonsteride; turosteride; epristeride; genisterin; gossypol; equol; 18β-glycyrrhetinic acid; altraric acid; N-butylbenzene-sulfonamide; and 3,3′-diindolylmethane or any combination thereof. In other alternatives, the hormonal therapy agent is selected from the group consisting of enzalutamide; ARN-509; and vinclozolin or any combination thereof. In other alternatives, the hormonal therapy agent is selected from the group consisting of galeterone; L-39; VT-464; orteronel; aminoglutethimide; and prochloraz or any combination thereof. In other alternatives, the hormonal therapy agent is selected from the group consisting of dutasteride; izonsteride; turosteride; and epristeride or any combination thereof. In other alternatives, the hormonal therapy agent is selected from the group consisting of genisterin; gossypol; equol; 18β-glycyrrhetinic acid; altraric acid; N-butylbenzene-sulfonamide; and 3,3′-diindolylmethane or any combination thereof. In other alternatives, the hormonal therapy agent is selected from the group consisting of deslorelin; nafarelin; cetrorelix; and ganirelix or any combination thereof. In other alternatives, the hormonal therapy agent is selected from the group consisting of degarelix, abiraterone, leupron, and dutasteride.

In some alternatives, the hormonal therapy agent is a luteinizing hormone-releasing hormone (LHRH) antagonist or agonist. In some alternatives, the hormonal therapy agent is a gonadotropin-releasing hormone agonist. In some alternatives, the hormonal therapy agent is a gonadotropin-releasing hormone agonist selected from deslorelin or nafarelin or a combination thereof. In some alternatives, the hormonal therapy agent is a gonadotropin-releasing hormone antagonist. In some alternatives, the hormonal therapy agent is a gonadotropin-releasing hormone antagonist selected from cetrorelix or ganirelix or a combination thereof.

In some alternatives, one or more of the hormone therapy agents described above are administered to the patient before administering a pharmaceutical composition of Compound I. In other alternatives, one or more of the hormone therapy agents described above are administered to the patient after administering a pharmaceutical composition of Compound I. In other alternatives, one or more of the hormone therapy agents described above are concurrently (e.g., within a few minutes or hours) administered to the patient with a pharmaceutical composition of Compound I.

In some alternatives, the androgen that is decreased in the subject is testosterone, dihydrotestosterone (DHT), androsterone, androstenediol, androstenedione, dehydroepiandrosterone (DHEA), and/or dehydroepiandrosterone sulfate (DHEA-S). In some alternatives, a subject's serum testosterone level is decreased with one or more anti-androgen agents or androgen ablation agents. Preferably, the androgen deprivation therapy is provided during a period in which a pharmaceutical composition of Compound I is provided. In some alternatives, androgen deprivation therapy reduces the production of testosterone in a patient. In some embodiments, androgen deprivation therapy reduces the production of one or more hormones selected from testosterone, dihydrotestosterone (DHT), androsterone, androstenediol, androstenedione, dehydroepiandrosterone (DHEA), or dehydroepiandrosterone sulfate (DHEA-S).

In some alternatives, a subject suffering from prostate cancer is classified or identified as a subject in need of a therapy for prostate cancer and said subject is provided a hormone therapy agent that reduces the subject's androgen levels while said subject is receiving a pharmaceutical composition of Compound I. Optionally, the inhibition in prostate cancer cell growth or an inhibition in prostate cancer advancement is evaluated. Optionally, the delaying prostate cancer cell growth or delaying prostate cancer advancement is evaluated. A subject can be identified as one in need of a therapy for prostate cancer using conventional clinical pathology including, biopsy, CT scan, MRI, digital examination, Gleason score, or PSA level. A patient may receive a PET scan, which evaluate the activity of the tumor cells (glucose metabolism). Similarly, the inhibition or delay of cancer cell growth in said subject after receiving the treatment can be evaluated using conventional clinical pathology including, biopsy, CT scan, MRI, digital examination, Gleason score, or PSA level.

In some alternatives, the hormone therapy agent that can be used with any one or more of the methods or treatments described herein is selected from the group consisting of an antiandrogen (including steroidal antiandrogens and nonsteroidal antiandrogens), an estrogen, a luteinizing hormone-releasing hormone (LHRH) agonist, and a LHRH antagonist or any combination thereof. Steroidal antiandrogen agents include, but are not limited to, cyproterone acetate and/or finasteride. Nonsteroidal antiandrogens include, but are not limited to, flutamide, nilutamide and/or bicalutamide. Estrogen agents include, but are not limited to, diethylstilbestrol (DES), megestrol acetate, fosfestrol, and/or estamustine phosphate. LHRH agonist agents include, but are not limited to, leuprolide, triptorelin, goserelin, histrelin and/or buserelin. LHRH antagonist agents include, but are not limited to, abarelix and/or degarelix. Desirably, one or more of the compounds selected from the group consisting of cyproterone acetate, finasteride, flutamide, abiraterone, nilutamide, bicalutamide, diethylstilbestrol (DES), megestrol acetate, fosfestrol, estamustine phosphate, leuprolide, triptorelin, goserelin, histrelin, buserelin, abarelix, degarelix, orteronel, VT-464, enzalutamide, ARN-509, vinclozolin, galeterone, ketoconazole, L-39, aminoglutethimide, prochloraz, dutasteride, izonsteride, turosteride, epristeride, genisterin, gossypol, equol, 18β-glycyrrhetinic acid, altraric acid, N-butylbenzene-sulfonamide, 3,3′-diindolylmethane, deslorelin, nafarelin, cetrorelix, and ganirelix or any combination thereof are used in the methods and treatments (compositions) described herein, wherein a pharmaceutical composition of Compound I is provided before, during, and/or after providing said cyproterone acetate, finasteride, flutamide, abiraterone, nilutamide, bicalutamide, diethylstilbestrol (DES), megestrol acetate, fosfestrol, estamustine phosphate, leuprolide, triptorelin, goserelin, histrelin, buserelin, abarelix, degarelix, orteronel, VT-464, enzalutamide, ARN-509, vinclozolin, galeterone, ketoconazole, L-39, aminoglutethimide, prochloraz, dutasteride, izonsteride, turosteride, epristeride, genisterin, gossypol, equol, 18β-glycyrrhetinic acid, altraric acid, N-butylbenzene-sulfonamide, 3,3′-diindolylmethane, deslorelin, nafarelin, cetrorelix, or ganirelix or any combination thereof.

As mentioned above, prostate cancer can be treated by hormone therapy agents, however, hormone therapy agents alone can result in the development of castration-resistant prostate cancer (CRPC). For example, hormonal therapy can initially deliver a response in a subject suffering from prostate cancer, however, the return of hormone-refractory tumors invariably prevents long-term patient survival. More effective strategies are needed to extend life expectancy and improve the quality of life for patients with advanced prostate cancer. Accordingly, some aspects disclosed herein concern methods for ameliorating or inhibiting or reducing or delaying the onset of castration-resistant prostate cancer (CRPC) or treatments (e.g., compositions used for the purpose of ameliorating or inhibiting or reducing or delaying the onset of CRPC), whereby a pharmaceutical composition of Compound I is provided before, during and/or after providing cyproterone acetate, finasteride, abiraterone, flutamide, nilutamide, bicalutamide, diethylstilbestrol (DES), megestrol acetate, fosfestrol, estamustine phosphate, leuprolide, triptorelin, goserelin, histrelin, buserelin, abarelix, degarelix, orteronel, VT-464, enzalutamide, ARN-509, vinclozolin, galeterone, ketoconazole, L-39, aminoglutethimide, prochloraz, dutasteride, izonsteride, turosteride, epristeride, genisterin, gossypol, equol, 18β-glycyrrhetinic acid, altraric acid, N-butylbenzene-sulfonamide, 3,3′-diindolylmethane, deslorelin, nafarelin, cetrorelix, or ganirelix or any combination thereof. Optionally, the inhibition in prostate cancer cell growth, an inhibition in prostate cancer advancement, or delaying the onset of CRPC is evaluated before during or after the therapy. Optionally, a patient with prostate cancer is classified as a subject in need of an agent that ameliorates, reduces, delays, or inhibits the onset of CRPC prior to receiving one or more of the combination therapies described herein. A subject can be identified as one in need of a therapy for prostate cancer using conventional clinical pathology including, biopsy, CT scan, PET scan, MRI, digital examination, Gleason score, or PSA level.

As mentioned above, prostate cancer can be treated by hormone therapy agents, however, hormone therapy agents alone can result in damage to the prostatic capsule, including rupture of the prostate capsule, which may lead to dissemination of prostatic cancer cells outside of the capsule. More effective strategies are needed to prevent rupture of the prostatic capsule to extend life expectancy and improve the quality of life for patients with prostate cancer. Accordingly, some aspects disclosed herein concern methods for ameliorating or inhibiting or reducing or delaying damage or rupturing of the prostatic capsule or treatments (e.g., compositions used for the purpose of ameliorating or inhibiting or reducing or delaying the damage or rupturing of the prostatic capsule), whereby a pharmaceutical composition of Compound I is provided before, during and/or after providing cyproterone acetate, finasteride, abiraterone, flutamide, nilutamide, bicalutamide, diethylstilbestrol (DES), megestrol acetate, fosfestrol, estamustine phosphate, leuprolide, triptorelin, goserelin, histrelin, buserelin, abarelix, degarelix, orteronel, VT-464, enzalutamide, ARN-509, vinclozolin, galeterone, ketoconazole, L-39, aminoglutethimide, prochloraz, dutasteride, izonsteride, turosteride, epristeride, genisterin, gossypol, equol, 18β-glycyrrhetinic acid, altraric acid, N-butylbenzene-sulfonamide, 3,3′-diindolylmethane, deslorelin, nafarelin, cetrorelix, or ganirelix or any combination thereof. Optionally, a patient with prostate cancer is classified as a subject in need of an agent that ameliorates, reduces, delays, or inhibits the damage or rupturing of the prostatic capsule prior to receiving one or more of the combination therapies described herein.

D. Combination Therapies

In some alternatives, a pharmaceutical composition of Compound I disclosed herein can be used in combination with one or more hormone therapy agents. Some alternatives disclosed herein relate to a method of ameliorating or treating a neoplastic disease that can include administering or providing to a subject suffering from a neoplastic disease a pharmaceutical composition of Compound I in combination with one or more additional agents, including hormone therapy agents (referred to as “combination therapy”).

Examples of additional agents that can be used in combination with a pharmaceutical composition of Compound I include, but are not limited to, agents that can decrease the subject's serum androgen levels (e.g., cyproterone acetate, abiraterone, finasteride, flutamide, nilutamide, bicalutamide, diethylstilbestrol (DES), megestrol acetate, fosfestrol, estamustine phosphate, leuprolide, triptorelin, goserelin, histrelin, buserelin, abarelix, degarelix, orteronel, VT-464, enzalutamide, ARN-509, vinclozolin, galeterone, ketoconazole, L-39, aminoglutethimide, prochloraz, dutasteride, izonsteride, turosteride, epristeride, genisterin, gossypol, equol, 18β-glycyrrhetinic acid, altraric acid, N-butylbenzene-sulfonamide, 3,3′-diindolylmethane, deslorelin, nafarelin, cetrorelix, or ganirelix or any combination thereof).

In some embodiments, a pharmaceutical composition of Compound I disclosed herein may be used in combination with cyproterone acetate. In some embodiments, a pharmaceutical composition of Compound I disclosed herein may be used in combination with abiraterone. In some embodiments, a pharmaceutical composition of Compound I disclosed herein may be used in combination with finasteride. In some embodiments, a pharmaceutical composition of Compound I disclosed herein may be used in combination with flutamide. In some embodiments, a pharmaceutical composition of Compound I disclosed herein may be used in combination with nilutamide. In some embodiments, a pharmaceutical composition of Compound I disclosed herein may be used in combination with bicalutamide.

In some embodiments, a pharmaceutical composition of Compound I disclosed herein may be used in combination with diethylstilbestrol (DES). In some embodiments, a pharmaceutical composition of Compound I disclosed herein may be used in combination with megestrol acetate. In some embodiments, a pharmaceutical composition of Compound I disclosed herein may be used in combination with fosfestrol. In some embodiments, a pharmaceutical composition of Compound I disclosed herein may be used in combination with estamustine phosphate. In some embodiments, a pharmaceutical composition of Compound I disclosed herein may be used in combination with leuprolide. In some embodiments, a pharmaceutical composition of Compound I disclosed herein may be used in combination with triptorelin. In some embodiments, a pharmaceutical composition of Compound I disclosed herein may be used in combination with goserelin. In some embodiments, a pharmaceutical composition of Compound I disclosed herein may be used in combination with histrelin. In some embodiments, a pharmaceutical composition of Compound I disclosed herein may be used in combination with buserelin.

In some embodiments, a pharmaceutical composition of Compound I disclosed herein may be used in combination with abarelix. In some embodiments, a pharmaceutical composition of Compound I disclosed herein may be used in combination with degarelix. In some embodiments, a pharmaceutical composition of Compound I disclosed herein may be used in combination with orteronel. In some embodiments, a pharmaceutical composition of Compound I disclosed herein may be used in combination with VT-464. In some embodiments, a pharmaceutical composition of Compound I disclosed herein may be used in combination with enzalutamide. In some embodiments, a pharmaceutical composition of Compound I disclosed herein may be used in combination with ARN-509. In some embodiments, a pharmaceutical composition of Compound I disclosed herein may be used in combination with vinclozolin. In some embodiments, a pharmaceutical composition of Compound I disclosed herein may be used in combination with galeterone. In some embodiments, a pharmaceutical composition of Compound I disclosed herein may be used in combination with ketoconazole. In some embodiments, a pharmaceutical composition of Compound I disclosed herein may be used in combination with L-39.

In some embodiments, a pharmaceutical composition of Compound I disclosed herein may be used in combination with aminoglutethimide. In some embodiments, a pharmaceutical composition of Compound I disclosed herein may be used in combination with prochloraz. In some embodiments, a pharmaceutical composition of Compound I disclosed herein may be used in combination with dutasteride. In some embodiments, a pharmaceutical composition of Compound I disclosed herein may be used in combination with izonsteride. In some embodiments, a pharmaceutical composition of Compound I disclosed herein may be used in combination with turosteride. In some embodiments, a pharmaceutical composition of Compound I disclosed herein may be used in combination with epristeride.

In some embodiments, a pharmaceutical composition of Compound I disclosed herein may be used in combination with genisterin. In some embodiments, a pharmaceutical composition of Compound I disclosed herein may be used in combination with gossypol. In some embodiments, a pharmaceutical composition of Compound I disclosed herein may be used in combination with equol. In some embodiments, a pharmaceutical composition of Compound I disclosed herein may be used in combination with 18β-glycyrrhetinic acid. In some embodiments, a pharmaceutical composition of Compound I disclosed herein may be used in combination with altraric acid. In some embodiments, a pharmaceutical composition of Compound I disclosed herein may be used in combination with N-butylbenzene-sulfonamide. In some embodiments, a pharmaceutical composition of Compound I disclosed herein may be used in combination with 3,3′-diindolylmethane. In some embodiments, a pharmaceutical composition of Compound I disclosed herein may be used in combination with deslorelin. In some embodiments, a pharmaceutical composition of Compound I disclosed herein may be used in combination with nafarelin. In some embodiments, a pharmaceutical composition of Compound I disclosed herein may be used in combination with cetrorelix. In some embodiments, a pharmaceutical composition of Compound I disclosed herein may be used in combination with ganirelix.

In some embodiments, a pharmaceutical composition of Compound I disclosed herein may be used in combination with a hormone therapy agent that suppress androgen receptor axis in prostate cancer. The hormone therapy agent may suppress the androgen receptor axis in prostate cancer by suppressing the circulation of androgen. The hormone therapy agent may suppress the androgen receptor axis in prostate cancer by inhibiting androgen receptors.

In some embodiments, a pharmaceutical composition of Compound I disclosed herein may be used in combination with a bipolar androgen therapy (“BAT”). In bipolar androgen therapy, a subject is provided with a hormone therapy agent or surgical castration that suppresses endogenous androgen levels in the subject throughout the duration of the biopolar androgen therapy. The subject is further provided with at least one dosage of exogenous androgen that produces supraphysiologic androgen levels in the subject for a first period of time within the duration of the biopolar androgen therapy. After the at least one dosage of exogenous androgen, the subject's androgen levels are permitted to return to the suppressed levels caused by the hormone therapy agent or surgical castration for a second period of time. An exemplary cycle of biopolar androgen therapy includes (1) providing a subject with a daily dosage of hormone therapy agent for 28 days, and (2) on day 1 of the 28 days, providing the subject with a dosage of exogenous androgen that produces supraphysiologic androgen levels in the subject. In some embodiments, the supraphysiologic androgen levels produced in the subject by the dosage of exogenous androgen can cause the subject's testosterone serum levels to range between 1000-5000 ng/dL. In some embodiments, the supraphysiologic androgen levels produced in the subject by the dosage of exogenous androgen can cause the subject's serum testosterone level to be at least or any number in between the range of 750-850, 800-900, 850-950, 900-1000, 950-1050, 1000-1500, 1250-1750, 1500-2000, 1750-2250, 2000-3000, 2500-3500, 3000-4000, 3500-4500, or 4000-5000 ng/dL or within a range defined by any two of the aforementioned values. In some embodiments, the supraphysiologic androgen levels produced in the subject by the dosage of exogenous androgen can cause the subject's serum testosterone level to be greater than 1000, greater than 1500, greater than 2000, greater than 2500, greater than 3000, greater than 4000, or greater than 5000 ng/dL. In some embodiments, the exogenous androgen that produces supraphysiologic androgen levels in the subject can be testosterone cypionate, testosterone enanthate, testosterone acetate, testosterone propionate, testosterone phenylpropionate, testosterone isocaproate, testosterone caproate, testosterone decanoate, testosterone undecanoate, sustanon, omnadren, methyltesosterone, or an aqueous testosterone suspension, or any combination thereof. In some embodiments, the dosage of exogenous androgen that produces supraphysiologic androgen levels in the subject can be a dosage of at least or any number in between the range of 100-1,000 mg, 100-300 mg, 200-400 mg, 300-500 mg, 400-600 mg, 500-700 mg, 600-800 mg, 700-900 mg, or 800-1,000 mg. In some embodiments, the dosage of exogenous androgen that produces supraphysiologic androgen levels in the subject can be a dosage of greater than 100 mg, greater than 300 mg, greater than 500 mg, greater than 700 mg, greater than 900 mg, or greater than 1,000 mg. A subject may be treated with a pharmaceutical composition of Compound I disclosed herein in combination with one, two, three, four, five, six, or more than six cycles of biopolar androgen therapy.

In some alternatives, the neoplastic disease can be cancer. In some alternatives, the neoplastic disease can be a tumor such as a solid tumor or metastasis. In an alternative, the neoplastic disease can be prostate cancer, such as stage I, stage II, stage III or stage IV prostate cancer and in some alternatives the prostate cancer can be CRPC, prostate cancer that has extended beyond the outer condensed fibromuscular band, also known as the capsule, or metastasis stemming from prostate cancer. In some alternatives, the prostate cancer is androgen dependent. Therefore, in some alternatives, a pharmaceutical composition of Compound I disclosed herein is used in combination with one or more hormone therapy agents for the use in treating, inhibiting, delaying, or ameliorating progression of prostate cancer, such as stage I, stage II, stage III or stage IV prostate cancer growth of prostate cancer cells, or for inhibiting or preventing the onset of androgen-dependent prostate cancer, or for decreasing the size of a prostate tumor, or for inhibiting the onset of metastasis associated with prostate cancer. In some alternatives, a pharmaceutical composition of Compound I disclosed herein is used in combination with one or more hormone therapy agents for the use in increasing the survival rate of a patient suffering from prostate cancer. In some alternatives, a pharmaceutical composition of Compound I disclosed herein is used in combination with one or more hormone therapy agents for the use in treating, inhibiting, delaying, or ameliorating damage or rupture of the prostatic capsule.

In some alternatives, a pharmaceutical composition of Compound I disclosed herein is used in combination with surgical orchiectomy and/or one or more of the hormone therapy agents (e.g. cyproterone acetate, finasteride, abiraterone, flutamide, nilutamide, bicalutamide, diethylstilbestrol (DES), megestrol acetate, fosfestrol, estamustine phosphate, leuprolide, triptorelin, goserelin, histrelin, buserelin, abarelix, degarelix, orteronel, VT-464, enzalutamide, ARN-509, vinclozolin, galeterone, ketoconazole, L-39, aminoglutethimide, prochloraz, dutasteride, izonsteride, turosteride, epristeride, genisterin, gossypol, equol, 18β-glycyrrhetinic acid, altraric acid, N-butylbenzene-sulfonamide, 3,3′-diindolylmethane, deslorelin, nafarelin, cetrorelix, or ganirelix or any combination thereof) for the use in increasing the survival rate of a patient suffering from CRPC. In some alternatives, a pharmaceutical composition of Compound I disclosed herein is used in combination with one or more hormone therapy agents for the use in reducing the size of a tumor or further expansion of cancer cells in a patient suffering from prostate cancer, such as stage I, stage II, stage III or stage IV prostate cancer. Some alternatives involve methods for inducing remission of prostate cancer, such as stage I, stage II, stage III or stage IV prostate cancer, whereby a pharmaceutical composition of Compound I disclosed herein is provided before, during and/or after providing a hormone therapy agent to a subject suffering from prostate cancer. In some alternatives, the methods disclosed herein can result in complete remission of prostate cancer, such as stage I, stage II, stage III or stage IV prostate cancer. In some alternatives, the methods can result in partial remission of prostate cancer, such as stage I, stage II, stage III or stage IV prostate cancer.

In some embodiments, administration of a pharmaceutical composition of Compound I to a subject according to any one of the methods disclosed herein results in survival of the subject for at least 1 month, 2 months, 3 months, 6 months, 9 months, 12 months, 15 months, 18 months, 24 months, 36 months, or 48 months or within a range defined by any two of the aforementioned times. In some embodiments, administration of a pharmaceutical composition of Compound I to a subject according to any one of the methods disclosed herein results in survival of the subject for more than 48 months. In some embodiments, administration of a pharmaceutical composition of Compound I to a subject according to any one of the methods disclosed herein results in survival of the subject for more than 60 months.

Normal serum testosterone ranges between 1000-300 ng/dL. In some alternatives, a subject is provided a combination therapy, as described herein, whereby a reduction in the treated subject's serum testosterone level to at least ≤80, ≤70, ≤60, ≤50, ≤40, ≤30, ≤20, or ≤10 ng/dL is obtained. In some alternatives, a subject is provided a combination therapy that reduces the subject's serum testosterone level to at least ≤50 ng/dL. In some alternatives, a subject is treated with a combination therapy that results in a reduction in the subject's serum testosterone level to at least ≤20 ng/dL. In some alternatives, a subject is treated with a combination therapy, as described herein, that reduces the subject's serum testosterone level to at least or any number in between the range of 120-70, 100-60, 80-40, 70-30, 50-20, 40-10, 30-10, or 20-10 ng/dL or within a range defined by any two of the aforementioned values. In some alternatives, a subject is treated with a combination therapy that produces a reduction in the subject's serum testosterone level to ≤95%, ≤90%, ≤80%, ≤70%, ≤60%, or ≤50% that of a healthy male or within a range defined by any two of the aforementioned values. In some alternatives, a subject is treated with a combination therapy that results in a reduction in the subject's serum testosterone level to the range of at least or any number in between the range of 5-20%, 10-30%, 20-40%, 30-50%, 40-60%, or 50-70% that of a healthy male or within a range defined by any two of the aforementioned values. In some alternatives, a subject is treated with a combination therapy that results in a reduction in the subject's serum testosterone level to the range of at least or any number in between the range of 1-2%, 2-4%, 1-5%, 4-6%, 4-8%, or 5-10% that of a healthy male or within a range defined by any two of the aforementioned values.

Intermittent hormonal therapy (IHT) is an alternative to continuous hormonal therapy, which may delay progression of hormone-refractory disease (i.e., CRPC). For example, intermittent therapy can be used for a period of 6 months on, followed by a period of 6 months off. In some alternatives, one or more hormonal therapy agents is provided for one month on, followed by one month off. In some alternatives, one or more therapy agents are provided for three months on, followed by three months off. Accordingly, a pharmaceutical composition of Compound I disclosed herein can be provided before, during and/or after administering one or more hormonal therapy agents, as described above, so as to reduce or inhibit or delay the onset of CRPC.

The order of administration of a pharmaceutical composition of Compound I disclosed herein with one or more additional hormone therapy agent(s) can vary. In some alternatives, a pharmaceutical composition of Compound I disclosed herein can be administered prior to all additional hormone therapy agents. In other alternatives, a pharmaceutical composition of Compound I disclosed herein can be administered prior to at least one additional hormone therapy agent. In still other alternatives, a pharmaceutical composition of Compound I disclosed herein can be administered concomitantly with one or more additional hormone therapy agent(s). In yet still other alternatives, a pharmaceutical composition of Compound I disclosed herein can be administered subsequent to the administration of at least one additional hormone therapy agent. In some alternatives, a pharmaceutical composition of Compound I disclosed herein can be administered subsequent to the administration of all additional hormone therapy agents.

In some alternatives, a subject suffering from prostate cancer is provided surgical orchiectomy (i.e., removal of the testes). In some alternatives, a pharmaceutical composition of Compound I disclosed herein can be administered after surgical orchiectomy. In some alternatives, a pharmaceutical composition of Compound I disclosed herein can be administered before and after surgical orchiectomy.

In some alternatives, a pharmaceutical composition of Compound I disclosed herein can be used in combination with one or more hormone therapy agents and in further combination with one or more statins. Statins are inhibitors of HMG-CoA reductase that can be administered to a subject to reduce testosterone/dihydrotestosterone levels. In some alternatives, a pharmaceutical composition of Compound I disclosed herein can be used in combination with one or more statins. In some alternatives, the one or more statins can be selected from among simvastatin (Zocor), atrovastatin (Lipitor), fluvastatin (Lescol), lovastatin (Mevacor, Altocor), pitavastatin (Livalo), pravastatin (Pravachol), or rosuvastatin (Crestor) or any combination thereof.

Some alternatives described herein relate to a pharmaceutical composition that can include a therapeutically effective amount of Compound I, a hormone therapy agent, and at least one pharmaceutically acceptable carrier. Some alternatives described herein relate to a pharmaceutical composition that can include a therapeutically effective amount of Compound I, a hormone therapy agent, at least one pharmaceutically acceptable carrier, and at least one excipient.

### VI. Dosage Amounts of Compound I

In some embodiments, the pharmaceutical composition of Compound I disclosed herein may contain between 0.01 mg and 3000 mg of Compound I, preferably between 1 mg and 700 mg, e.g. 5 to 200 mg. In non-human animal studies, applications of potential products are commenced at higher dosage levels, with dosage being decreased until the desired effect is no longer achieved or adverse side effects disappear. The LD₅₀ for a suspension of Compound I in 0.5% of gum acacia in water or in propylene glycol for oral administration in rat was 65 mg/kg. In mice, the LD₅₀ was 16 to 40 mg/kg per oral over a period of 72 hours or 8 mg/kg over a period of 14 days. It was determined that the maximum tolerated dose of Compound I in a pharmaceutical composition with pharmaceutically acceptable Carrier A was 100 mg/kg (see, supra Example 6). Very little toxicity was observed in rats at 10 mg/ml. It was determined that Compound I was maximally effective at doses of 1 mg/kg, well below toxic doses, and therefore appears to have a very good therapeutic index (see, supra Example 5).

In some embodiments, a pharmaceutical composition of Compound I contains an amount of Compound I of 10 mg, or 20 mg, or 30 mg, or 35 mg, or 40 mg, or 45 mg, or 50 mg, or 55 mg, or 60 mg, or 65 mg, or 70 mg, or 75 mg, or 80 mg, or 85 mg, or 90 mg, or 95 mg, or 100 mg, or 110 mg, or 120 mg, or 130 mg, or 140 mg, or 150 mg, or 160 mg, or 170 mg, or 180 mg, or 190 mg, or 200 mg, or 210 mg, or 220 mg, or 230 mg, or 240 mg, or 250 mg, or 260 mg, or 270 mg, or 280 mg, or 290 mg, or 300 mg, or 325 mg, or 350 mg, or 375 mg, or 400 mg, or 425 mg, or 450 mg, or 475 mg, or 500 mg, or 525 mg, or 550 mg, or 575 mg, or 600 mg, or 650 mg, or 700 mg, or 750 mg, or 800 mg, or 850 mg, or 900 mg, or 950 mg, or 1000 mg, or 1100 mg, or 1200 mg, or 1300 mg, or 1400 mg, or 1500 mg or an amount that is within a range defined by any two of the aforementioned amounts.

In some embodiments, a pharmaceutical composition of Compound I contains an amount of Compound I ranging from or any number in between 2-7 mg, 5-10 mg, 7-12 mg, 10-15 mg, 12-17 mg, 15-20 mg, 17-22 mg, 20-25 mg, 22-27 mg, 25-30 mg, 27-32 mg, 30-35 mg, 32-37 mg, 35-40 mg, 37-42 mg, 40-45 mg, 40-50 mg, 45-55 mg, 50-60 mg, 55-65 mg, 60-70 mg, 65-75 mg, 70-80 mg, 75-85 mg, 80-90 mg, 85-95 mg, 90-100 mg, 95-105 mg, 100-120 mg, 110-130 mg, 120-140 mg, 130-150 mg, 140-160 mg, 150-170 mg, 160-180 mg, 170-190 mg, 180-200 mg, 190-210 mg, 200-240 mg, 220-260 mg, 240-280 mg, 260-300 mg, 280-320 mg, 300-350 mg, 325-375 mg, 350-400 mg, 375-425 mg, 400-450 mg, 425-475 mg, 450-500 mg, 475-525 mg, 500-600 mg, 550-650 mg, 600-700 mg, 650-750 mg, 700-800 mg, 750-850 mg, 800-900 mg, 850-950 mg, 900-1000 mg, 950-1150 mg, 1000-1200 mg, 1100-1300 mg, 1200-1400 mg, or 1300-1500 mg or an amount that is within a range defined by any two of the aforementioned amounts.

In some embodiments, a pharmaceutical composition of Compound I contains an amount of Compound I of 0.25 mg per kg body weight of the subject, 0.30 mg per kg body weight of the subject, 0.35 mg per kg body weight of the subject, 0.40 mg per kg body weight of the subject, 0.45 mg per kg body weight of the subject, 0.50 mg per kg body weight of the subject, 0.55 mg per kg body weight of the subject, 0.60 mg per kg body weight of the subject, 0.65 mg per kg body weight of the subject, 0.70 mg per kg body weight of the subject, 0.75 mg per kg body weight of the subject, 0.80 mg per kg body weight of the subject, 0.85 mg per kg body weight of the subject, 0.90 mg per kg body weight of the subject, 0.95 mg per kg body weight of the subject, 1.0 mg per kg body weight of the subject, 1.1 mg per kg body weight of the subject, 1.2 mg per kg body weight of the subject, 1.3 mg per kg body weight of the subject, 1.4 mg per kg body weight of the subject, 1.5 mg per kg body weight of the subject, 1.6 mg per kg body weight of the subject, 1.7 mg per kg body weight of the subject, 1.8 mg per kg body weight of the subject, 1.9 mg per kg body weight of the subject, 2 mg per kg body weight of the subject, 3 mg per kg body weight of the subject, 4 mg per kg body weight of the subject, 5 mg per kg body weight of the subject, 6 mg per kg body weight of the subject, 7 mg per kg body weight of the subject, 8 mg per kg body weight of the subject, 9 mg per kg body weight of the subject, 10 mg per kg body weight of the subject, 15 mg per kg body weight of the subject, 20 mg per kg body weight of the subject, 25 mg per kg body weight of the subject, 30 mg per kg body weight of the subject, 35 mg per kg body weight of the subject, 40 mg per kg body weight of the subject, 45 mg per kg body weight of the subject, 50 mg per kg body weight of the subject, 60 mg per kg body weight of the subject, 70 mg per kg body weight of the subject, 80 mg per kg body weight of the subject, 90 mg per kg body weight of the subject, or 100 mg per kg body weight of the subject or an amount that is within a range defined by any two of the aforementioned amounts.

In some embodiments, a pharmaceutical composition of Compound I contains an amount of Compound I ranging from or any number in between 0.20-0.25 mg per kg body weight of the subject, 0.22-027 mg per kg body weight of the subject, 0.25-0.30 mg per kg body weight of the subject, 0.27-0.32 mg per kg body weight of the subject, 0.30-0.35 mg per kg body weight of the subject, 0.32-0.37 mg per kg body weight of the subject, 0.35-0.40 mg per kg body weight of the subject, 0.37-0.42 mg per kg body weight of the subject, 0.40-0.45 mg per kg body weight of the subject, 0.40-0.5 mg per kg body weight of the subject 0, 0.45-0.55 mg per kg body weight of the subject, 0.50-0.60 mg per kg body weight of the subject, 0.55-0.65 mg per kg body weight of the subject, 0.60-0.70 mg per kg body weight of the subject, or 0.65-0.75 mg per kg body weight of the subject, 0.70-0.80 mg per kg body weight of the subject, or 0.75-0.85 mg per kg body weight of the subject, 0.80-0.90 mg per kg body weight of the subject, or 0.85-0.95 mg per kg body weight of the subject, 0.90-1.00 mg per kg body weight of the subject, or 0.95-1.15 mg per kg body weight of the subject, 1.0-1.2 mg per kg body weight of the subject, or 1.1-1.3 mg per kg body weight of the subject, 1.2-1.4 mg per kg body weight of the subject, or 1.3-1.5 mg per kg body weight of the subject, 1.4-1.6 mg per kg body weight of the subject, or 1.5-1.7 mg per kg body weight of the subject, 1.6-1.8 mg per kg body weight of the subject, or 1.7-1.9 mg per kg body weight of the subject, 1.8-2.0 mg per kg body weight of the subject, or 1.9-2.1 mg per kg body weight of the subject, 1-3 mg per kg body weight of the subject, or 2-4 mg per kg body weight of the subject, 3-5 mg per kg body weight of the subject, or 4-6 mg per kg body weight of the subject, 5-7 mg per kg body weight of the subject, or 6-8 mg per kg body weight of the subject, 7-9 mg per kg body weight of the subject, or 8-10 mg per kg body weight of the subject, 9-11 mg per kg body weight of the subject, 10-20 mg per kg body weight of the subject, 15-25 mg per kg body weight of the subject, 20-30 mg per kg body weight of the subject, 25-35 mg per kg body weight of the subject, 30-40 mg per kg body weight of the subject, 35-45 mg per kg body weight of the subject, 40-50 mg per kg body weight of the subject, 45-55 mg per kg body weight of the subject, 50-70 mg per kg body weight of the subject, 60-80 mg per kg body weight of the subject, 70-90 mg per kg body weight of the subject, or 80-100 mg per kg body weight of the subject or an amount that is within a range defined by any two of the aforementioned amounts.

In some embodiments, the amount of Compound I may vary from or any number in between 10% to 75% by weight of the total pharmaceutical composition. In some embodiments, the amount of Compound I ranges from or any number in between 10-15%, 12-17%, 15-20%, 17-22%, 25%-30%, 27%-32%, 30%-35%, 32%-37%, 35%-40%, 37%-42%, 40%-45%, 42%-47%, 45%-50%, 47%-52%, 50%-55%, 52%-57%, 55%-60%, 67%-72%, or 70%-75% by weight of the total pharmaceutical composition or an amount that is within a range defined by any two of the aforementioned amounts. In some embodiments, the amount of Compound I is at least 5%, at least 10%, at least 15%, at least 20%, at least 25%, at least 30%, at least 35%, at least 40%, at least 45%, at least 50%, at least 55%, at least 60%, at least 65%, at least 70%, or at least 75%, of the weight of the total pharmaceutical composition or an amount that is within a range defined by any two of the aforementioned amounts.

The dosage may be a single one or a series of two or more given in the course of one or more days, as is needed by the subject. In some alternatives, a pharmaceutical composition of Compound I disclosed herein will be administered for a period of continuous therapy, for example for a week or more, or for months or years. In some alternatives, a pharmaceutical composition of Compound I disclosed herein can be administered one time per day.

Multiple doses of Compound I can be administered to a subject. For example, a pharmaceutical composition of Compound I disclosed herein can be administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from one day to one week, from two weeks to four weeks, from one month to two months, from two months to four months, from four months to six months, from six months to eight months, from eight months to 1 year, from 1 year to 2 years, or from 2 years to 4 years, or more.

In some alternatives, a pharmaceutical composition of Compound I disclosed herein and a hormone therapy agent can be cyclically administered to a patient. Cycling therapy involves the administration of a first active ingredient for a period of time, followed by the administration of a second active ingredient for a period of time and repeating this sequential administration. Cycling therapy can reduce the development of resistance to one or more therapies, avoid or reduce the side effects of one or more therapies, and/or improve the efficacy of treatment. In some alternatives, a pharmaceutical composition of Compound I disclosed herein and a hormone therapy agent are administered in a cycle of less than 3 weeks, once every two weeks, once every 10 days, or once every week. The number of cycles can be from 1 to 12 cycles, or from 2 to 10 cycles, or from 2 to 8 cycles.

The daily dosage regimen for an adult human patient may be the same or different for two active ingredients provided in combination. In some alternatives, the active ingredient is Compound I. In some alternatives, the active ingredient is a hormone therapy agent. In some alternatives, both an active ingredient of Compound I and an active ingredient of a hormone therapy agent are administered to a subject. For example, Compound I can be provided at a dose of between 0.01 mg and 3000 mg, while a hormone therapy agent can be provided at a dose of between 1 mg and 700 mg. The dosage or each active ingredient can be, independently, a single one or a series of two or more given in the course of one or more days, as is needed by the subject. In some alternatives, the active ingredients will be administered for a period of continuous therapy, for example for a week or more, or for months or years. In some alternatives, a pharmaceutical composition of Compound I disclosed herein can be administered one time per day. In some alternatives, the hormone therapy agent can be administered once a week.

In instances where human dosages for active ingredients have been established for at least some condition, those same dosages may be used, or dosages that are between 0.1% and 500%, more preferably between 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

As will be understood by those of skill in the art, in certain situations it may be necessary to administer the active ingredients disclosed herein in amounts that exceed, or even far exceed, the above-stated, preferred dosage range in order to effectively and aggressively treat particularly aggressive diseases.

Dosage amount and interval may be adjusted individually to provide plasma levels of the active moiety which are sufficient to maintain the modulating effects, or minimal effective concentration (MEC). The MEC will vary for each active ingredient but can be estimated from in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

Active ingredients disclosed herein can be evaluated for efficacy and toxicity using known methods. For example, the toxicology of a particular active ingredient, or of a subset of the active ingredients, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular active ingredient may be established using several recognized methods, such as in vitro methods, animal models, or human clinical trials. When selecting a model to determine efficacy, the skilled artisan can be guided by the state of the art to choose an appropriate model, dose, route of administration and/or regime.

The toxicology of a pharmaceutical composition containing Compound I may be established by determining in vitro toxicity towards a cell line, such as a mammalian, and preferably human, cell line. The results of such studies are often predictive of toxicity in animals, such as mammals, or more specifically, humans. The toxicity of a pharmaceutical composition containing Compound I may be established by determining in vivo toxicity in an animal model, such as mice, rats, rabbits, or monkeys.

### EXAMPLES

Additional alternatives are disclosed in further detail in the following examples, which are not in any way intended to limit the scope of the claims.

### General Procedures and Methods

### Mouse Models

All procedures involving mice were approved by the Institutional Animal Care and Use Committee of Explora Biolabs (San Diego, Calif.) and carried out according to NIH recommended procedures and precautions. All mice were purchased from Jacksons laboratory (Bar Harbor, Me.). Mice were housed at a maximum of two per cage, under standard room conditions, with ad libidum food and water. Every effort was made to minimize animal suffering.

### Surgical Techniques

Mice were anesthetized before surgery and received analgesics after surgery according to IACUC-approved procedures. Surgeries were performed in a sterile laminar flow hood with proper instrument sterilization and approved procedures.

### Dorsal Skinfold Chamber, Prostate Tissue Graft, and Tumor Cell Spheroids

Dorsal skinfold chambers and surgical instruments are autoclaved before use. Saline used to keep tissue moist during surgical preparation is mixed with gentamicin (50 μl/ml).

Male Nude mice (25-35 g body weight) are anesthetized (7.3 mg ketamine hydrochloride and 2.3 mg xylazine/100 g body weight, i.p.) and placed on a heating pad. Two symmetrical titanium frames are implanted into a dorsal skinfold, so as to sandwich the extended double layer of skin. A 15 mm full thickness circular layer is excised. The underlying muscle (M. cutaneous max.) and subcutaneous tissues are covered with a glass coverslip incorporated in one of the frames. After a recovery period of 2-3 days, prostate tissue and cancer cell spheroids are carefully placed in the chamber. Small circular Band Aids are applied on the backside of the chamber after surgery to prevent scratching. Before surgery, Buprenorphine (0.1 mg/kg) is given IP. After surgery Meloxicam is given in the drinking water for 4 days Meloxicam (5.0 mg/ml), is added at 35 μl per 100 ml of water to be medicated.

### Surgical Castration

Mice are anesthetized with 7.3 mg ketamine hydrochloride and 2.3 mg xylazine/100 g body weight, i.p. A lateral incision across the scrotum is made and the testes are individually ligated and excised. The wound is cauterized. The incision is then sutured and sealed with Nexaband® acrylic.

### Cell Culture

PTEN-P2 and PTEN-CaP2 mouse prostate cancer cells were generously provided by the laboratory of Dr. Hong Wu as described in Jiao et al., Cancer Research, 67:6083-91 (2007). Expression of histone H2B-GFP fluorescent protein in PTEN-P2 and PTEN-CaP2 cells was achieved by infection with a viral vector followed by selection in geneticin.

TRAMP-C2/H2B-GFP cells were grown in phenol-free RPMI containing 10% FBS, 2 mM L-glutamine, 100 U/ml penicillin/100 μg/ml streptomycin, insulin-selenium-transferrin (10 μg/ml insulin), DHT 10⁻⁸M final and G418 (100 μg/ml). PTEN-CaP2/H2B-GFP and PTEN-P2/H2B-GFP were grown in phenol-free, high-glucose DMEM (Dulbecco's Modified Eagle's Medium) containing the same additives as TRAMP-C2 cells. G418 at 100 μg/ml or 50 μg/ml was added to maintain stable expression of H2B-GFP in PTEN-P2 and PTEN-CaP2, respectively.

Preparation of tumor spheroids: Liquid overlay plates were generated using 1% molecular biology grade agarose melted in cell culture medium and plated in round bottom 96 well plates at 50 ul/well. Tumor cells grown as pre-confluent monolayers were trypsinized and diluted to a final volume of 1,000,000 cells/ml. Viability was determined by Trypan blue. Cells were placed onto the cooled agarose (70 μl/well) and allowed to compact into spheroids for 24 hours. The spheroids were picked with a pipette and transferred into serum-free media for implantation in the chambers.

### IntraVital Microscopy (IVM)

IntraVital Microscopy (IVM) can be used to visualize tumors in animals and analyze various aspects of cancer physiology such as tumor vascularization, cell migration and metastasis. An advantage of IVM includes the real-time analysis of dynamic processes with single-cell resolution. IntraVital microscopy offers the possibility to follow tumor growth in a non-invasive, non-destructive manner. The application of IVM can be limited to animal models that bear visually accessible tumors. Therefore, the dorsal skinfold chamber model described above can be compatible with IVM. Using IVM can permit a number of parameters to be measured in living animals and as a function of time, including tumor growth, angiogenesis, infiltration by immune cells, tumor cell migration, mitosis (cell-division) and apoptosis (programmed cell death), all in the context of the host and in real time.

### Survival Studies

Orthotopic injection of PTEN-P2 and TRAMP-C2 for survival studies: Mice were anaesthetized and an incision was made through the skin and peritoneum. A total of 30,000 cells were suspended in Matrigel and slowly injected into the dorsal lobe of the prostate using a 30-gauge needle. Matrigel was used to prevent the leakage of cells out of the site of injection. A Q-tip was held over the injection site for 1 min to prevent bleeding and spill after removal of the needle. The abdominal wall was closed by surgical silk sutures.

Fox1/Nu or C57BL/6J mice were used as recipients for PTEN-P2 and TRAMP-C2 cells, respectively. Mice were surgically castrated 6 weeks later, when tumors were established, and Compound I treatment was initiated 4-5 days later. The number of days mice lived following treatment initiation was recorded for each animal. A few mice were found dead upon daily inspection, while others were euthanized in agreement with our IACUC regulations, including mice showing weight losses of more than 15% and mice showing signs of pain though posture and lack of grooming were euthanized.

### Compound I Administration

Unless otherwise indicated, Compound I was administered by oral gavage in a sesame oil (Carrier A) formulation. Compound I was dissolved in DMSO then in oil at a concentration of 0.25 mg/ml (weight/volume) with a final DMSO concentration of 0.5% (vol/vol), and administered to mice at 1 mg/kg. In some experiments, Compound I dissolved in a mixture of poly-ethylene glycol/DMSO 30% (w/v) was administered via intra-peritoneal injection.

### Toxicology Studies

A single-dose maximum tolerated dose (MTD) phase A study and a 7-day dose range-finding (DRF) phase B study were conducted in Sprague Dawley rats by MPI Research, Inc. (Mattawan, Mich.). A total of 62 males and 62 females rats were assigned to the study, which was performed in accordance with all current regulations. Animals were housed in standard conditions and had ad libitum access to food and water. Healthy animals of each sex were randomized into treatment groups using a standard, by weight, measured value randomization procedure. Compound I was administered by oral gavage, once at each dose level in Phase A (acute), or once daily for 7 consecutive days in Phase B. Oral gavage was chosen because the oral route is the intended route of administration in humans. Animals were observed twice daily for morbidity and mortality and were euthanatized for humane reasons where appropriate. An initial starting dose of 50 mg/kg was used for Phase A. Subsequent doses were increased or decreased based on the response to the preceding dose. The maximum tolerated dose (MTD) was identified as the highest dose level that did not produce mortality, more than a 10% decrement in body weight, or clinical signs of toxicity. The dose levels used in Phase B were based on the MTD identified in Phase A.

### Statistical Analysis

Means of tumor sizes within treatment groups and standard errors were calculated. The statistical significance of differences within treatment arms (i.e. efficacy of a treatment as a function of time) was calculated using one-way repeated-measures ANOVA with post hoc Bonferroni correction. The statistical significance of differences between treatment arms was calculated using two-way repeated-measures ANOVA with post hoc Bonferroni correction. The difference between survival curves was calculated using the non-parametric log-rank test.

### Example 1

### Solubility of Compound I in Aqueous Buffers and Other Solvents

The maximum quantity of Compound I that can be completely dissolved at 25° C. in the several aqueous buffers and other solvents was evaluated using HPLC. All solubility samples were saturated with a similar amount of excess Compound I. Samples were shaken overnight at 25° C., placed in 0.22 μm nylon centrifuge tube filters and centrifuged @ 14000 rpm for 10 minutes prior to HPLC analysis. Samples were injected with a 250 μg/ml nominal concentration. For samples of unknown concentration, dilutions were made to target the linear range of the method. All samples were quantified based on a linear standard curve. Measurements were performed by Pharmatek Laboratories, Inc. (San Diego, Calif.) with industry-standard quality controls. As shown in Tables 1 and 2, it was found that Compound I is mostly insoluble in aqueous media but has good solubility in organic solvents such as alcohols, acetone and other solvents. It was found that Compound I was well soluble in Carrier A (Table 2).

TABLE 1
Aqueous Thermodynamic Solubility

| | Maximum Compound I concentration |
|---|---|
| Solvent | (mg/mL) |
| 50 mM Phosphate pH 1 | 0.048 |
| 50 mM Phosphate pH 2 | 0.045 |
| 50 mM Citrate pH 3 | 0.049 |
| 50 mM Citrate pH 4 | 0.016 |
| 50 mM Citrate pH 5 | 0.046 |
| 50 mM Citrate pH 6 | 0.044 |
| 50 mM Phosphate pH 7 | 0.049 |
| 50 mM Phosphate pH 8 | 0.043 |
| 50 mM Borate pH 9 | 0.075 |
| 50 mM Borate pH 10 | 0.240 |
| Water | 0.072 |

TABLE 2
Thermodynamic Solubility in Selected Solvents

| | Maximum Compound I concentration |
|---|---|
| Solvent | (mg/mL) |
| Acetone | ≥32 |
| Acetonitrile (ACN) | ≥41 |
| Dichloromethane (DCM) | ≥59 |
| Dimethyl Sulfoxide (DMSO) | ≥22 |
| Ethanol (EtOH) | ≥24 |
| Ethyl Acetate (EA) | ≥48 |
| Isopropyl Alcohol (IPA) | 12.9 |
| Methanol (MeOH) | ≥25 |
| Tetrahydrofuran (THF) | ≥36 |
| 1% Tween 20 | 0.22 |
| 1% Tween 80 | 1.42 |
| 10% HPβCD | ≥5.7 |
| Propylene Glycol | ≥8.9 |
| PEG 400 | ≥10 |
| PEG 300 | ≥9.5 |
| Cottonseed Oil | ≥9.6 |
| Carrier A | 57.1 |

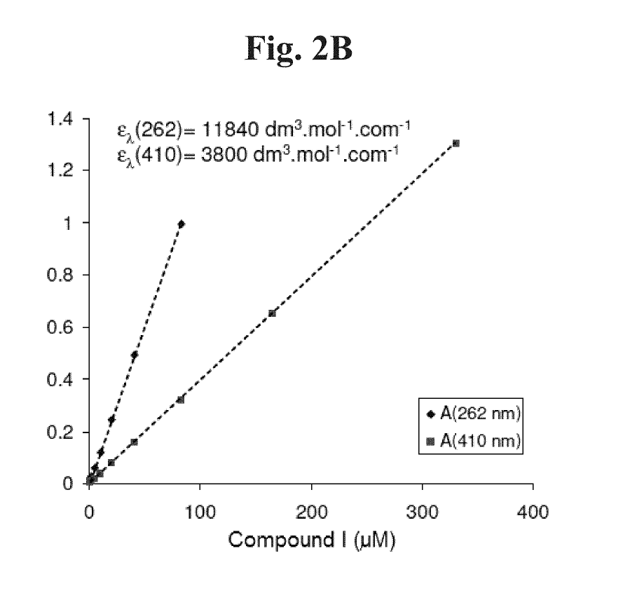

The maximum quantity of Compound I that can be completely dissolved at 25° C. in the several solvents was evaluated using absorption spectrophotometry. Supersaturated dispersions of Compound I in each solvent were incubated at 25° C. for 72 h on a rotary shaker. Samples were then centrifuged at 10,000×g for 10 minutes and the resulting supernatant was filtered through a syringe filter (0.45 um). An aliquot of filtrate was then diluted with methanol or extracted into methanol. The concentration of Compound I was determined using absorption spectrophotometry (DU-640, Beckman Coulter) at 410 nm (e=3,500 dm³ mol⁻¹ cm⁻¹), as shown in FIG. 2. The results are shown in Table 3.

TABLE 3
Solubility of Compound I in Selected Carriers

| | Concentration of | |
|---|---|---|
| | Compound I in | |
| | saturated solution | |
| Carrier | (mg/mL) | ±SD |
| Oils | | |
| TRIACETIN | 200.6 | 1.1 |
| Triglyceride 1,2,3-triacetoxypropane | | |
| TRIBUTYRIN | 156.7 | 0.4 |
| Triglyceride 1,3-D(butanoyloxy) | | |
| propan-2-yl butanoate | | |
| DYNACET ™ 285 | 141.9 | 0.5 |
| acetoglyceride | | |
| LABRASOL ™ | 145.4 | 1.4 |
| Caprylocaproyl polyoxyl-8 glyceride | | |
| LABRAFIL ™ M 1944 CS | 82.1 | 0.5 |
| Oleoyl macrogol-6 glycerides | | |
| CAPMUL ™ PG-12 EP/NF | 83.7 | 0.1 |
| Propylene glycol monolaurate | | |
| CAPMUL ™ MCM EP/NF | 92.0 | 0.6 |
| Glycerol monocaprylocaprate, Type I | | |
| CAPMUL ™ MCM C8 EP/NF | 97.0 | 0.5 |
| Mono/diglycerides of caprylic acid | | |
| CAPTEX ™ 8000 | 107.2 | 0.2 |
| Triglycerides of caprylic acid | | |
| MIGLYOL ™ 840 | 108.6 | 0.5 |
| Propylene glycol dicaprylate/dicaprate | | |
| LABRAFAC ™ LIPOPHILE WL | 104.0 | 0.2 |
| 1349 Medium chain fatty acid | | |
| triglyceride JPE, Carpylic/Capric | | |
| triglyceride (USA FDA IIG) | | |
| CAPRYOL ™ 90 | 110.0 | 0.4 |
| Propylene glycol monocaprylate | | |
| (type II) NF | | |
| PECEOL ™ | 60.5 | 0.1 |
| Glyceryl monooleate, type 40 | | |
| Ethyl oleate | 66.2 | 0.3 |
| Glycerol trioleate | 58.6 | 0.3 |
| Oleic acid | 51.8 | 0.2 |
| Almond oil | 61.7 | 0.0 |
| Canola oil | 58.5 | 0.3 |
| Coconut oil | 97.8 | 0.2 |
| Corn oil | 62.5 | 0.4 |
| Olive oil | 58.1 | 0.3 |
| Palm kernel oil | 82.4 | 0.6 |
| Castor oil | 64.8 | 0.8 |
| Safflower oil | 59.8 | 2.3 |
| Sesame oil | 60.2 | 0.4 |
| Sunflower oil | 58.6 | 0.8 |
| Nonionogenic surfactants | | |
| Sorbitan monooleate, Span 80 | 53.5 | 0.5 |
| Sorbitan trioleate, Span 85 | 57.9 | 0.1 |
| Water-soluble solvents | | |
| Ethanol | 29.5 | 0.1 |
| 2-Propanol | 17.0 | 0.1 |
| 1-Butanol | 24.4 | 0.1 |
| Glycerol | 1.0 | 0.1 |
| N,N-Dimethylacetamide | 520.1 | 1.3 |
| N-Methyl-2-pyrrolidone | 570.6 | 1.3 |
| Propylene glycol | 12.7 | 0.1 |
| Diethylene glycol monoethyl ether | 147.8 | 2.5 |
| Propylene carbonate | 238.4 | 0.4 |

The solubility is indicated as the concentration of Compound I in saturated solution at 25° C.

### Example 2

### Water Uptake of Compound I

For the purposes of establishing material handling practices, Compound I was evaluated for water uptake in various controlled conditions. Compound I was exposed to 25° C./60% relative humidity and 40° C./75% relative humidity for up to one day. Approximately 200 mg of Compound I was accurately weighed into separate glass vials and placed uncapped in stability chambers. At various time points, the vials were removed, stoppered and crimped, and the water content for Compound I powder was determined by standard Karl Fischer coulometric analysis. The results indicate that Compound I appears to be anhydrous. Compound I was not sensitive to water uptake at up to 75% relative humidity. The water content of Compound I at the indicated temperature and RH (relative humidity) are shown in Table 4.

TABLE 4
Water uptake of Compound I

| | Water content | Water Content |
|---|---|---|
| Time point (hours) | 25° C./60% RH | 40° C./75% RH |
| 0 | 0.023% | 0.023% |
| 5 | 0.026% | 0.027% |
| 24 | 0.029% | 0.031% |

### Example 3

### Stability of Aqueous Compound I Formulations

Forced degradation studies were performed to determine the stability of Compound I in various aqueous formulations. Compound I formulations were prepared at approximately 5 mg/g (˜4.6 mg/mL assuming a density of 0.92 g/mL) in various aqueous media, as listed in Tables 5 and 6. The aqueous formulations were placed in stability chambers and subjected to conditions of 25° C./60% relative humidity (RH) and 40° C./75% relative humidity (RH). Samples were pulled from the formulations at 3 and 10 days. At each time point, samples were pulled from the stability chambers, diluted 20× to the nominal concentration using positive displacement pipettes and 50/50 ethanol/hexane as the diluent, and analyzed via HPLC for purity. The purity results are shown in Table 5. The potency results are shown in Table 6. Within the physiological pH range (around pH 7), less than 10% of initial Compound I remained in the solution after 1 week. Compound I was stable in aqueous acid, but not in basic or neutral aqueous conditions.

TABLE 5
Solution Stability in Aqueous Media, Purity (% Area)

| | Condition | | | |
|---|---|---|---|---|
| Sample | (° C./RH) | t = 0 | t = 1 week | t = 2 week |
| pH 1 * | 25/60 | 99.32 | 98.83 | 98.73 |
| | 40/75 | | 98.12 | 95.79 |
| pH 2 * | 25/60 | 98.83 | 98.48 | 96.90 |
| | 40/75 | | 93.43^{‡} | 85.79^{‡} |
| pH 3 * | 25/60 | 98.85 | 97.09 | 95.26 |
| | 40/75 | | 88.81 | 72.78 |
| pH 4 * | 25/60 | 98.73 | 96.77^{‡} | 93.81^{‡} |
| | 40/75 | | 87.32^{‡} | 73.78^{‡} |
| pH 5 * | 25/60 | 98.46 | 92.26^{‡} | 83.09^{‡} |
| | 40/75 | | 58.98^{‡} | 29.30^{‡} |
| pH 6 * | 25/60 | 97.78 | 69.37 | 44.92^{‡} |
| | 40/75 | | <10 | <10^{‡} |
| pH 7 * | 25/60 | 91.09 | <10 | <10^{‡} |
| | 40/75 | | <10 | <10^{‡} |
| pH 8 * | 25/60 | 81.64 | <10 | <10^{‡} |
| | 40/75 | | <10 | <10^{‡} |
| pH 9 * | 25/60 | 89.84 | 30.06 | 7.58^{‡} |
| | 40/75 | | <10 | <10^{‡} |
| pH 10 * | 25/60 | 88.46 | 37.43 | <10^{‡} |
| | 40/75 | | <10^{‡} | <10^{‡} |
| water | 25/60 | 96.33 | 66.97^{‡} | 53.76^{‡} |
| | 40/75 | | 45.09^{‡} | 44.20^{‡} |

*Due to limited solubility the solution stability was evaluated with 50% DMSO.
^{‡}Precipitation was observed. Precipitation may have also occurred in buffered media pH = 6-10 at t = 1 week.
Note:
Purity values <10% were not reported due to poor chromatography.

TABLE 6
Solution Stability in Aqueous Media, % Potency

| | Condition | | | |
|---|---|---|---|---|
| Sample | (° C./RH) | t = 0 | t = 1 week | t = 2 week |
| pH 1 * | 25/60 | 99.62 | 98.65 | 97.75 |
| | 40/75 | | 96.67 | 94.12 |
| pH 2 * | 25/60 | 98.23 | 96.09 | 93.90^{‡} |
| | 40/75 | | 88.34 | 78.74^{‡} |
| pH 3 * | 25/60 | 96.53 | 94.10 | 91.08 |
| | 40/75 | | 83.46 | 69.18 |
| pH 4 * | 25/60 | 96.76 | 91.93^{‡} | 87.26^{‡} |
| | 40/75 | | 75.80^{‡} | 60.57^{‡} |
| pH 5 * | 25/60 | 97.34 | 85.16^{‡} | 73.07^{‡} |
| | 40/75 | | 43.84^{‡} | 19.81^{‡} |
| pH 6 * | 25/60 | 94.27 | 60.26 | 35.92^{‡} |
| | 40/75 | | <10 | <10^{‡} |
| pH 7 * | 25/60 | 87.84 | <10 | <10^{‡} |
| | 40/75 | | <10 | <10^{‡} |
| pH 8 * | 25/60 | 78.28 | <10 | <10^{‡} |
| | 40/75 | | <10 | <10^{‡} |
| pH 9 * | 25/60 | 85.90 | 29.68 | <10^{‡} |
| | 40/75 | | <10 | <10^{‡} |
| pH 10 * | 25/60 | 86.55 | 36.39 | <10^{‡} |
| | 40/75 | | <10^{‡} | <10^{‡} |
| water | 25/60 | 92.86 | 61.83^{‡} | 51.97^{‡} |
| | 40/75 | | 39.96^{‡} | 39.53^{‡} |

^{‡}The purity of many of the aqueous pH stability samples was difficult to assess precipitation.

### Example 4

### Stability of Compound I Formulations in Several Solvents

Forced degradation studies were performed to determine the stability of Compound I in various formulations. Compound I formulations were prepared at approximately 5 mg/g (˜4.6 mg/mL assuming a density of 0.92 g/mL) in various solvents, as listed in Tables 7 thorough 10. The formulations were placed in stability chambers and subjected to conditions of 25° C./60% relative humidity (RH) and 40° C./75% relative humidity (RH). Samples were pulled from the formulations at 3 and 10 days. At each time point, samples were pulled from the stability chambers, diluted 20× to the nominal concentration using positive displacement pipettes and 50/50 ethanol/hexane as the diluent, and analyzed via HPLC for purity. The purity results are shown in Table 7. The potency results are shown in Table 8. Most purity and potency values trended downward throughout the stability study, as shown in Tables 7 and 8. Stability was very good in most organic solvents but not in Tween-20 or Tween-80 surfactant formulations in which 50% or more Compound I was lost. The formulations of Compound I in PEG were 50% stable at 40° C./75%, whereas its stability in Carrier A was excellent. Indeed, as shown in Tables 9 and 10, no significant degradation was observed in the Carrier A formulations after up to 10 days of storage at any of the temperatures and relative humidity conditions tested.

TABLE 7
Solution Stability in Selected Solvents, Purity (% Area)

| | Condition | | | |
|---|---|---|---|---|
| Sample | (° C./RH) | t = 0 | t = 1 week | t = 2 week |
| Acetone | 25/60 | 94.35 | 93.49 | 92.27 |
| | 40/75 | | 91.94 | 91.52 |
| ACN | 25/60 | 95.56 | 93.35 | 91.03 |
| | 40/75 | | 93.33 | 93.59 |
| DCM | 25/60 | 92.84 | 93.85 | 92.99 |
| | 40/75 | | 91.88 | 81.23 |
| DMSO | 25/60 | 94.08 | 93.47 | 92.47 |
| | 40/75 | | 89.28 | 89.26 |
| Diluent | 25/60 | 99.77 | 99.76 | 99.79 |
| | 40/75 | | 99.81 | 99.82 |
| EtOH | 25/60 | 99.37 | 96.08 | 92.82 |
| | 40/75 | | 91.99 | 89.39 |
| EA* | 25/60 | 80.16 | 77.38 | 76.03 |
| | 40/75 | | 77.28 | 77.13 |
| IPA | 25/60 | 98.80 | 95.62 | 93.35 |
| | 40/75 | | 91.10 | 89.40 |
| MeOH | 25/60 | 99.32 | 95.54 | 88.79 |
| | 40/75 | | 86.56 | 83.85 |
| THF | 25/60 | 96.45 | 94.19 | 91.24 |
| | 40/75 | | 92.54 | 89.34 |
| 1% TW20 | 25/60 | 97.00 | 64.23^{‡} | 50.47^{‡} |
| | 40/75 | | 47.19^{‡} | 41.25^{‡} |
| 1% TW80 | 25/60 | 95.11 | 57.32^{‡} | 47.62^{‡} |
| | 40/75 | | 42.07^{‡} | 40.53^{‡} |
| 10% HPβCD | 25/60 | 99.49 | 97.41 | 94.73 |
| | 40/75 | | 91.22 | 87.69 |
| PG | 25/60 | 99.65 | 99.14 | 97.81 |
| | 40/75 | | 97.49 | 96.43 |
| PEG 300 | 25/60 | 98.52 | 88.59 | 80.73 |
| | 40/75 | | 65.16 | 49.31 |
| PEG 400 | 25/60 | 98.33 | 88.42 | 81.00 |
| | 40/75 | | 70.08 | 49.07 |

^{‡}Precipitation was observed.
*poor chromatography observed with ethyl acetate.

TABLE 8
Solution Stability in Selected Solvents, % Potency

| | Condition | | | |
|---|---|---|---|---|
| Sample | (° C./RH) | t = 0 | t = 1 week | t = 2 week |
| Acetone | 25/60 | 89.48 | 88.45 | 86.68 |
| | 40/75 | | 85.86 | 85.36 |
| ACN | 25/60 | 99.87 | 97.47 | 95.33 |
| | 40/75 | | 97.21 | 96.90 |
| DCM | 25/60 | 107.91 | 109.04 | 109.71 |
| | 40/75 | | 109.10 | 94.64 |
| DMSO | 25/60 | 92.72 | 92.23 | 89.41 |
| | 40/75 | | 87.01 | 84.00 |
| Diluent | 25/60 | 100.55 | 101.36 | 99.80 |
| | 40/75 | | 99.92 | 99.87 |
| EtOH | 25/60 | 100.73 | 97.06 | 93.35 |
| | 40/75 | | 92.17 | 89.76 |
| EA* | 25/60 | 85.04 | 83.86 | 80.13 |
| | 40/75 | | 79.91 | 82.73 |
| IPA | 25/60 | 105.30 | 100.58 | 98.70 |
| | 40/75 | | 100.40 | 96.09 |
| MeOH | 25/60 | 103.09 | 98.06 | 92.24 |
| | 40/75 | | 89.40 | 86.05 |
| THF | 25/60 | 106.38 | 104.08 | 101.29 |
| | 40/75 | | 102.15 | 99.82 |
| 1% TW20 | 25/60 | 95.60 | 57.81^{‡} | 46.58^{‡} |
| | 40/75 | | 42.22^{‡} | 37.25^{‡} |
| 1% TW80 | 25/60 | 93.08 | 51.28^{‡} | 42.01^{‡} |
| | 40/75 | | 34.95^{‡} | 32.58^{‡} |
| 10% HPβCD | 25/60 | 97.29 | 94.06 | 91.09 |
| | 40/75 | | 84.86 | 79.52 |
| PG | 25/60 | 109.71 | 107.87 | 106.46 |
| | 40/75 | | 105.69 | 103.92 |
| PEG 300 | 25/60 | 112.73 | 98.16 | 88.15 |
| | 40/75 | | 67.12 | 48.21 |
| PEG 400 | 25/60 | 116.53 | 101.52 | 92.59 |
| | 40/75 | | 73.11 | 54.66 |

^{‡}Precipitation was observed.
*Poor chromatography observed with ethyl acetate. The variation in initial potency values were likely due to sample preparation or solvent effects on the chromatography when used as a diluent.

TABLE 9
Stability of the formulation in Carrier A (sesame oil), Purity as Area - %

| Sample | Condition (° C./RH) | t = 0 (*) | t = 3 day | t = 10 day |
|---|---|---|---|---|
| Carrier A (NF) | 25/60 | 99.7 | 99.2 | 99.4 |
| | 40/75 | | 98.9 | 99.2 |
| Carrier A (SR) | 25/60 | 99.7 | 99.5 | 99.5 |
| | 40/75 | | 99.3 | 99.3 |

(*)The t = 0 purity (area-%) is reported from a representative standard.
NF: laboratory grade/
SR: super refined grade

TABLE 10
Stability of the formulation in Carrier A (sesame oil), Potency as
Amount (mg/mL)

| Sample | Condition (° C./RH) | t = 3 day | t = 10 day |
|---|---|---|---|
| Carrier A (NF) | 25/60 | 4.2 mg/mL | 4.6 mg/mL |
| | 40/75 | 4.2 mg/mL | 4.5 mg/mL |
| Carrier A (SR) | 25/60 | 4.2 mg/mL | 4.8 mg/mL |
| | 40/75 | 4.2 mg/mL | 4.8 mg/mL |

NF: laboratory grade/
SR: super refined grade

### Example 5

### Efficacy of Compound I Oral Formulation Versus Intra-Peritoneal Injection Formulation

The efficacy of the formulation of Compound I in Carrier A for oral administration was compared to the previous formulation of Compound I in poly-ethylene glycol (PEG) for intra-peritoneal injection (i.p.). These experiments were performed using Intra Vital Microscopy (IVM) in a pseudo-orthotopic prostate chamber model in which minced prostate tissue is grafted in chambers surgically placed in the dorsal skinfold of mice, and allowed to vascularize. Small tumor cells spheroids were then placed on the prostate tissue, creating a biologically relevant prostatic environment. The tumor cells that express fluorescent H2B-GFP and can be detected in situ and in real-time through the microscopy window of the dorsal chamber.

Epithelial cell lines derived from the prostate tumor of a PTEN-null mouse were used. Mouse prostate cancer cells PTEN-P2 are heterozygous for PTEN deletion and do express protein PTEN. They are also androgen receptor (AR) positive and androgen-dependent for growth.

Titanium chambers were placed by surgery in nude mice and syngeneic prostate tissue was grafted into the chamber two days later. Tumor spheroids containing 70,000 mouse prostate cancer cells PTEN-P2/H2B-GFP were placed on the prostate tissue 7 days later. In this particular experiment all the mice were castrated 21 days later and before initiation of Compound I treatment to investigate the combination of castration with Compound I. Compound I, which was previously shown to synergize with castration in mice, was administered orally at various doses in a Carrier A formulation, or via intra-peritoneal injection at 1 mg/kg/day in DMSO/PEG formulation. Changes in tumor size were monitored by IVM and quantified. Results are expressed as tumor size relative to their size at the start of treatment and as a function of time.

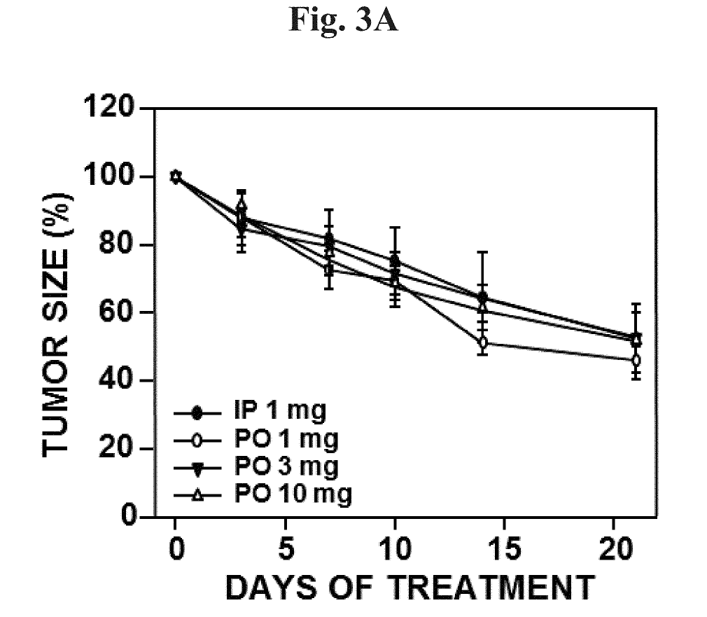

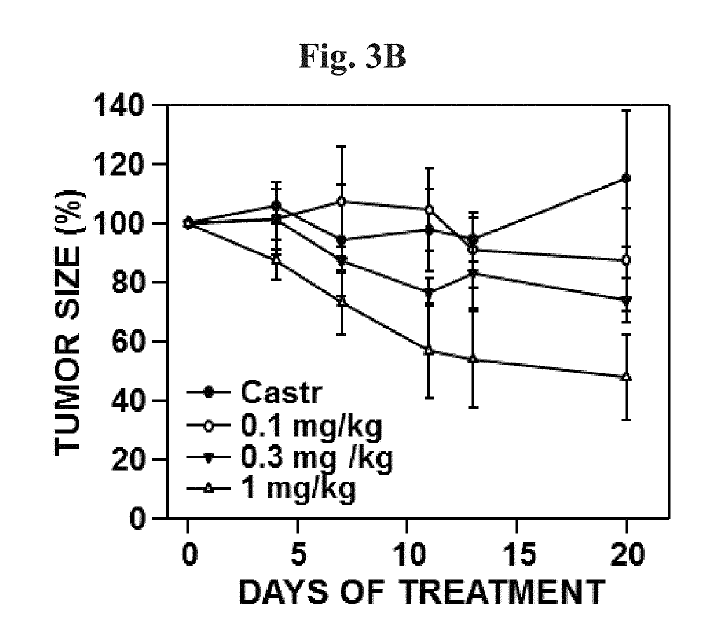

As shown in FIG. 3A, Compound I in PEG decreased tumor sizes by 50% (p<0.001). The oral formulation of Compound I at 1 mg/kg/day in Carrier A caused tumor regression as effectively as i.p. injection of 1 mg/kg Compound I in PEG. Higher doses of Compound I of 3 and 10 mg/kg/day in oil showed no improvement over the dose of 1 mg/kg/day. On the other hand, lower doses of Compound I were less efficient at causing tumor regression (FIG. 3B). The oral administration of Compound I was well tolerated since high doses of up to 10 mg/kg/day for 21 days could be used without eliciting major side effects, based on lack of changes in whole body weights or in animal behavior. The oil formulation was surprisingly found to be as efficient as the previous PEG formulation administered i.p., with an optimal effect at the dose of 1 mg/kg/day. Based on these results, the formulation of Compound I in Carrier A was selected for use in the clinic.

### Example 6

### Toxicology of Oral Formulation of Compound I

A single-dose maximum tolerated dose (MTD) study and a seven consecutive day dose-range finding study were carried out in Sprague Dawley by orally administering a formulation of Compound I in Carrier A. In the MTD phase, doses of 50, 100, 200 and 300 mg/kg were administered once to Sprague Dawley rats by oral gavage. The single-dose MTD of Compound I in rats was found at 100 mg/kg. Acute administration of Compound I at 200 or 300 mg/kg resulted in unscheduled deaths in male rats on Days 1 and 2 but not in females. At these high doses, the rats exhibited adverse clinical events such as decreased activity, abnormal body carriage and piloerection. Male rats also exhibited impaired righting reflex and slow or shallow breathing.

Daily doses of 0 (vehicle), 10, 30 and 100 mg/kg were then administered to Sprague Dawley rats by oral gavage for seven days for the dose-range finding study. Morbidity/mortality occurred both in male and female rats at 100 mg/kg/day beginning on day 3, resulting in early termination of all animals on Day 5. Adverse events included decreased body weight or decreased body weight gain, decreased food consumption, adverse effects on various pathological parameters and Compound I-related macroscopic and microscopic abnormalities in multiple organs. At the 30 mg/kg/day mid-dose level, minimal increases in mean serum total bilirubin concentration and alkaline phosphatase activity were observed. Minimal to mild non-adverse microscopic abnormalities in various organs, indicative of inflammation, were noted in both sexes. Compound I-related effects at the 10 mg/kg/day low-dose level were limited to minimal changes noted in the teeth of one male. The toxicity profile of Compound I is encouraging considering that the drug is effective in rodents at much lower doses. This toxicity-to-effectiveness dose ratio provides, potentially, a wide therapeutic window.

### Example 7

### Effectiveness of Oral Formulation of Compound I in Combination with Castration

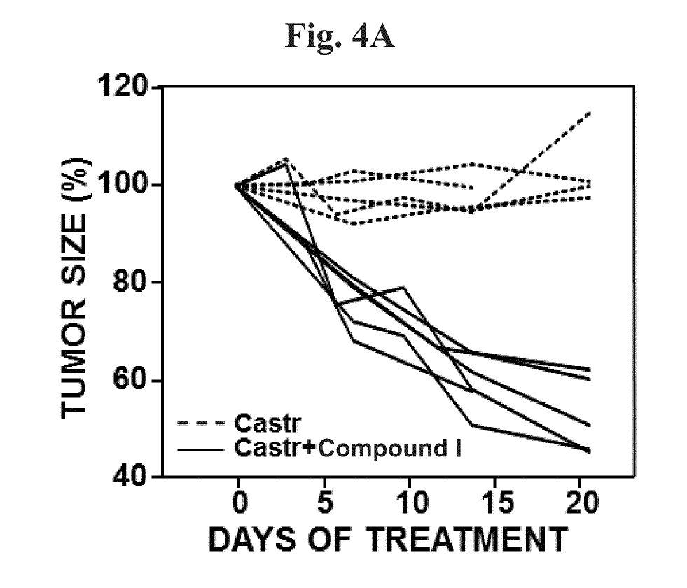

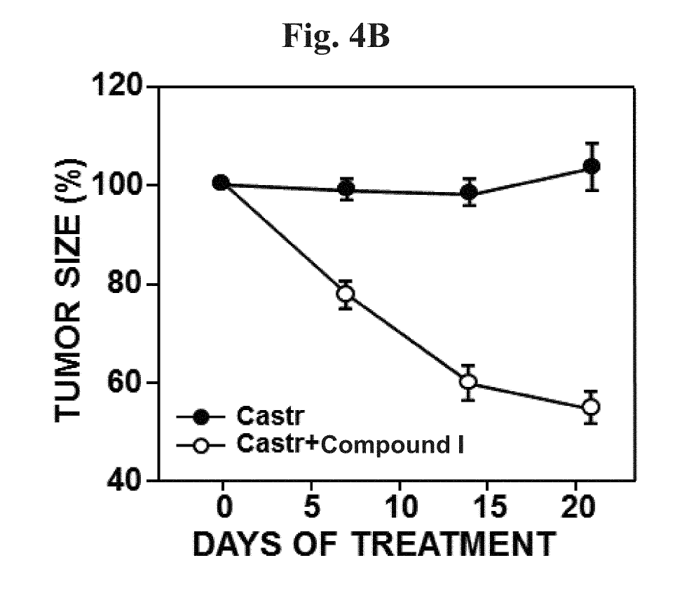

Clinical trials in cancer research are performed against the current standard treatment, which for prostate cancer is androgen deprivation therapy (ADT). Surgical castration in the mouse effectively depletes circulating androgens and is accepted in the art as an appropriate model for ADT in humans. Therefore, the investigative treatment of castration combined with Compound I was compared to standard treatment with surgical castration alone. Six cohorts of mice totaling 52 mice were castrated and treated or not with Compound I. Some of these cohorts were used as controls for other investigative treatment arms. Comparing the trend lines of several separate cohorts, as shown in FIG. 4A, indicated that results were consistent across cohorts and over time, with 40 to 50% tumor regression observed after 21 days of combination treatment in all experiments. FIG. 4B represents the compilation of all animals for which data was available at the indicated time points. The difference between castration alone and the combination arm was statistically significant (p<0.001, two-way repeated measures ANOVA with Bonferroni post hoc).

### Example 8

### Oral Formulation of Compound I in Combination with Castration Increases Survival

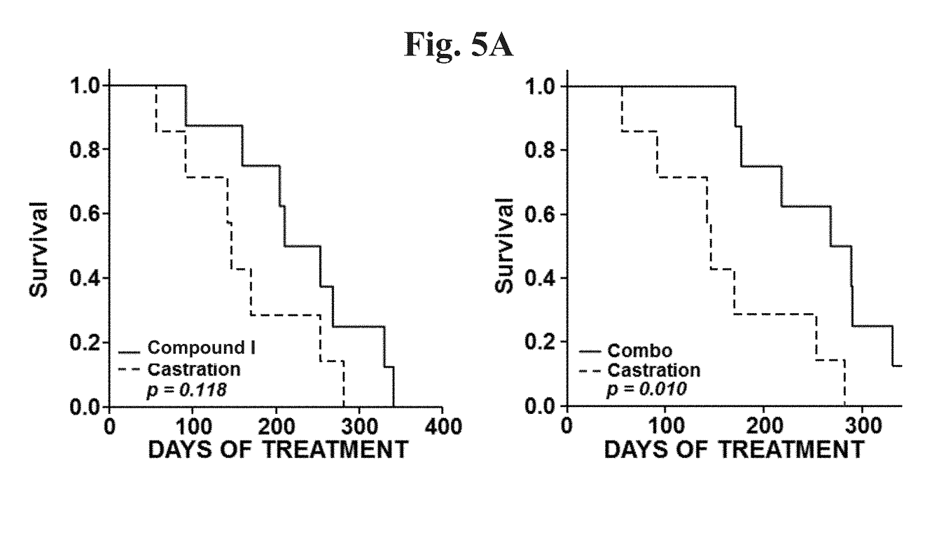

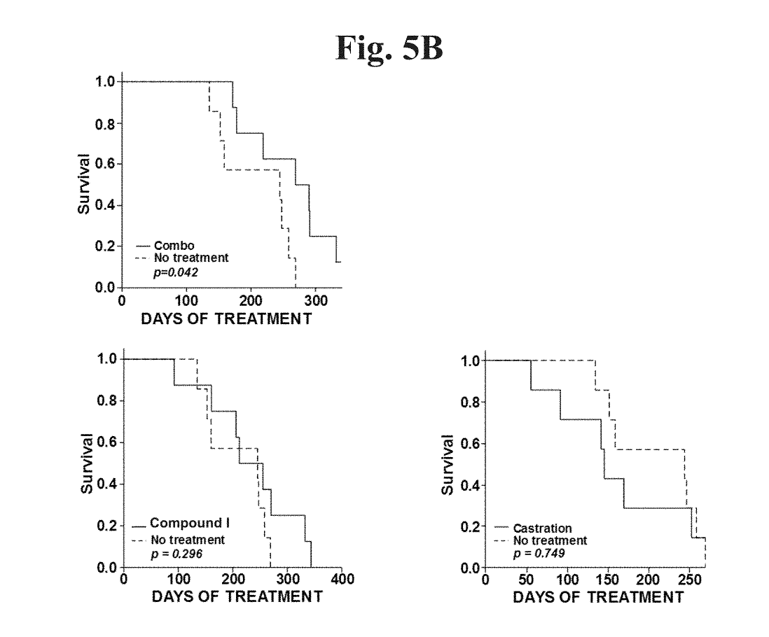

An orthotopic model of prostate cancer in which 30,000 PTEN-P2 mouse cells were injected into the prostate of Fox1/Nu mice was used. Mice were castrated 5 weeks after the PTEN-P2 mouse cells were injected into the prostate, which allowed the tumors to become established. Oral administration of Compound I in Carrier A was initiated one week later at a dose of 1 mg/kg/day. The number of days mice lived following treatment initiation was recorded for each animal. As shown on the right-hand side of FIG. 5A, mice treated with the combination of the oral formulation of Compound I and castration (solid line) survived significantly longer than mice treated with castration alone (dashed line) (p=0.01). As shown on the left-hand side of FIG. 5A, the difference between treatment with Compound I alone (solid line) and castration alone (dashed line) was not statistically different (p=0.118). Of note, two of the mice in the combination treatment group died without signs of cancer, as post-mortem autopsy revealed that neither animal carried prostate tumors or metastases.

As shown in the top plot in FIG. 5B, mice subjected to the combination treatment of the oral formulation of Compound I and castration (solid line) survived longer than untreated control mice (dashed line) and was statistically significant (p=0.042). The bottom-left plot in FIG. 5B shows that the difference between treatment with Compound I alone (solid line) as compared to untreated control (dashed line) was not statistically different (p=0.296). The bottom-right plot shows that the difference between treatment with castration alone (solid line) as compared to untreated control (dashed line) was not statistically different (p=0.749) and did not improve survival. Although treatment with castration alone initially impeded tumor growth, tumors rapidly overcame the lack of hormone and grew more aggressively.

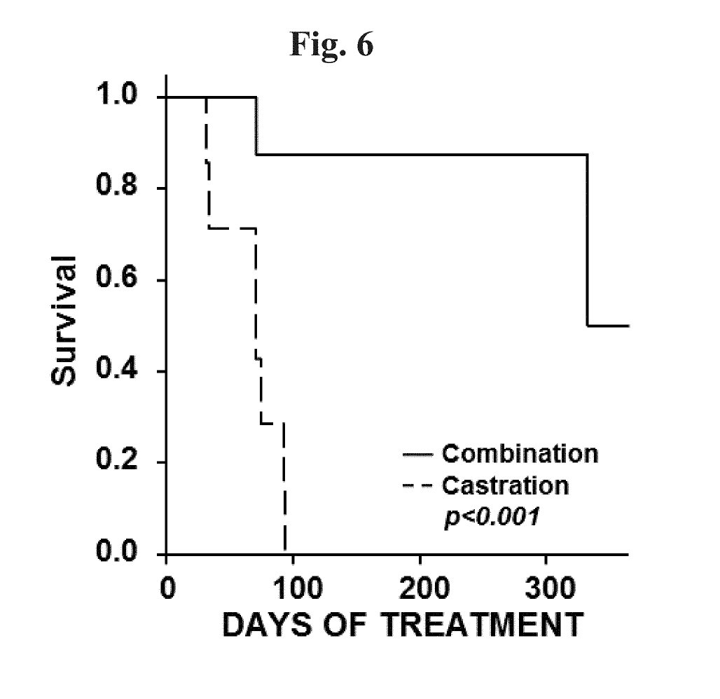

A second study, shown in FIG. 6, used cells derived from the transgenic adenocarcinoma of the mouse prostate (TRAMP) model. The TRAMP model was generated through prostate-specific expression of the large T antigen of SV40 in the prostate epithelium. Male TRAMP mice usually develop epithelial hyperplasia by 8 weeks of age that progresses to adenocarcinomas with a penetrance of 100%. TRAMP-C2 prostate cancer cell lines were derived from the TRAMP mouse. Thirty-thousand (30,000) TRAMP-C2 tumor cells were injected into the prostate of C57BL/6 mice. Five weeks later all the mice were castrated. Mice were treated or not with Compound I at a dose of 1 mg/kg per oral. In this model, 50% of the combination-treated mice survived 200 days longer than mice that had been treated by castration alone. The difference between castration alone and the combination treatment was statistically significant (p<0.001).

These results surprisingly demonstrate that the combination of an orally administered formulation of Compound I in Carrier A with castration not only causes tumor regression but also increases the survival of model mice compared to castration alone.

### Example 9

### Oral Formulation of Compound I in Combination with Chemical ADT

The effect of an oral formulation of Compound I in combination with various drugs that chemically induce androgen ablation was assessed. Thus, the effect of an oral formulation of Compound I in combination with androgen deprivation therapy (ADT) was assessed.

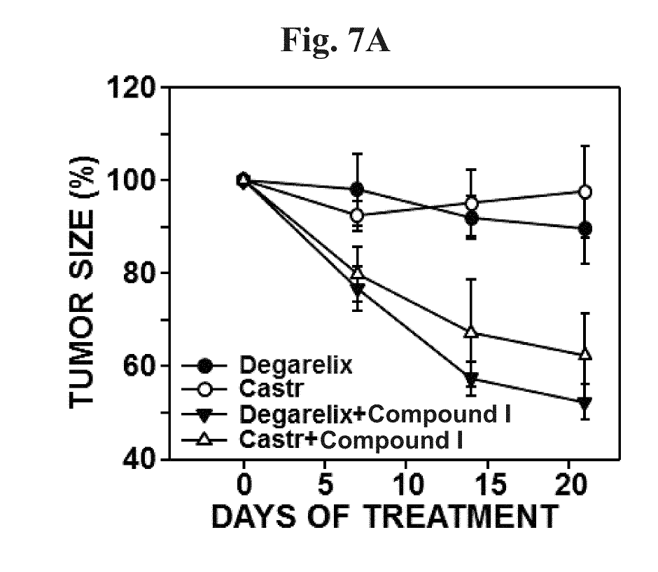

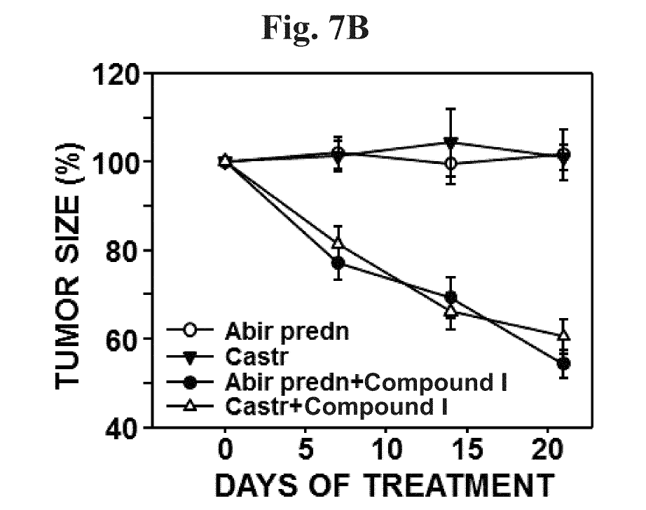

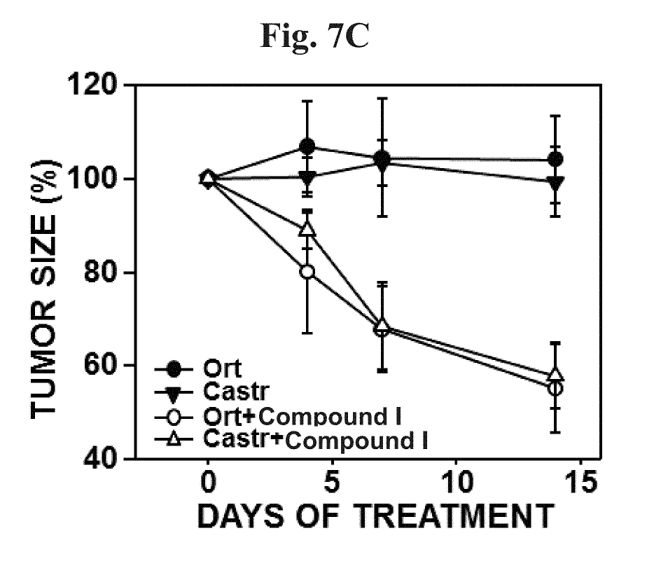

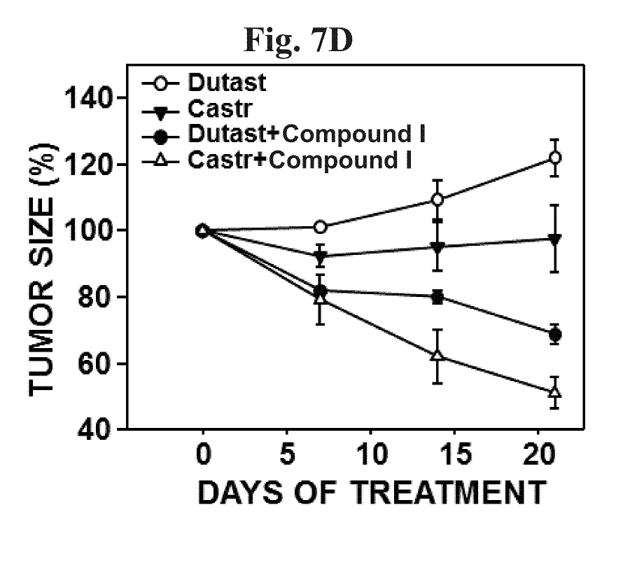

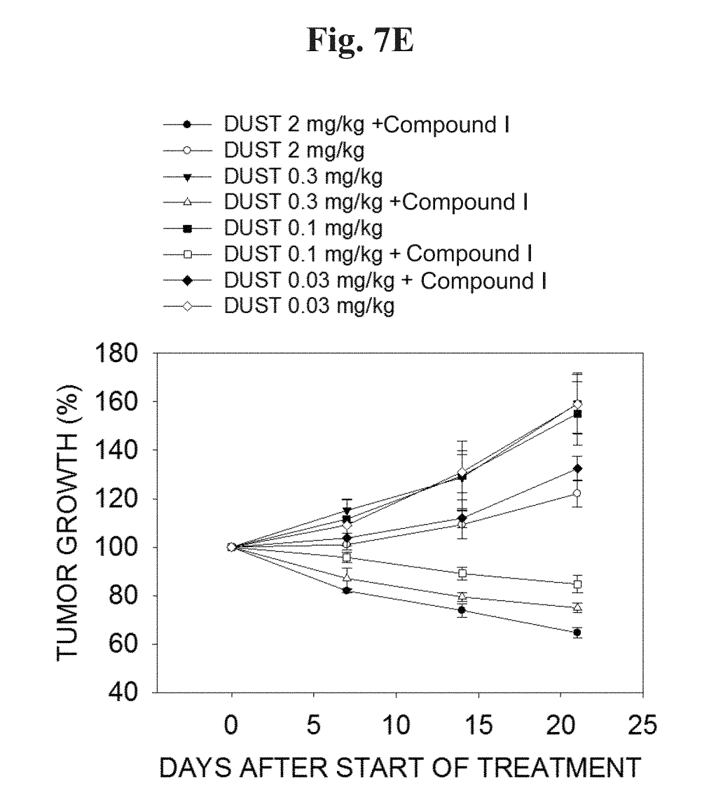

These experiments were performed using IVM in the pseudo-orthotopic prostate chamber model with PTEN-P2/H2B-GFP cells as described above. Oral administration of Compound I in Carrier A was initiated at a dose of 1 mg/kg/day. Chemical ADT was compared to surgical castration to control for the efficacy of chemical ADT at stopping tumor growth, and an oral formulation of Compound I was combined with either chemical ADT or surgical castration. The results are shown in FIG. 7.

FIG. 7A shows the in vivo effects of degarelix alone (closed circles), castration alone (open circles), administering a formulation of Compound I in Carrier A orally in combination with degarelix (closed triangles), and administering a formulation of Compound I in Carrier A orally in combination with castration (open triangles) on tumor size. Chemical castration using degarelix for ADT therapy alone resulted in a minor reduction in tumor size. Surgical castration alone did not result in a reduction in tumor size. The combination of administering a formulation of Compound I in Carrier A orally with degarelix induced an approximately 50% shrinkage of the tumors. The combination of the oral formulation of Compound I with degarelix induced more shrinkage of tumors than the combination of Compound I and castration. The combination of the oral formulation of Compound I with degarelix was more efficient than using degarelix for chemical ADT by itself.

FIG. 7B shows the in vivo effects of abiraterone/prednisone alone (open circles), castration alone (closed triangles), administering a formulation of Compound I in Carrier A orally in combination with abiraterone/prednisone (closed circles), and administering a formulation of Compound I in Carrier A orally in combination with castration (open triangles) on tumor size. Chemical castration using abiraterone/prednisone for ADT therapy alone did not result in a reduction in tumor size. Surgical castration alone did not result in a reduction in tumor size. The combination of administering a formulation of Compound I in Carrier A orally with abiraterone/prednisone induced shrinkage of the tumor size to less than 60% of the starting size. The combination of the oral formulation of Compound I with abiraterone/prednisone induced more shrinkage of tumors than the combination of Compound I and abiraterone/prednisone. The combination of the oral formulation of Compound I with abiraterone/prednisone was more efficient than using abiraterone/prednisone for chemical ADT by itself.

FIG. 7C shows the in vivo effects of orteronel alone (closed circles), castration alone (close triangles), administering a formulation of Compound I in Carrier A orally in combination with orteronel (open circles), and administering a formulation of Compound I in Carrier A orally in combination with castration (open triangles) on tumor size. Chemical castration using orteronel for ADT therapy alone did not result in a reduction in tumor size. Surgical castration alone did not result in a reduction in tumor size. The combination of administering a formulation of Compound I in Carrier A orally with orteronel induced shrinkage of the tumor size to less than 60% of the starting size. The combination of the oral formulation of Compound I with orteronel induced more shrinkage of tumor size than the combination of Compound I and castration. The combination of the oral formulation of Compound I with orteronel was more efficient than using orteronel for chemical ADT by itself.

FIG. 7D shows the in vivo effects of dutasteride alone (open circles), castration alone (close triangles), administering a formulation of Compound I in Carrier A orally in combination with dutasteride (closed circles), and administering a formulation of Compound I in Carrier A orally in combination with castration (open triangles) on tumor size. Chemical castration using dutasteride for ADT therapy alone did not result in a reduction in tumor size, since tumors grew to 120% of their initial size. Surgical castration alone did not result in a reduction in tumor size. The combination of administering a formulation of Compound I in Carrier A orally with dutasteride induced shrinkage of the tumor size by approximately 30%. The combination of the oral formulation of Compound I with dutasteride was more efficient than using dutasteride for chemical ADT by itself. Dutasteride (AVODART®) is used in combination with tamsulosin in the treatment of benign prostatic hyperplasia or to delay cancer progression in men with low-risk prostate cancer. The combination of Compound I with dutasteride induced a tumor regression that improved the effect of dutasteride (p<0.001).

FIG. 7E shows the in vivo effects of administering dutasteride alone as compared to a formulation of Compound I in Carrier A orally in combination with dutasteride on tumor size using several different dosages of dutasteride. Dutasteride was administered alone at 2 mg/kg (open circles), 0.3 mg/kg (closed triangles), 0.1 mg/kg (closed squares), and 0.03 mg/kg (open diamonds). Compound I in Carrier A was administered orally in combination with dutasteride at 2 mg/kg (closed circles), 0.3 mg/kg (open triangles), 0.1 mg/kg (open squares), and 0.03 mg/kg (closed diamonds). The combination of Compound I with dutasteride caused tumor regression proportionally to the dose of dutasteride. At the highest dose of 2 mg/kg dutasteride (closed circles), which is higher than the recommended clinical dose of 0.5 mg/capsule/day, the combination of the oral formulation of Compound I with dutasteride was nearly as efficient as the combination of Compound I with surgical castration. This raises the possibility that an oral formulation of Compound I may help stabilize patients with low risk prostate cancer or with prostatic intraepithelial neoplasia (PIN), and that the combination of Compound I with doses of dutasteride comparable or marginally higher than the current recommended dose may provide clinical benefits.

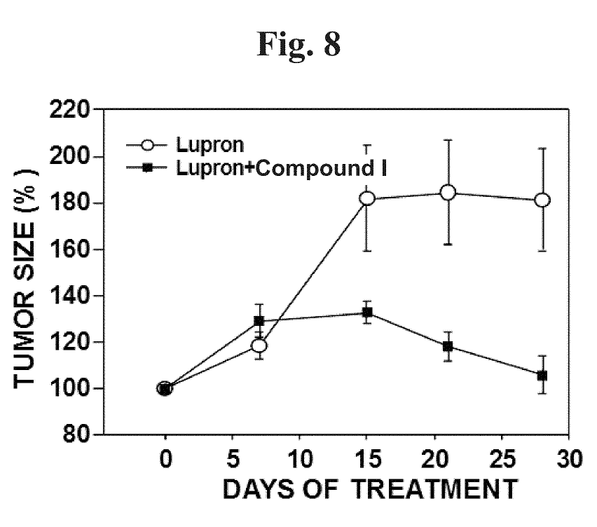

FIG. 8 shows the in vivo effects of leuprolide alone (open circles) as compared to administering a formulation of Compound I in Carrier A orally in combination with leuprolide (closed squares) on tumor size. All mice were administered a one-time intramuscular injection of Lupron depot 3.75 mg (leuprolide acetate for injection) at a dose of 0.187 mg/mouse, and were separated in two treatment groups, with or without Compound I administered orally at a dose of 1 mg/kg/day. Tumor sizes were measured by IVM at day 7, 14, 21 and 28 after start of treatment. Results are expressed as percent of initial tumor size (set at 100%) and represent means±SE (Lupron only n=7; combination n=8). The difference between treatments was statistically significant (p=0.014, two-way repeated measures ANOVA). Leuprolide alone stopped tumor growth after a period of latency of 15 days, during which time the tumors almost doubled in size. This tumor flare, which is also observed in the clinic, is due to the fact that leuprolide initially stimulates the hypothalamic-pituitary-gonadal axis, causing a surge in testosterone levels that increases the proliferation of prostate cancer cells. As shown in FIG. 8, the tumor flare was shorter and of lesser amplitude for the combination of the oral formulation of Compound I and leuprolide as compared to treatment with leuprolide alone. Tumors treated with leuprolide reached 180% of their initial size, whereas tumors treated with the combination reached a maximum 120% of initial tumor size. Treatment with leuprolide alone arrested tumor growth after 15 days of treatment but did not decrease tumor sizes. The combination of leuprolide and the oral formulation of Compound I caused the tumors to shrink.

Compound I in Combination with Androgen Receptor Antagonists

Androgen receptor (AR) antagonists may be used as an alternative to chemical ADT. The effect of Compound I combined with the two AR antagonists, bicalutamide (CASODEX®) and enzalutamide (XTANDI®), was assessed.

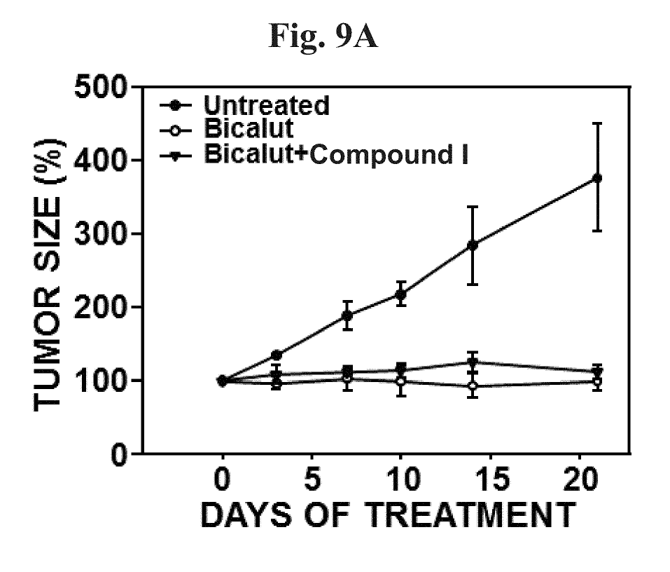

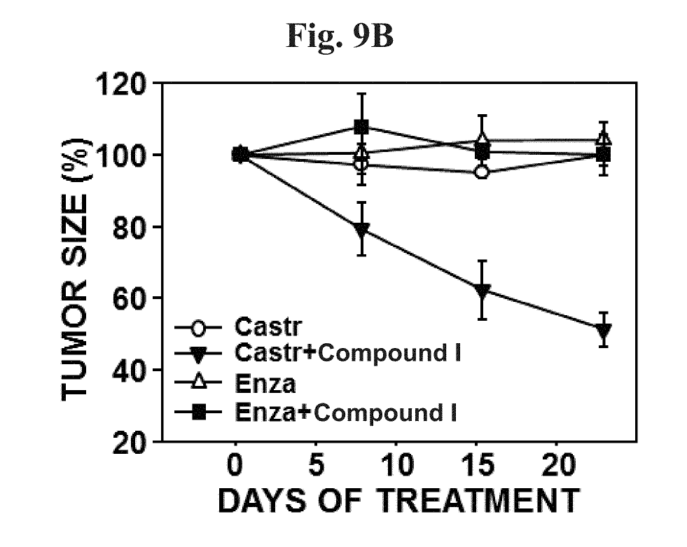

FIG. 9A shows the in vivo effects of no treatment (closed circles), bicalutamide alone (open circles), and administering a formulation of Compound I in Carrier A orally in combination with bicalutamide (closed triangles) on tumor size. No treatment led to significant increase in tumor size. Administration of AR antagonist bicalutamide alone did not result in a reduction of initial tumor size, although it inhibited further tumor growth. The combination of administering a formulation of Compound I in Carrier A orally with bicalutamide did not result in a reduction of initial tumor size. The combination of bicalutamide with Compound I was no more effective than treatment with the drug alone, indicating that Compound I does not improve the efficacy of this AR antagonist.

FIG. 9B shows the in vivo effects of castration alone (open circles), administering a formulation of Compound I in Carrier A orally in combination with castration (closed triangles), enzalutamide alone (open triangles), and administering a formulation of Compound I in Carrier A orally in combination with enzalutamide (closed squares) on tumor size. Castration alone did not result in a reduction in tumor size. Administration of AR antagonist enzalutamide alone did not result in a reduction in tumor size. The combination of administering a formulation of Compound I in Carrier A orally with enzalutamide did not result in a reduction in tumor size. The combination of enzalutamide with Compound I was no more effective than treatment with the drug alone, indicating that Compound I does not improve the efficacy of this AR antagonist.

Summary of Oral Formulation of Compound 1 in Combination with ADT and AR Antagonists

A summary of experimental results on the efficacy of a formulation of Compound I in Carrier A administered orally in combination with surgical castration, leuprolide, leuprolide, degarelix, abiraterone, orteronel, dutasteride, bicalutamide, and enzalutamide for treating prostate cancer is shown in Table 11.

TABLE 11
Summary of the efficacy of Compound I administered orally in
combination with various prostate cancer drugs.

| | | | Combination |
|---|---|---|---|
| Drug type | Drug/method | Mechanism of action | efficacy |
| Decrease | Surgical | Removal of hormone- | Yes |
| DHT | castration | producing tissue | |
| | Leuprolide | Agonist of GnRH | Yes |
| | | receptors | |
| | Degarelix | Antagonist of GnRH | Yes |
| | | receptors | |
| | Abiratcrone | Inhibitor of CYP17A1 | Yes |
| | Orteronel | Inhibitor of CYP17A1 | Yes |
| | Dutasteride | Inhibitor of 5α-reductase | Yes |
| Antagonize | Bicalutamide | Binds to AR and prevents | No |
| AR | | its activation | |
| | Enzalutamide | Binds to AR; prevents | No |
| | | co-activator binding and | |
| | | binding to DNA | |

### Example 10

### Oral Formulation of Compound I in Combination with Chemical ADT for CRPC

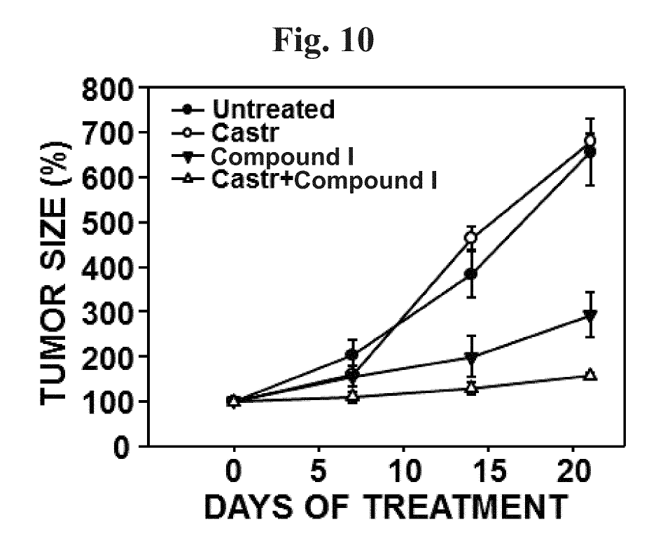

Mouse prostate cancer cells PTEN-CaP2 were initially derived from the PTEN-P2 cell line and are homozygous for PTEN deletion. Complete ablation of PTEN results in intrinsic androgen-independence in the PTEN-null model. Although PTEN-CaP2 tumors express AR, they are resistant to castration in the absence of prior exposure to ADT. This is illustrated in FIG. 10, which shows that PTEN-CaP2/H2B-GFP tumors in castrated mice grew as fast as PTEN-CaP2/H2B-GFP tumors in untreated mice.

FIG. 10 shows the in vivo effects of no treatment (closed circles), castration alone (open circles), administering a formulation of Compound I in Carrier A orally alone (closed triangles), and administering a formulation of Compound I in Carrier A orally in combination with castration (open triangles) on tumor size for castration resistant prostate cancer (CRPC). The oral formulation of Compound I alone inhibited tumor growth, with tumor sizes in Compound I-treated mice reaching 293% of initial size, compared to 680% in castrated mice and 656% in untreated control mice. Even though PTEN-CaP2 are androgen-independent for growth, the combination of Compound I and castration was twice as effective as Compound I alone since tumor growth reached 157% of initial size in the combination group compared to 293% in the combination group (p=0.048). These results indicate that an oral formulation of Compound I may be used in combination with second or third-line ADT therapy to treat CRPC.

### Example 11

### Viscosity of Compound I Formulation

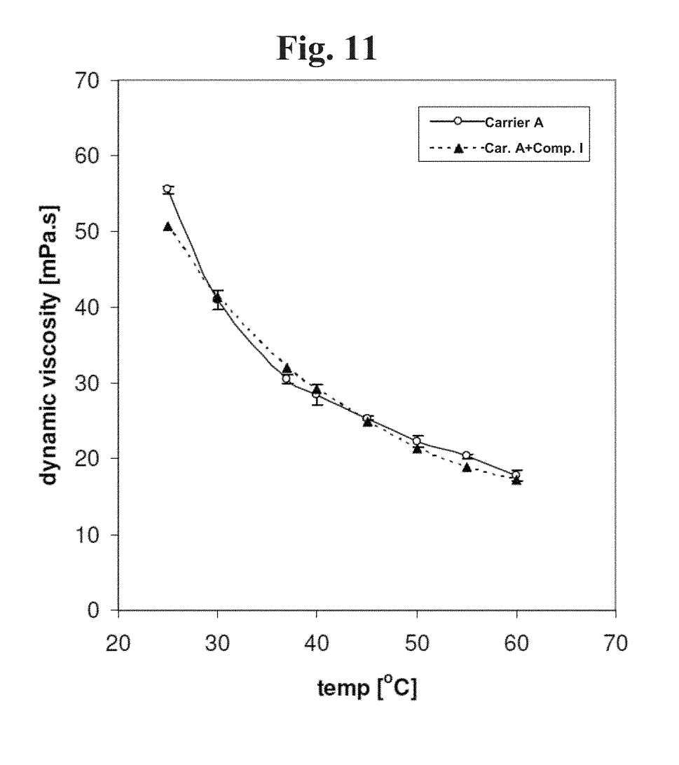

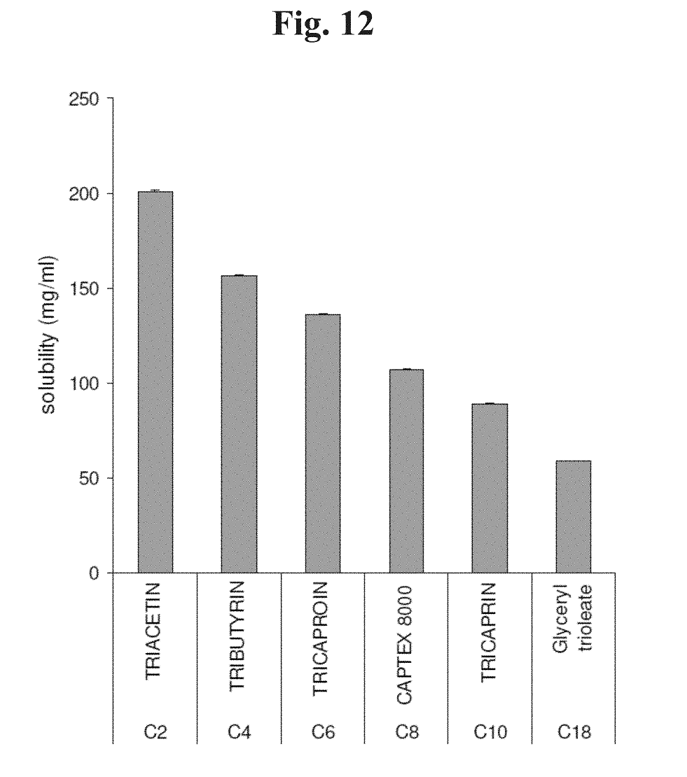

The dynamic (shear) viscosity of pharmaceutically acceptable Carrier A (sesame oil) alone and a pharmaceutical composition containing Compound I and pharmaceutically acceptable Carrier A at a concentration of 50 mg Compound I per 1 mL of Carrier A was determined using a NDJ-5S digital rotary viscometer at various temperatures. The temperature was adjusted using an Isotemp 202 water bath. The results are shown in FIG. 11.

### Example 12

### Self-Nanoemulsifying Formulations of Compound I

The following example demonstrates methods for preparing self-nanoemulsifying formulations comprising Compound I.

Capryol™ 90 (propylene glycol monocaprylate) was selected as an oil phase and Labrasol™/Kolliphor® RH40 (caprylocarpoyl polyoxyl-8 glycerides/polyoxyl castor oil) as a nonionic surfactant/cosurfactant mixture (at a 1:1 ratio) based on pre-formulatory studies of miscibility of various systems of oils and surfactants, including the water solubility and Compound I solubility.

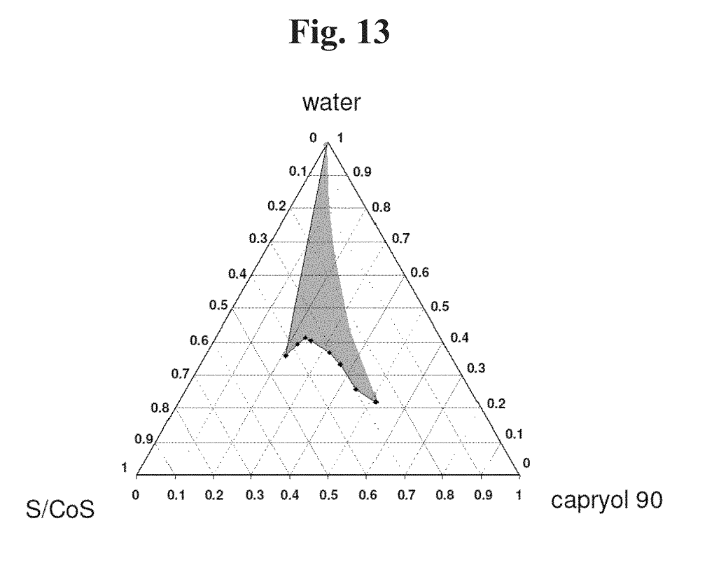

To identify a monophasic optically isotropic nanoemulsion region, a series of mixtures of propylene glycol monocaprylate with surfactant/cosurfuctant Labrasol™/Kolliphor® RH40 (caprylocarpoyl polyoxyl-8 glycerides/polyoxyl castor oil) (1:1, w/w) were prepared. Pseudoternary phase diagrams were constructed using progressive water titration. The established pseudoternary phase diagram shown in FIG. 13 was used to delineate the nanoemulsion domain and boundary of phases.

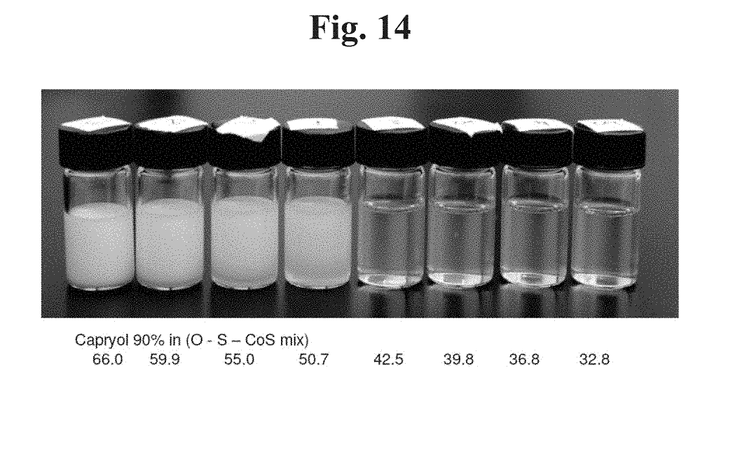

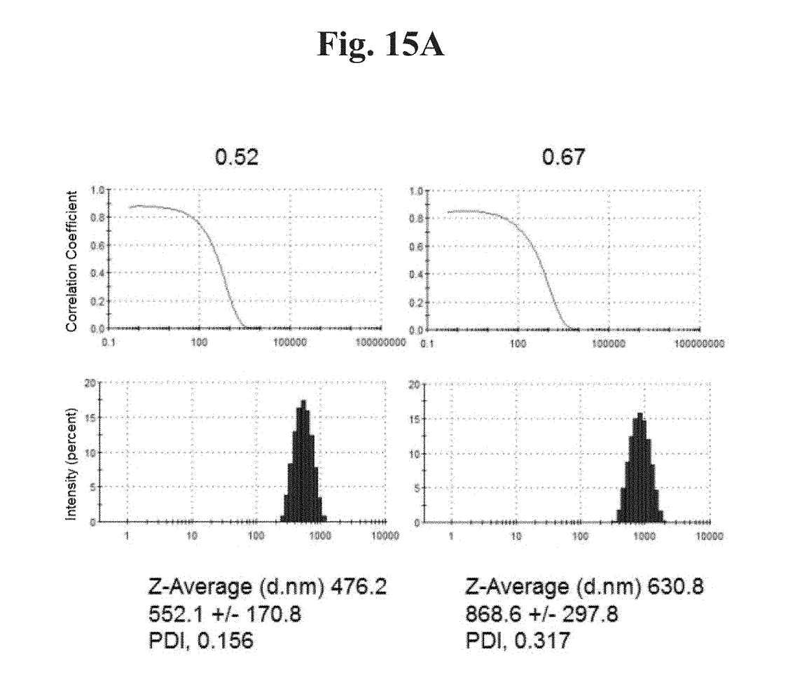

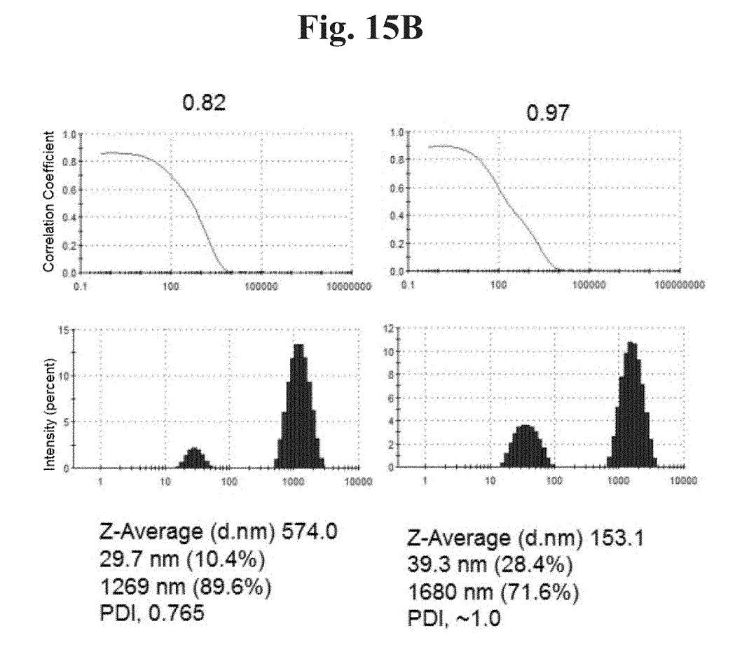

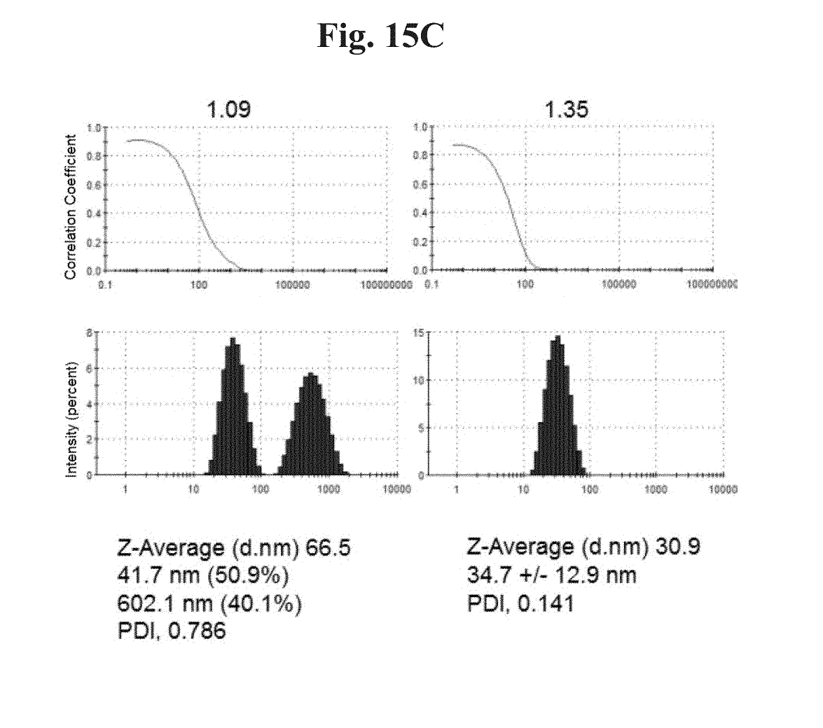

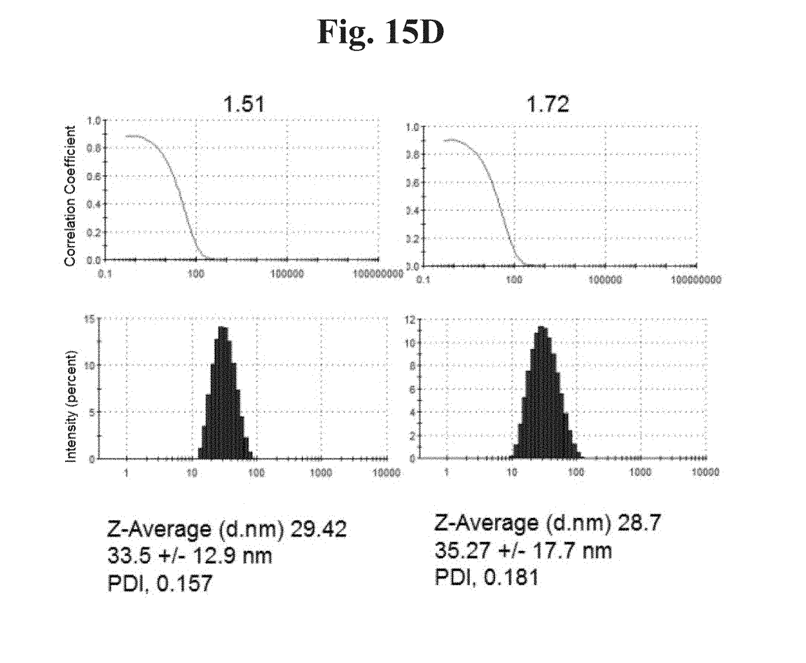

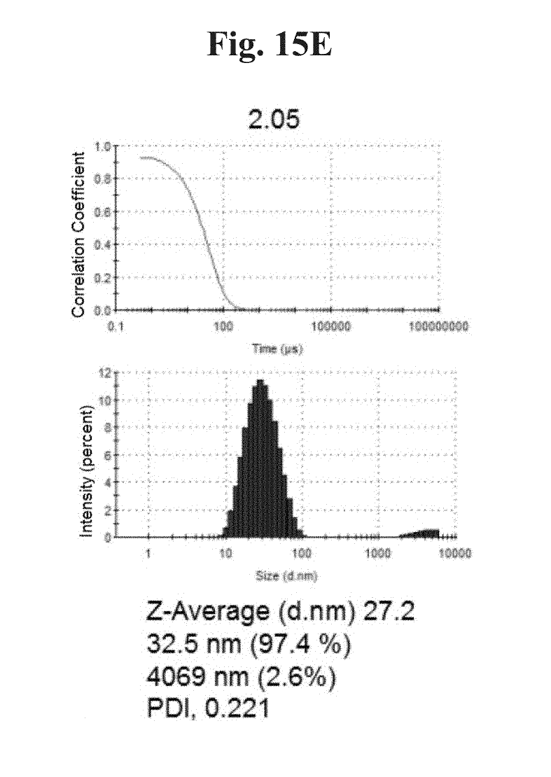

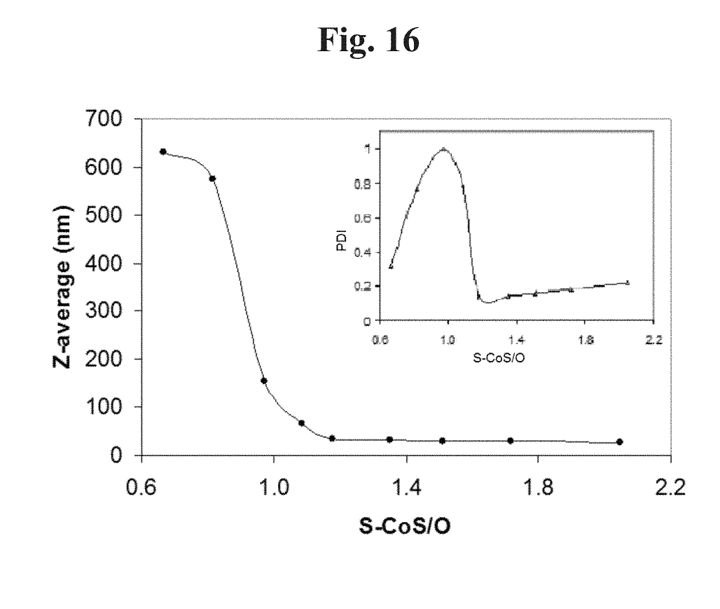

Samples from nanoemulsion area appeared translucent whereas mixtures with a higher percentage of propylene glycol monocaprylate oil phase led to formation of opalescent coarse dispersions (FIG. 14). Aqueous dispersions of samples with different surfactant/cosurfactant to oil ratio were further characterized by photon correlation microscopy (PCS) using Zetasizer Nano-ZS (Malvern). PCS, dynamic light scattering-based at 173° backscattering angle was used to estimate particle size distribution and polydispersity of population (FIGS. 15A-15E). Z-average, an intensity based harmonic mean determined by method of cumulants that showed dependence on surfactant/cosurfactant to oil ratio (FIG. 16). The size of hydrodynamic diameter fell into the range of nanoemulsions at 5-CoS/O>1.2 with a narrow polydisperity index (PDI) (FIG. 16, inset).

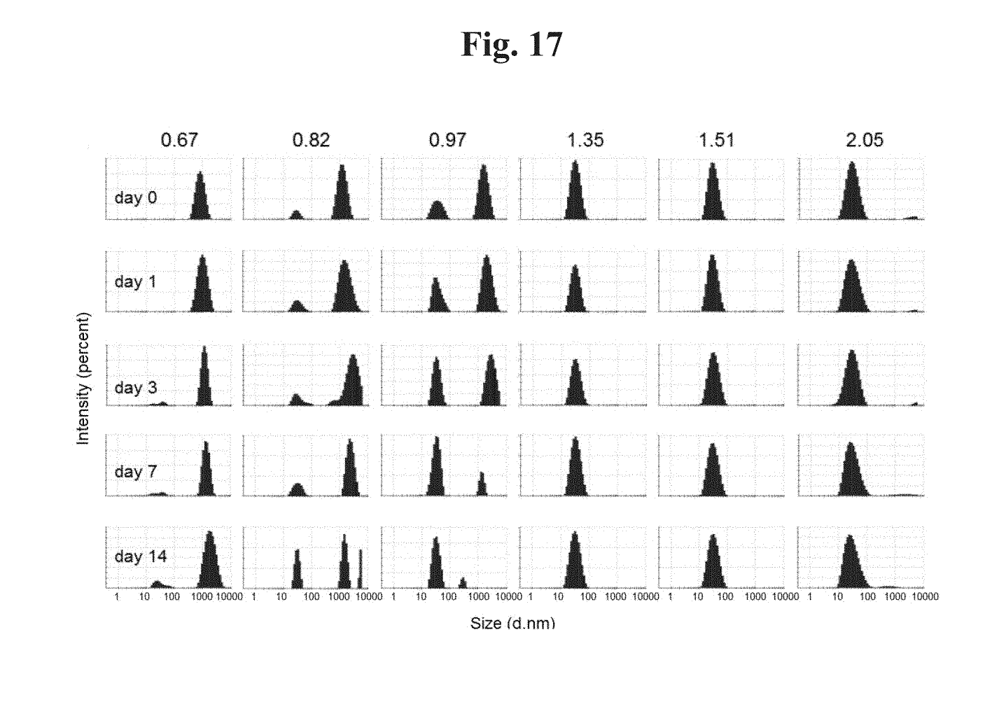

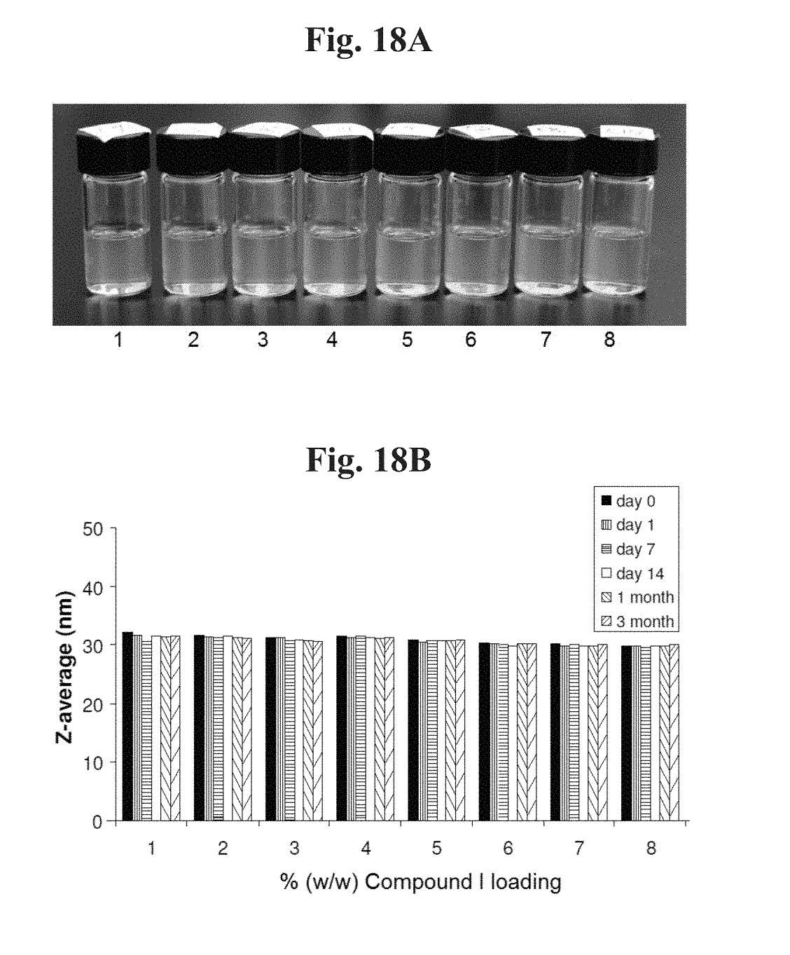

Analysis of particle size distribution over time showed a polydisperse profile and instability of systems with S-CoS/O<1, while systems with S-CoS/O at 1.35 showed narrow PDI and stability of size distribution over time (FIG. 17). A nanoemulsion system with a S-CoS/O ratio of 1.35 was loaded with different amounts of Compound I and nanoemulsions showed stable size distribution profile over time (FIGS. 18A and 18B). Furthermore, both control nanoemulsions (empty nanoemulsions) and nanoemulsions loaded with Compound I appeared to have stable size distribution in different solutions simulating physiological fluids (Table 12).

TABLE 12
Z-average hydrodynamic radius (nm) and PDI in various solutions.

| | | | 0.1M NaH₂PO₄/Na₂HPO₄ | |
|---|---|---|---|---|
| | water | HCl 0.1M | pH 6.8 | pH 7.5 |
| Empty (C0) | 32.2 (0.180) | 30.6 (0.098) | 32.4 (0.173) | 33.8 (0.165) |
| C10: | 30.0 (0.162) | 30.1 (0.135) | 30.0 (0.142) | 31.74 (0.135) |
| Compound I | | | | |
| loaded | | | | |
| (4.7% w/w) | | | | |

### Example 13

### Oleic Acid Based Microemulsion Formulations of Compound I

The following example demonstrates methods for preparing oleic acid-based microemulsion formulations that comprise Compound I.

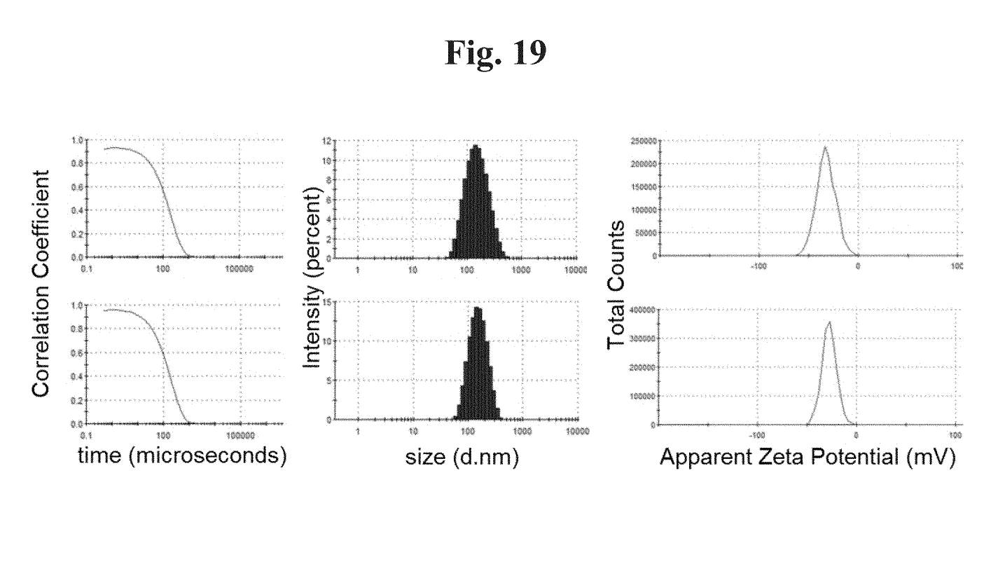

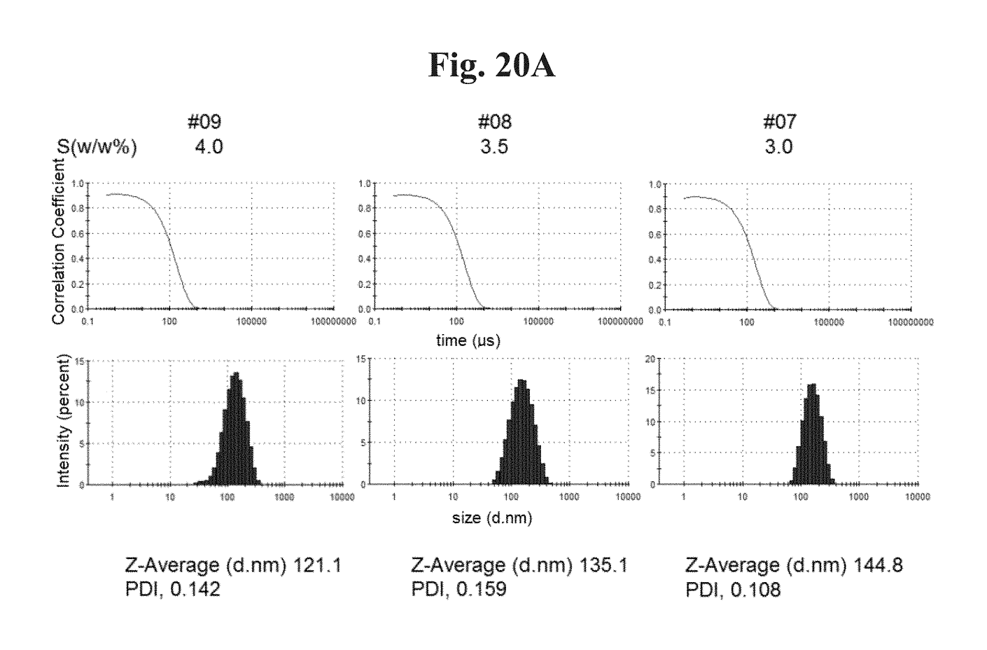

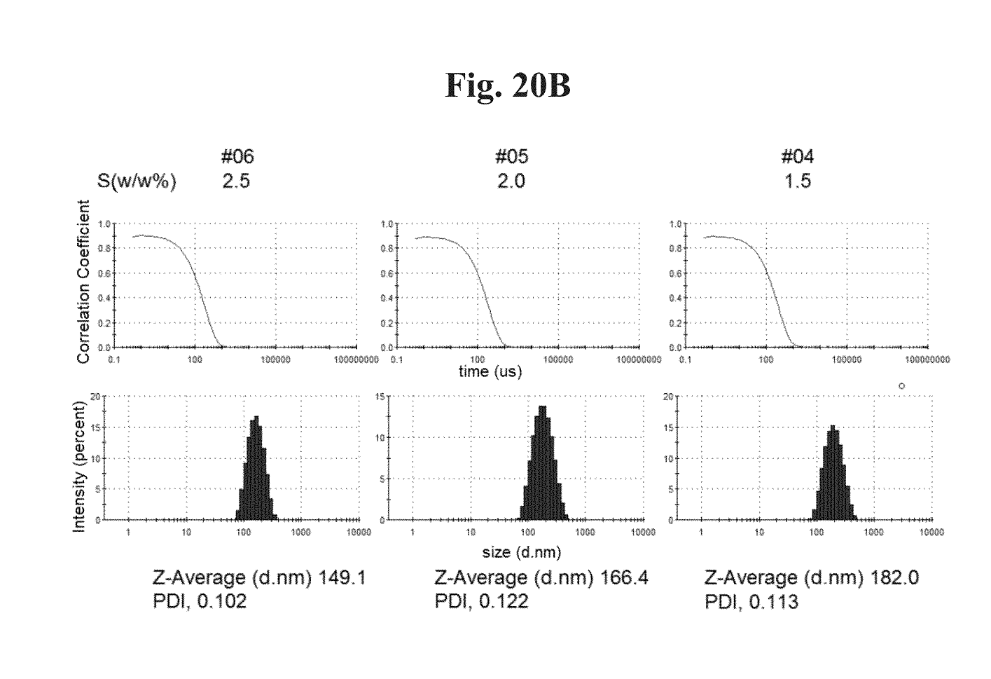

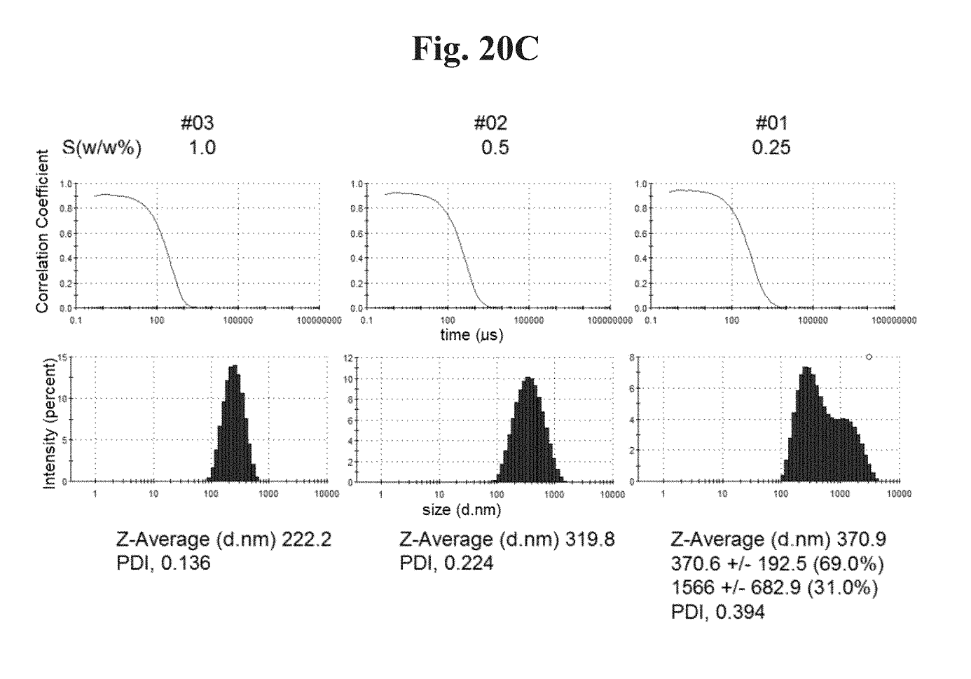

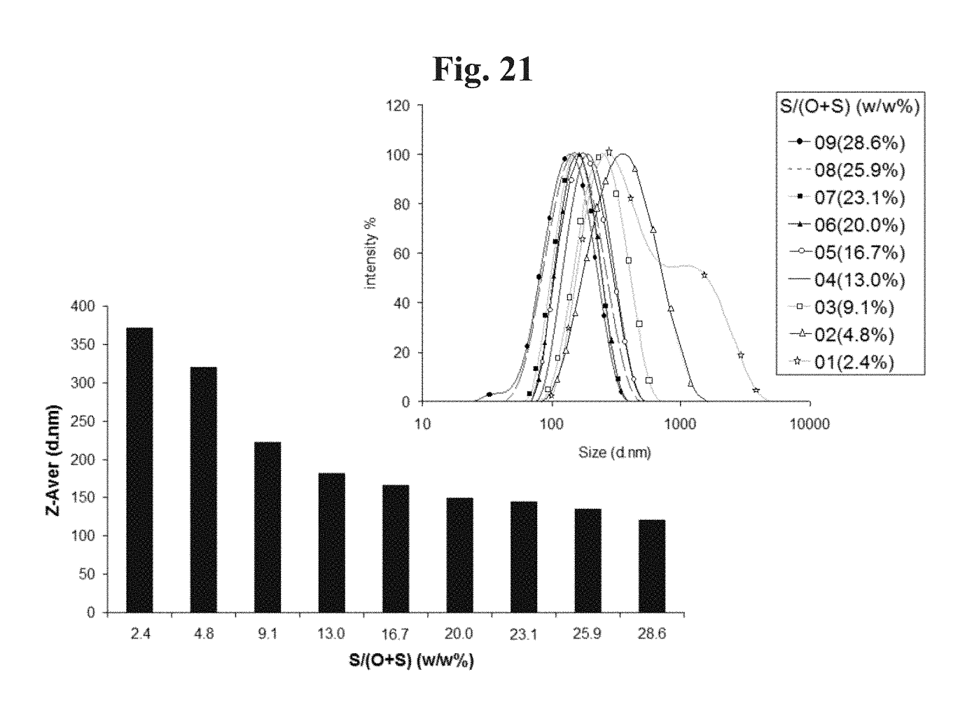

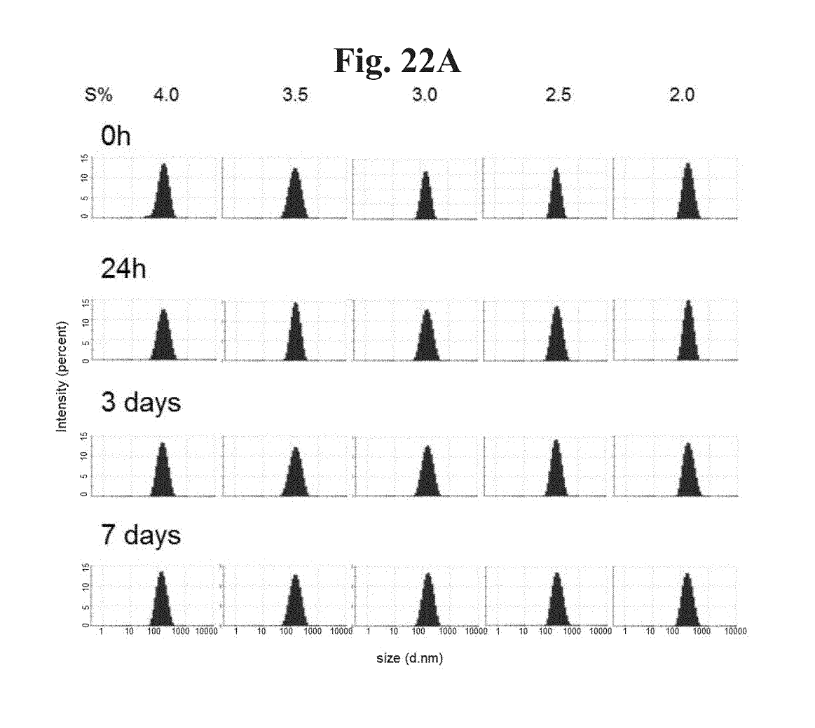

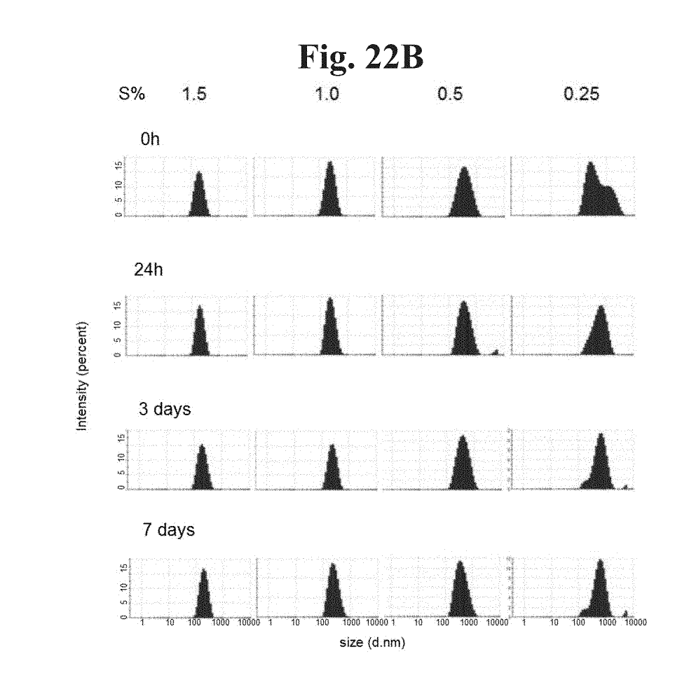

An emulsion of oleic acid (10% w/w) alone or saturated with Compound I (51.8 mg/ml) in various amounts of polysorbate 80, including from 0.25-4.0% (w/w) in deionized water was homogenized with a high speed homogenizer (Biospec Products, Inc.) at 30,000 rpm for 5 minutes. The homogenate was subjected to high-pressure homogenization using pneumatically controlled EmulsiFlex-C3 (Avestin) at 5000 psi in recirculating mode for 15 to 20 minutes. Microemulsions were characterized for particle size distribution and zeta-potential by ZetasizerNano-ZS (Malvern). Photon correlation microscopy (PCS) indicated identical size of microemulsions for empty microemulsion (133 nm (PDI 0.178)) and microemulsions comprising Compound I (133 nm (PDI 0.167)) (FIGS. 19, 20A-20C, and 21). Stability of Compound I-loaded microemulsions was assessed over time (FIGS. 22A and 22B). Microemulsions comprising Compound I showed a modest decrease of surface charge compared to control (−27.5 mV for microemulsions comprising Compound I compared to −32.5 mV for control microemulsions). Microemulsions with a Z-average of less than 150 nm and polysorbate 80 at 3.5% (w/w) showed stable particle size distribution profiles over several months for both control and Compound I microemulsions (FIG. 23).

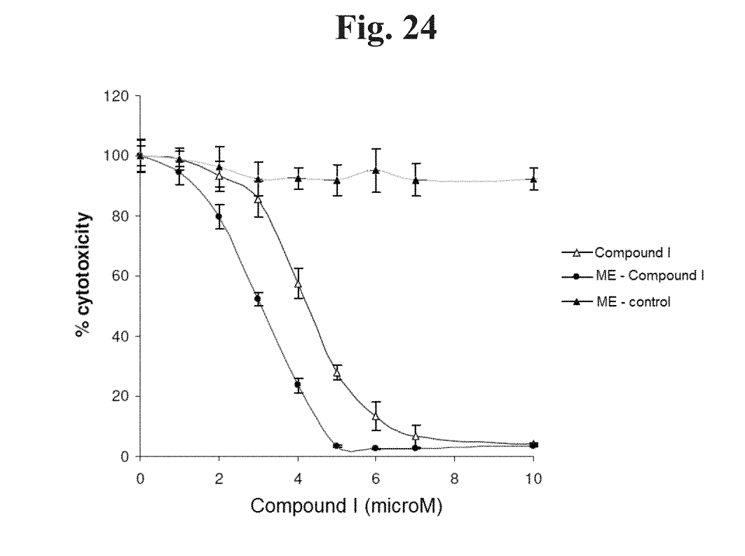

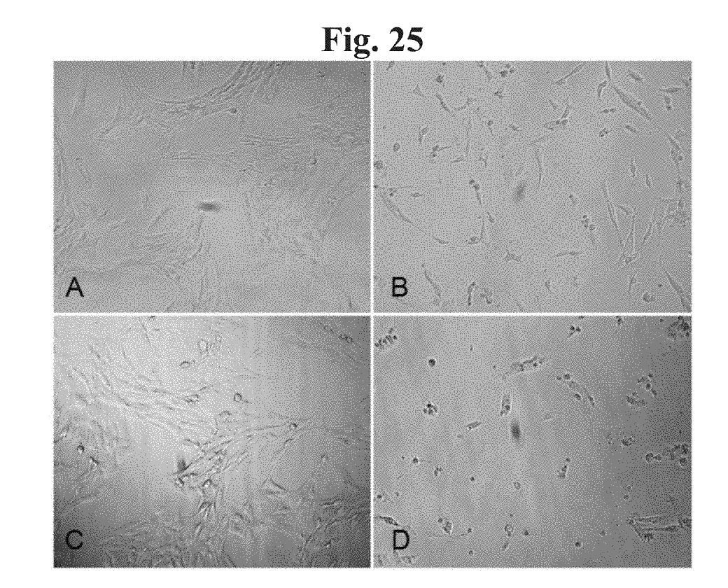

To analyze an antiproliferative activity of microemulsions comprising Compound I, prostate carcinoma cells P2-PTEN were exposed to increasing dilutions of: a) control microemulsions (ME—control); b) microemulsions comprising Compound I (ME—Compound I); and c) free Compound I. The cytotoxicity was determined after a 24 hour exposure using a formazan-based assay (CellTiter 96 AQueous™ on solution cell proliferation assay, Promega). Results showed that microemulsions comprising Compound I demonstrated significantly increased cytotoxicity compared to free Compound I (FIGS. 24 and 25). Control microemulsions without Compound I did not show any apparent cytotoxicity in the tested range.

It is to be understood that the description, specific examples and data, while indicating exemplary embodiments, are given by way of illustration and are not intended to limit the various embodiments of the present disclosure. Various changes and modifications within the present disclosure will become apparent to the skilled artisan from the description and data contained herein, and thus are considered part of the various embodiments of this disclosure.

1. A method for increasing survival of a subject or treating a subject suffering from prostate cancer, comprising:
identifying a subject at risk for reduced prostate cancer survival or a subject having prostate cancer;
administering to the subject a pharmaceutical composition comprising Compound I and at least one pharmaceutically acceptable carrier;
wherein Compound I is 5-hydroxy-2-methylnaphthalene-1,4-dione;
wherein the at least one pharmaceutically acceptable carrier comprises a mixture of triacylglycerols, wherein the triacylglycerols comprise glyceryl esters of one or more fatty acids selected from the group consisting of caproic acid, caprylic acid, capric acid, lauric acid, myristic acid, palmitic acid, stearic acid, oleic acid, linolenic acid, linoleic acid, arachidic acid, ricinoleic acid, dihydroxystearic acid, behenic acid, ligoceric acid, erucic acid, and gondoic acid; and
wherein the pharmaceutically acceptable carrier is a liquid at 25° C.;
wherein Compound I is dissolved in the pharmaceutically acceptable carrier at a concentration between 0.05 to 100 mg of Compound I per mL of pharmaceutically acceptable carrier; and
wherein the administration of said pharmaceutical composition results in survival of the subject for more than 1 month.

2. The method of claim 1, wherein when Compound I is dissolved in the pharmaceutically acceptable carrier at a temperature between 25° C. and 37° C., the resulting mixture is a liquid having a viscosity between 20-250 milliPascal seconds (mPa·s).

3. (canceled)

4. The method of claim 1, wherein the subject at risk for reduced prostate cancer survival has a ruptured or broken prostate capsule, or metastatic prostate cancer.

5. The method of claim 1, wherein the subject at risk for reduced prostate cancer survival has previously undergone surgical castration.

6. The method of claim 1, further comprising administering to the subject androgen deprivation therapy that reduces the amount of androgen in the subject, wherein the androgen deprivation therapy is selected from the group consisting of abiraterone, finasteride, dutasteride, degarelix, and leuprolide.

7. (canceled)

8. The method of claim 1, wherein:
the mixture of triacylglycerols comprise glyceryl esters of oleic acid, linoleic acid, palmitic acid, stearic acid, and arachidic acid; or
the mixture of triacylglycerols comprise glyceryl esters of ricinoleic acid, oleic acid, linoleic acid, palmitic acid, stearic acid, and dihydroxystearic acid; or
the mixture of triacylglycerols comprise glyceryl esters of lauric acid, myristic acid, palmitic acid, oleic acid, caprylic acid, stearic acid, capric acid, caproic acid, linoleic acid, and arachidic acid; or
the mixture of triacylglycerols comprise glyceryl esters of linoleic acid, oleic acid, palmitic acid, capric acid, caprylic acid, stearic acid, and myristic acid; or
the mixture of triacylglycerols comprise glyceryl esters of linoleic acid, oleic acid, palmitic acid, stearic acid, and myristic acid; or
the mixture of triacylglycerols comprise glyceryl esters of linoleic acid, oleic acid, palmitic acid, stearic acid, alpha-linolenic acid, and palmitoleic acid; or
the mixture of triacylglycerols comprise glyceryl esters of oleic acid, palmitic acid, linoleic acid, stearic acid, myristic acid, and arachidic acid; or
the mixture of triacylglycerols comprise glyceryl esters of oleic acid, linoleic acid, palmitic acid, stearic acid, behenic acid, arachidic acid, and lignoceric acid; or
the mixture of triacylglycerols comprise glyceryl esters of erucic acid, oleic acid, gondonic acid, linoleic acid, alpha-linolenic acid, palmitic acid, and stearic acid; or
the mixture of triacylglycerols comprise glyceryl esters of oleic acid, linoleic acid, alpha-linolenic acid, palmitic acid, gondonic acid, and stearic acid; or
the mixture of triacylglycerols comprise glyceryl esters of linoleic acid, oleic acid, palmitic acid, and stearic acid; or
the mixture of triacylglycerols comprise glyceryl esters of linoleic acid, oleic acid, palmitic acid, linolenic acid, and stearic acid; or
the mixture of triacylglycerols comprise glyceryl esters of linoleic acid, oleic acid, palmitic acid, stearic acid, arachidic acid, and behenic acid.

9. The method of claim 8, wherein:
the mixture of triacylglycerols comprise glyceryl esters having a fatty acid content of 40-50% oleic acid, 35-45% linoleic acid, 7-9% palmitic acid, 4-5% stearic acid, and 0.4-1% arachidic acid; or
the mixture of triacylglycerols comprise glyceryl esters having a fatty acid content of 85-95% ricinoleic acid, 2-8% oleic acid, 1-6% linoleic acid, 0.5-3% palmitic acid, 0.5-1% stearic acid, and 0.3-0.7% dihydroxystearic acid; or
the mixture of triacylglycerols comprise glyceryl esters having a fatty acid content of 44-52% lauric acid, 13-19% myristic acid, 8-11% palmitic acid, 6-10% capric acid, 5-8% oleic acid, 5-9% caprylic acid, 1-3% stearic acid, 0-1% linoleic acid, 0-0.8% caproic acid, and 0-0.5% arachidic acid; or
the mixture of triacylglycerols comprise glyceryl esters having a fatty acid content of 34-62% linoleic acid, 19-49% oleic acid, 8-12% palmitic acid, 7% capric acid, 4% caprylic acid, 2-5% stearic acid, and 0.2-1% myristic acid; or
the mixture of triacylglycerols comprise glyceryl esters having a fatty acid content of 40-63% linoleic acid, 13-44% oleic acid, 17-29% palmitic acid, 1-4% stearic acid, 0.5-2% myristic acid, and 0.1-2% alpha-linolenic acid; or
the mixture of triacylglycerols comprise glyceryl esters having a fatty acid content of 60-75% linoleic acid, 12-25% oleic acid, 6-9% palmitic acid, 3-6% stearic acid, 0-1.5% alpha-linolenic acid, and 0-1% palmitoleic acid; or
the mixture of triacylglycerols comprise glyceryl esters having a fatty acid content of 65-80% oleic acid, 7-16% palmitic acid, 4-10% linoleic acid, 1-3% stearic acid, 0.1-1% myristic acid, and 0.1-0.3% arachidic acid; or
the mixture of triacylglycerols comprise glyceryl esters having a fatty acid content of 38-52% oleic acid, 32-45% palmitic acid, 5-11% linoleic acid, 2-7% stearic acid, and 0.5-2% myristic acid; or
the mixture of triacylglycerols comprise glyceryl esters having a fatty acid content of 47-56% oleic acid, 26-33% linoleic acid, and 8-10% palmitic acid; or
the mixture of triacylglycerols comprise glyceryl esters having a fatty acid content of 41% erucic acid, 17% oleic acid, 15% gondonic acid, 13% linoleic acid, 9% alpha-linolenic acid, 4% palmitic acid, and 1.5% stearic acid; or
the mixture of triacylglycerols comprise glyceryl esters having a fatty acid content of 61-63% oleic acid, 20-21% linoleic acid, 9-11% alpha-linolenic acid, 4% palmitic acid, 2% gondonic acid, 2% stearic acid, and less than 2% erucic acid; or
the mixture of triacylglycerols comprise glyceryl esters having a fatty acid content of 73-79% linoleic acid, 13-21% oleic acid, 3-6% palmitic acid, and 1-4% stearic acid; or
the mixture of triacylglycerols comprise glyceryl esters having a fatty acid content of 43-56% linoleic acid, 22-34% oleic acid, 7-11% palmitic acid, 5-11% linolenic acid, and 2-6% stearic acid; or
the mixture of triacylglycerols comprise glyceryl esters having a fatty acid content of 44-75% linoleic acid, 14-35% oleic acid, 3-6% palmitic acid, 1-3% stearic acid, 0.6-4% arachidic acid, and 1% behenic acid.

10-12. (canceled)

13. The method of claim 1, wherein the subject is administered a dosage amount of 1 mg of Compound I per kg of subject, and the pharmaceutical composition is formulated for oral administration to the patient.

14. A method for inhibiting, delaying, or preventing rupture of a subject's prostatic capsule in a subject suffering from prostate cancer, comprising:
administering to the subject a pharmaceutical composition comprising Compound I and at least one pharmaceutically acceptable carrier;
wherein Compound I is 5-hydroxy-2-methylnaphthalene-1,4-dione;
wherein the at least one pharmaceutically acceptable carrier comprises a mixture of triacylglycerols, wherein the triacylglycerols comprise glyceryl esters of one or more fatty acids selected from the group consisting of caproic acid, caprylic acid, capric acid, lauric acid, myristic acid, palmitic acid, stearic acid, oleic acid, linolenic acid, linoleic acid, arachidic acid, ricinoleic acid, dihydroxystearic acid, behenic acid, ligoceric acid, erucic acid, and gondoic acid; and
wherein the pharmaceutically acceptable carrier is a liquid at 25° C.;
wherein Compound I is dissolved in the pharmaceutically acceptable carrier at a concentration between 0.05 to 100 mg of Compound I per mL of pharmaceutically acceptable carrier.

15. The method of claim 14, wherein when Compound I is dissolved in the pharmaceutically acceptable carrier at a temperature between 25° C. and 37° C., the resulting mixture is a liquid having a viscosity between 20-250 milliPascal seconds (mPa·s).

16. (canceled)

17. The method of claim 14, wherein the subject at risk for reduced prostate cancer survival has previously undergone surgical castration.

18. The method of claim 14, further comprising administering to the subject androgen deprivation therapy that reduces the amount of androgen in the subject, wherein the androgen deprivation therapy is selected from the group consisting of abiraterone, finasteride, dutasteride, degarelix, and leuprolide.

19. (canceled)

20. The method of claim 14, wherein:
the mixture of triacylglycerols comprise glyceryl esters of oleic acid, linoleic acid, palmitic acid, stearic acid, and arachidic acid; or
the mixture of triacylglycerols comprise glyceryl esters of ricinoleic acid, oleic acid, linoleic acid, palmitic acid, stearic acid, and dihydroxystearic acid; or
the mixture of triacylglycerols comprise glyceryl esters of lauric acid, myristic acid, palmitic acid, oleic acid, caprylic acid, stearic acid, capric acid, caproic acid, linoleic acid, and arachidic acid; or
the mixture of triacylglycerols comprise glyceryl esters of linoleic acid, oleic acid, palmitic acid, capric acid, caprylic acid, stearic acid, and myristic acid; or
the mixture of triacylglycerols comprise glyceryl esters of linoleic acid, oleic acid, palmitic acid, stearic acid, and myristic acid; or
the mixture of triacylglycerols comprise glyceryl esters of linoleic acid, oleic acid, palmitic acid, stearic acid, alpha-linolenic acid, and palmitoleic acid; or
the mixture of triacylglycerols comprise glyceryl esters of oleic acid, palmitic acid, linoleic acid, stearic acid, myristic acid, and arachidic acid; or
the mixture of triacylglycerols comprise glyceryl esters of oleic acid, linoleic acid, palmitic acid, stearic acid, behenic acid, arachidic acid, and lignoceric acid; or
the mixture of triacylglycerols comprise glyceryl esters of erucic acid, oleic acid, gondonic acid, linoleic acid, alpha-linolenic acid, palmitic acid, and stearic acid; or
the mixture of triacylglycerols comprise glyceryl esters of oleic acid, linoleic acid, alpha-linolenic acid, palmitic acid, gondonic acid, and stearic acid; or
the mixture of triacylglycerols comprise glyceryl esters of linoleic acid, oleic acid, palmitic acid, and stearic acid; or
the mixture of triacylglycerols comprise glyceryl esters of linoleic acid, oleic acid, palmitic acid, linolenic acid, and stearic acid; or
the mixture of triacylglycerols comprise glyceryl esters of linoleic acid, oleic acid, palmitic acid, stearic acid, arachidic acid, and behenic acid.

21. The method of claim 20, wherein:
the mixture of triacylglycerols comprise glyceryl esters having a fatty acid content of 40-50% oleic acid, 35-45% linoleic acid, 7-9% palmitic acid, 4-5% stearic acid, and 0.4-1% arachidic acid; or
the mixture of triacylglycerols comprise glyceryl esters having a fatty acid content of 85-95% ricinoleic acid, 2-8% oleic acid, 1-6% linoleic acid, 0.5-3% palmitic acid, 0.5-1% stearic acid, and 0.3-0.7% dihydroxystearic acid; or
the mixture of triacylglycerols comprise glyceryl esters having a fatty acid content of 44-52% lauric acid, 13-19% myristic acid, 8-11% palmitic acid, 6-10% capric acid, 5-8% oleic acid, 5-9% caprylic acid, 1-3% stearic acid, 0-1% linoleic acid, 0-0.8% caproic acid, and 0-0.5% arachidic acid; or
the mixture of triacylglycerols comprise glyceryl esters having a fatty acid content of 34-62% linoleic acid, 19-49% oleic acid, 8-12% palmitic acid, 7% capric acid, 4% caprylic acid, 2-5% stearic acid, and 0.2-1% myristic acid; or
the mixture of triacylglycerols comprise glyceryl esters having a fatty acid content of 40-63% linoleic acid, 13-44% oleic acid, 17-29% palmitic acid, 1-4% stearic acid, 0.5-2% myristic acid, and 0.1-2% alpha-linolenic acid; or
the mixture of triacylglycerols comprise glyceryl esters having a fatty acid content of 60-75% linoleic acid, 12-25% oleic acid, 6-9% palmitic acid, 3-6% stearic acid, 0-1.5% alpha-linolenic acid, and 0-1% palmitoleic acid; or
the mixture of triacylglycerols comprise glyceryl esters having a fatty acid content of 65-80% oleic acid, 7-16% palmitic acid, 4-10% linoleic acid, 1-3% stearic acid, 0.1-1% myristic acid, and 0.1-0.3% arachidic acid; or
the mixture of triacylglycerols comprise glyceryl esters having a fatty acid content of 38-52% oleic acid, 32-45% palmitic acid, 5-11% linoleic acid, 2-7% stearic acid, and 0.5-2% myristic acid; or
the mixture of triacylglycerols comprise glyceryl esters having a fatty acid content of 47-56% oleic acid, 26-33% linoleic acid, and 8-10% palmitic acid; or
the mixture of triacylglycerols comprise glyceryl esters having a fatty acid content of 41% erucic acid, 17% oleic acid, 15% gondonic acid, 13% linoleic acid, 9% alpha-linolenic acid, 4% palmitic acid, and 1.5% stearic acid; or
the mixture of triacylglycerols comprise glyceryl esters having a fatty acid content of 61-63% oleic acid, 20-21% linoleic acid, 9-11% alpha-linolenic acid, 4% palmitic acid, 2% gondonic acid, 2% stearic acid, and less than 2% erucic acid; or
the mixture of triacylglycerols comprise glyceryl esters having a fatty acid content of 73-79% linoleic acid, 13-21% oleic acid, 3-6% palmitic acid, and 1-4% stearic acid; or
the mixture of triacylglycerols comprise glyceryl esters having a fatty acid content of 43-56% linoleic acid, 22-34% oleic acid, 7-11% palmitic acid, 5-11% linolenic acid, and 2-6% stearic acid; or
the mixture of triacylglycerols comprise glyceryl esters having a fatty acid content of 44-75% linoleic acid, 14-35% oleic acid, 3-6% palmitic acid, 1-3% stearic acid, 0.6-4% arachidic acid, and 1% behenic acid.

22-24. (canceled)

25. The method of claim 14, wherein the subject is administered a dosage amount of 1 mg of Compound I per kg of subject, and the pharmaceutical composition is formulated for oral administration to the patient.

26. A pharmaceutical composition comprising:
Compound I and at least one pharmaceutically acceptable carrier;
wherein Compound I is 5-hydroxy-2-methylnaphthalene-1,4-dione;
wherein the at least one pharmaceutically acceptable carrier comprises a mixture of triacylglycerols, wherein the triacylglycerols comprise glyceryl esters of one or more fatty acids selected from the group consisting of caproic acid, caprylic acid, capric acid, lauric acid, myristic acid, palmitic acid, stearic acid, oleic acid, linolenic acid, linoleic acid, arachidic acid, ricinoleic acid, dihydroxystearic acid, behenic acid, ligoceric acid, erucic acid, and gondoic acid; and
wherein the pharmaceutically acceptable carrier is a liquid at 25° C.;
wherein Compound I is dissolved in the pharmaceutically acceptable carrier at a concentration between 0.05 to 100 mg of Compound I per mL of pharmaceutically acceptable carrier.

27. The pharmaceutical composition of claim 26, wherein when Compound I is dissolved in the pharmaceutically acceptable carrier at a temperature between 25° C. and 37° C., the resulting mixture is a liquid having a viscosity between 20-250 milliPascal seconds (mPa·s).

28. (canceled)

29. The pharmaceutical composition of claim 26, wherein:
the mixture of triacylglycerols comprise glyceryl esters of oleic acid, linoleic acid, palmitic acid, stearic acid, and arachidic acid; or
the mixture of triacylglycerols comprise glyceryl esters of ricinoleic acid, oleic acid, linoleic acid, palmitic acid, stearic acid, and dihydroxystearic acid; or
the mixture of triacylglycerols comprise glyceryl esters of lauric acid, myristic acid, palmitic acid, oleic acid, caprylic acid, stearic acid, capric acid, caproic acid, linoleic acid, and arachidic acid; or
the mixture of triacylglycerols comprise glyceryl esters of linoleic acid, oleic acid, palmitic acid, capric acid, caprylic acid, stearic acid, and myristic acid; or
the mixture of triacylglycerols comprise glyceryl esters of linoleic acid, oleic acid, palmitic acid, stearic acid, and myristic acid; or
the mixture of triacylglycerols comprise glyceryl esters of linoleic acid, oleic acid, palmitic acid, stearic acid, alpha-linolenic acid, and palmitoleic acid; or
the mixture of triacylglycerols comprise glyceryl esters of oleic acid, palmitic acid, linoleic acid, stearic acid, myristic acid, and arachidic acid; or
the mixture of triacylglycerols comprise glyceryl esters of oleic acid, linoleic acid, palmitic acid, stearic acid, behenic acid, arachidic acid, and lignoceric acid; or
the mixture of triacylglycerols comprise glyceryl esters of erucic acid, oleic acid, gondonic acid, linoleic acid, alpha-linolenic acid, palmitic acid, and stearic acid; or
the mixture of triacylglycerols comprise glyceryl esters of oleic acid, linoleic acid, alpha-linolenic acid, palmitic acid, gondonic acid, and stearic acid; or
the mixture of triacylglycerols comprise glyceryl esters of linoleic acid, oleic acid, palmitic acid, and stearic acid; or
the mixture of triacylglycerols comprise glyceryl esters of linoleic acid, oleic acid, palmitic acid, linolenic acid, and stearic acid; or
the mixture of triacylglycerols comprise glyceryl esters of linoleic acid, oleic acid, palmitic acid, stearic acid, arachidic acid, and behenic acid.

30. The pharmaceutical composition of claim 29, wherein:
the mixture of triacylglycerols comprise glyceryl esters having a fatty acid content of 40-50% oleic acid, 35-45% linoleic acid, 7-9% palmitic acid, 4-5% stearic acid, and 0.4-1% arachidic acid; or
the mixture of triacylglycerols comprise glyceryl esters having a fatty acid content of 85-95% ricinoleic acid, 2-8% oleic acid, 1-6% linoleic acid, 0.5-3% palmitic acid, 0.5-1% stearic acid, and 0.3-0.7% dihydroxystearic acid; or
the mixture of triacylglycerols comprise glyceryl esters having a fatty acid content of 44-52% lauric acid, 13-19% myristic acid, 8-11% palmitic acid, 6-10% capric acid, 5-8% oleic acid, 5-9% caprylic acid, 1-3% stearic acid, 0-1% linoleic acid, 0-0.8% caproic acid, and 0-0.5% arachidic acid; or
the mixture of triacylglycerols comprise glyceryl esters having a fatty acid content of 34-62% linoleic acid, 19-49% oleic acid, 8-12% palmitic acid, 7% capric acid, 4% caprylic acid, 2-5% stearic acid, and 0.2-1% myristic acid; or
the mixture of triacylglycerols comprise glyceryl esters having a fatty acid content of 40-63% linoleic acid, 13-44% oleic acid, 17-29% palmitic acid, 1-4% stearic acid, 0.5-2% myristic acid, and 0.1-2% alpha-linolenic acid; or
the mixture of triacylglycerols comprise glyceryl esters having a fatty acid content of 60-75% linoleic acid, 12-25% oleic acid, 6-9% palmitic acid, 3-6% stearic acid, 0-1.5% alpha-linolenic acid, and 0-1% palmitoleic acid; or
the mixture of triacylglycerols comprise glyceryl esters having a fatty acid content of 65-80% oleic acid, 7-16% palmitic acid, 4-10% linoleic acid, 1-3% stearic acid, 0.1-1% myristic acid, and 0.1-0.3% arachidic acid; or
the mixture of triacylglycerols comprise glyceryl esters having a fatty acid content of 38-52% oleic acid, 32-45% palmitic acid, 5-11% linoleic acid, 2-7% stearic acid, and 0.5-2% myristic acid; or
the mixture of triacylglycerols comprise glyceryl esters having a fatty acid content of 47-56% oleic acid, 26-33% linoleic acid, and 8-10% palmitic acid; or
the mixture of triacylglycerols comprise glyceryl esters having a fatty acid content of 41% erucic acid, 17% oleic acid, 15% gondonic acid, 13% linoleic acid, 9% alpha-linolenic acid, 4% palmitic acid, and 1.5% stearic acid; or
the mixture of triacylglycerols comprise glyceryl esters having a fatty acid content of 61-63% oleic acid, 20-21% linoleic acid, 9-11% alpha-linolenic acid, 4% palmitic acid, 2% gondonic acid, 2% stearic acid, and less than 2% erucic acid; or
the mixture of triacylglycerols comprise glyceryl esters having a fatty acid content of 73-79% linoleic acid, 13-21% oleic acid, 3-6% palmitic acid, and 1-4% stearic acid; or
the mixture of triacylglycerols comprise glyceryl esters having a fatty acid content of 43-56% linoleic acid, 22-34% oleic acid, 7-11% palmitic acid, 5-11% linolenic acid, and 2-6% stearic acid; or
the mixture of triacylglycerols comprise glyceryl esters having a fatty acid content of 44-75% linoleic acid, 14-35% oleic acid, 3-6% palmitic acid, 1-3% stearic acid, 0.6-4% arachidic acid, and 1% behenic acid.

31-32. (canceled)

33. The pharmaceutical composition of claim 26, wherein the amount of Compound I in the pharmaceutical composition is selected from the group consisting of 25 mg, 50 mg, 75 mg, 100 mg, 125 mg, 150 mg, 200 mg, and 250 mg.

34-57. (canceled)
